(19) United States
(12) Patent Application Publication
(10) Pub. No.: US 2026/0201344 A1
(43) Pub. Date: Jul. 16, 2026

(54) STABLE PRODUCTION SYSTEMS FOR ADENO-ASSOCIATED VIRUS PRODUCTION

(71) Applicant: Asimov Inc., Boston, MA (US)

(72) Inventors: Michael T. Leonard, Boston, MA (US); Jeremy J. Gam, Somerville, MA (US); Alec A.K. Nielsen, Brookline, MA (US)

(73) Assignee: Asimov Inc., Boston, MA (US)

(21) Appl. No.: 19/128,186

(22) PCT Filed: Nov. 10, 2023
(86) PCT No.: PCT/US2023/079341

§ 371 Date: May 8, 2025

Related U.S. Application Data
(60) Provisional application No. 63/424,450, filed on Nov. 10, 2022.

(51) Int. Cl. C12N 7/02; C07K 14/015; C12N 5/071; C12N 9/22; C12N 15/113; C12N 15/864

(52) U.S. Cl. CPC C12N 7/025; C07K 14/015; C12N 5/0686; C12N 9/226; C12N 15/1131; C12N 15/8645; C12N 2310/20; C12N 2510/04; C12N 2750/14143; C12N 2750/14152; C12N 2800/10; C12N 2830/34; C12N 2830/36

(57) ABSTRACT

Disclosed herein are genetically engineered cells for AAV production. The genetically engineered cell comprises molecular systems for temporal control of expression of genes required for AAV production. Also disclosed herein are methods of using genetically engineered cells for AAV production.

### RELATED APPLICATIONS

This application claims the benefit under 35 U.S.C. § 119 of U.S. provisional application Ser. No. 63/424,450, filed Nov. 10, 2022, the entire contents of which are incorporated by reference herein.

### FIELD

Described herein are Adeno-Associated Virus (AAV) production systems. Also described herein are engineered cells and kits comprising an AAV production system and methods of using the same for AAV production.

### REFERENCE TO AN ELECTRONIC SEQUENCE LISTING

The contents of the electronic sequence listing (A121070012WO00-SEQ-CRP.xml; Size: 72,389 bytes; and Date of Creation: Nov. 9, 2023) is herein incorporated by reference in its entirety.

### BACKGROUND

AAV are a promising gene delivery modality for cell and gene therapy. AAV can be modified to carry therapeutic genetic payloads to cells within a subject. The production of AAV normally entails transient transfection of plasmids containing genes required for viral vector production into cell culture. However, transient transfection has several shortfalls. Large quantities of DNA and transfection reagent must be procured for the transfection process, which is costly. Also, poor transfection efficiency can result in minimal numbers of ‘transfected’ cells and increased variation associated with transfection steps and viral production.

### SUMMARY

Described herein are AAV production systems that introduce inducible control of gene products required for AAV production including cytostatic or cytotoxic gene products. This inducible control can be mediated at the genomic level (i.e., inducible control of genomic modification) or at the translational level (i.e., inducible control of altered translation). Each of the described AAV production systems can be integrated into the genome using random integration, targeted integration, or transposon-mediated integration.

In some aspects, the disclosure relates to an engineered cell for AAV production, comprising one or more stably integrated nucleic acid molecules collectively comprising a nucleic acid sequence encoding for each of: Rep52, DA-Rep52, Rep40, or DA-Rep40; Rep78, DA-Rep78, Rep68, or DA-Rep68; E2A or DA-E2A; E4ORF6 or DA-E4ORF6; VARNA or DA-VARNA; VP1 or DA-VP1; VP2 or DA-VP2; VP3 or DA-VP3; AAP or DA-AAP; MAAP or DA-MAAP; and L4 100K or DA-L4 100K and a Base Editor, each nucleic acid molecule being operably linked to a promoter; wherein the cell comprises the nucleic acid sequence of at least one of DA-Rep52, DA-Rep40, DA-Rep78, DA-Rep68, DA-E2A, DA-E4ORF6, DA-VP1, DA-VP2, DA-VP3, DA-AAP, DA-MAAP, and DA-L4 100K; wherein the nucleic acid sequences of DA-Rep52, DA-Rep40, DA-Rep78, DA-Rep68, DA-E2A, DA-E4ORF6, DA-VP1, DA-VP2, DA-VP3, DA-AAP, DA-MAAP, and DA-L4 100K each comprises a mutated start codon, a non-synonymous mutation, or a premature stop codon.

In some embodiments, the AAV production system comprises a nucleic acid sequence encoding for DA-Rep52, wherein the DA-Rep52 has at least 80% identity with SEQ ID NO: 6, and wherein the amino acid at position 1 of DA-Rep52 is: (i) an isoleucine residue encoded by an ATA codon; (ii) a threonine residue encoded by an ACG codon; (iii) a valine residue encoded by a GTG codon; or (iv) an alanine codon encoded by a GCG codon.

In some embodiments, the nucleic acid sequence encoding for DA-Rep52 further comprises one or more non-synonymous codons.

In some embodiments, the AAV production system further comprises a nucleic acid sequence encoding for DA-Rep52, wherein the DA-Rep52 has at least 80% identity with SEQ ID NO: 6, and wherein: (i) a tryptophan residue of DA-Rep52 is replaced with a TGA stop codon, a TAG stop codon, or a TAA stop codon; (ii) a glutamine residue of DA-Rep52 is replaced with a TAG stop codon or a TAA stop codon; or (iii) an arginine residue of DA-Rep52 is replaced with a TGA stop codon.

In some embodiments, the AAV production system comprises a nucleic acid sequence encoding for DA-Rep40, wherein the DA-Rep40 has at least 80% identity with SEQ ID NO: 7, and wherein the amino acid at position 1 of DA-Rep40 is: (i) an isoleucine residue encoded by an ATA codon; (ii) a threonine residue encoded by an ACG codon; (iii) a valine residue encoded by a GTG codon; or (iv) an alanine codon encoded by a GCG codon.

In some embodiments, the nucleic acid sequence encoding for DA-Rep40 further comprises one or more non-synonymous codons.

In some embodiments, the AAV production system further comprises a nucleic acid sequence encoding for DA-Rep40, wherein the DA-Rep40 has at least 80% identity with SEQ ID NO: 7, and wherein: (i) a tryptophan residue of DA-Rep40 is replaced with a TGA stop codon, a TAG stop codon, or a TAA stop codon; (ii) a glutamine residue of DA-Rep40 is replaced with a TAG stop codon or a TAA stop codon; or (iii) an arginine residue of DA-Rep40 is replaced with a TGA stop codon.

In some embodiments, the AAV production system comprises a nucleic acid sequence encoding for DA-Rep78, wherein the DA-Rep78 has at least 80% identity with SEQ ID NO: 8, and wherein the amino acid at position 1 of DA-Rep78 is: (i) an isoleucine residue encoded by an ATA codon; (ii) a threonine residue encoded by an ACG codon; (iii) a valine residue encoded by a GTG codon; or (iv) an alanine codon encoded by a GCG codon.

In some embodiments, the nucleic acid sequence encoding for DA-Rep78 further comprises one or more non-synonymous codons.

In some embodiments, the AAV production system further comprises a nucleic acid sequence encoding for DA-Rep78, wherein the DA-Rep78 has at least 80% identity with SEQ ID NO: 8, and wherein: (i) a tryptophan residue of DA-Rep78 is replaced with a TGA stop codon, a TAG stop codon, or a TAA stop codon; (ii) a glutamine residue of DA-Rep78 is replaced with a TAG stop codon or a TAA stop codon; or (iii) an arginine residue of DA-Rep78 is replaced with a TGA stop codon.

In some embodiments, the AAV production system comprises a nucleic acid sequence encoding for DA-Rep68, wherein the DA-Rep68 has at least 80% identity with SEQ ID NO: 9, and wherein the amino acid at position 1 of DA-Rep68 is: (i) an isoleucine residue encoded by an ATA codon; (ii) a threonine residue encoded by an ACG codon; (iii) a valine residue encoded by a GTG codon; or (iv) an alanine codon encoded by a GCG codon.

In some embodiments, the nucleic acid sequence encoding for DA-Rep68 further comprises one or more non-synonymous codons.

In some embodiments, the AAV production system further comprises a nucleic acid sequence encoding for DA-Rep68, wherein the DA-Rep68 has at least 80% identity with SEQ ID NO: 9, and wherein: (i) a tryptophan residue of DA-Rep68 is replaced with a TGA stop codon, a TAG stop codon, or a TAA stop codon; (ii) a glutamine residue of DA-Rep68 is replaced with a TAG stop codon or a TAA stop codon; or (iii) an arginine residue of DA-Rep68 is replaced with a TGA stop codon.

In some embodiments, the AAV production system comprises a nucleic acid sequence encoding for DA-E2A, wherein the DA-E2A has at least 80% identity with SEQ ID NO: 10, and wherein the amino acid at position 1 of DA-E2A is: (i) an isoleucine residue encoded by an ATA codon; (ii) a threonine residue encoded by an ACG codon; (iii) a valine residue encoded by a GTG codon; or (iv) an alanine codon encoded by a GCG codon.

In some embodiments, the nucleic acid sequence encoding for DA-E2A further comprises one or more non-synonymous codons.

In some embodiments, the AAV production system further comprises a nucleic acid sequence encoding for DA-E2A, wherein the DA-E2A has at least 80% identity with SEQ ID NO: 10, and wherein: (i) a tryptophan residue of DA-E2A is replaced with a TGA stop codon, a TAG stop codon, or a TAA stop codon; (ii) a glutamine residue of DA-E2A is replaced with a TAG stop codon or a TAA stop codon; or (iii) an arginine residue of DA-E2A is replaced with a TGA stop codon.

In some embodiments, the AAV production system comprises a nucleic acid sequence encoding for DA-E4ORF6, wherein the DA-E4ORF6 has at least 80% identity with SEQ ID NO: 11 or SEQ ID NO: 12, and wherein the amino acid at position 1 of DA-E4ORF6 is: (i) an isoleucine residue encoded by an ATA codon; (ii) a threonine residue encoded by an ACG codon; (iii) a valine residue encoded by a GTG codon; or (iv) an alanine codon encoded by a GCG codon.

In some embodiments, the nucleic acid sequence encoding for DA-E4ORF6 further comprises one or more non-synonymous codons.

In some embodiments, the AAV production system further comprises a nucleic acid sequence encoding for DA-E4ORF6 wherein the DA-E4ORF6 has at least 80% identity with SEQ ID NO: 11 or SEQ ID NO: 12, and wherein: (i) a tryptophan residue of DA-E4ORF6 is replaced with a TGA stop codon, a TAG stop codon, or a TAA stop codon; (ii) a glutamine residue of DA-E4ORF6 is replaced with a TAG stop codon or a TAA stop codon; or (iii) an arginine residue of DA-E4ORF6 is replaced with a TGA stop codon.

In some embodiments, the AAV production system comprises a nucleic acid sequence encoding for DA-VP1, wherein the DA-VP1 has at least 80% identity with SEQ ID NO: 14, and wherein the amino acid at position 1 of DA-VP1 is: (i) an isoleucine residue encoded by an ATA codon; (ii) a threonine residue encoded by an ACG codon; (iii) a valine residue encoded by a GTG codon; or (iv) an alanine codon encoded by a GCG codon.

In some embodiments, the nucleic acid sequence encoding for DA-VP1 further comprises one or more non-synonymous codons.

In some embodiments, the AAV production system further comprises a nucleic acid sequence encoding for DA-VP1 wherein the DA-VP1 has at least 80% identity with SEQ ID NO: 14, and wherein: (i) a tryptophan residue of DA-VP1 is replaced with a TGA stop codon, a TAG stop codon, or a TAA stop codon; (ii) a glutamine residue of DA-VP1 is replaced with a TAG stop codon or a TAA stop codon; or (iii) an arginine residue of DA-VP1 is replaced with a TGA stop codon.

In some embodiments, the AAV production system comprises a nucleic acid sequence encoding for DA-VP2, wherein the DA-VP2 has at least 80% identity with SEQ ID NO: 15, and wherein the amino acid at position 1 of DA-VP2 is: (i) a threonine residue encoded by an ACA codon; or (ii) an alanine codon encoded by a GCG codon.

In some embodiments, the nucleic acid sequence encoding for DA-VP2 further comprises one or more non-synonymous codons.

In some embodiments, the AAV production system further comprises a nucleic acid sequence encoding for DA-VP2 wherein the DA-VP2 has at least 80% identity with SEQ ID NO: 15, and wherein: (i) a tryptophan residue of DA-VP2 is replaced with a TGA stop codon, a TAG stop codon, or a TAA stop codon; (ii) a glutamine residue of DA-VP2 is replaced with a TAG stop codon or a TAA stop codon; or (iii) an arginine residue of DA-VP2 is replaced with a TGA stop codon.

In some embodiments, the AAV production system comprises a nucleic acid sequence encoding for DA-VP3, wherein the DA-VP3 has at least 80% identity with SEQ ID NO: 16, and wherein the amino acid at position 1 of DA-VP3 is: (i) an isoleucine residue encoded by an ATA codon; (ii) a threonine residue encoded by an ACG codon; (iii) a valine residue encoded by a GTG codon; or (iv) an alanine codon encoded by a GCG codon.

In some embodiments, the nucleic acid sequence encoding for DA-VP3 further comprises one or more non-synonymous codons.

In some embodiments, the AAV production system further comprises a nucleic acid sequence encoding for DA-VP3 wherein the DA-VP3 has at least 80% identity with SEQ ID NO: 16, and wherein: (i) a tryptophan residue of DA-VP3 is replaced with a TGA stop codon, a TAG stop codon, or a TAA stop codon; (ii) a glutamine residue of DA-VP3 is replaced with a TAG stop codon or a TAA stop codon; or (iii) an arginine residue of DA-VP3 is replaced with a TGA stop codon.

In some embodiments, the AAV production system comprises a nucleic acid sequence encoding for DA-AAP, wherein the DA-AAP has at least 80% identity with SEQ ID NO: 17, and wherein the amino acid at position 1 of DA-AAP is: (i) a leucine residue encoded by an CTA codon; (ii) a leucine residue encoded by an TTG codon; or (iii) a proline residue encoded by a CCG codon.

In some embodiments, the nucleic acid sequence encoding for DA-AAP further comprises one or more non-synonymous codons.

In some embodiments, the AAV production system further comprises a nucleic acid sequence encoding for DA-AAP wherein the DA-AAP has at least 80% identity with SEQ ID NO: 17, and wherein: (i) a tryptophan residue of DA-AAP is replaced with a TGA stop codon, a TAG stop codon, or a TAA stop codon; (ii) a glutamine residue of DA-AAP is replaced with a TAG stop codon or a TAA stop codon; or (iii) an arginine residue of DA-AAP is replaced with a TGA stop codon.

In some embodiments, the AAV production system comprises a nucleic acid sequence encoding for DA-MAAP, wherein the DA-MAAP has at least 80% identity with SEQ ID NO: 43, and wherein the amino acid at position 1 of DA-MAAP is: (i) a leucine residue encoded by an CTA codon; (ii) a leucine residue encoded by an TTG codon; or (iii) a proline residue encoded by a CCG codon.

In some embodiments, the nucleic acid sequence encoding for DA-MAAP further comprises one or more non-synonymous codons.

In some embodiments, the AAV production system further comprises a nucleic acid sequence encoding for DA-MAAP wherein the DA-MAAP has at least 80% identity with SEQ ID NO: 43, and wherein: (i) a tryptophan residue of DA-MAAP is replaced with a TGA stop codon, a TAG stop codon, or a TAA stop codon; (ii) a glutamine residue of DA-MAAP is replaced with a TAG stop codon or a TAA stop codon; or (iii) an arginine residue of DA-MAAP is replaced with a TGA stop codon.

In some embodiments, the AAV production system comprises a nucleic acid sequence encoding for DA-L4 100K, wherein the DA-L4 100K has at least 80% identity with SEQ ID NO: 39, and wherein the amino acid at position 1 of DA-L4 100K is: (i) an isoleucine residue encoded by an ATA codon; (ii) a threonine residue encoded by an ACG codon; (iii) a valine residue encoded by a GTG codon; or (iv) an alanine codon encoded by a GCG codon.

In some embodiments, the nucleic acid sequence encoding for DA-L4 100K further comprises one or more non-synonymous codons.

In some embodiments, the AAV production system further comprises a nucleic acid sequence encoding for DA-L4 100K wherein the DA-L4 100K has at least 80% identity with SEQ ID NO: 39, and wherein: (i) a tryptophan residue of DA-L4 100K is replaced with a TGA stop codon, a TAG stop codon, or a TAA stop codon; (ii) a glutamine residue of DA-L4 100K is replaced with a TAG stop codon or a TAA stop codon; or (iii) an arginine residue of DA-L4 100K is replaced with a TGA stop codon.

In some embodiments, the AAV production system comprises a nucleic acid sequence encoding for DA-VARNA, wherein the DA-VARNA comprises one or more non-synonymous codons and has at least 80% identity with SEQ ID NO: 13.

In some embodiments, the AAV production system further comprises a nucleic acid sequence encoding for DA-VARNA, wherein the DA-VARNA comprises one or more premature terminator sequences and has at least 80% identity with SEQ ID NO: 13.

In some embodiments, an inducible promoter is operably linked to the nucleic acid sequence encoding for one or more of: Rep52, DA-Rep52, Rep40, or DA-Rep40; Rep78, DA-Rep78, Rep68, or DA-Rep68; E2A or DA-E2A; E4ORF6 or DA-E4ORF6; VARNA or DA-VARNA; VP1 or DA-VP1; VP2 or DA-VP2; VP3 or DA-VP3; AAP or DA-AAP; MAAP or DA-MAAP; and L4 100K or DA-L4 100K.

In some embodiments, an inducible promoter is operably linked to the nucleic acid sequence encoding for the Base Editor.

In some embodiments, the engineered cell further comprises a nucleic acid sequence encoding for a transcriptional activator that is capable of binding to the inducible promoter.

In some embodiments, the transcriptional activator is selected from the group consisting of TetOn-3G, TetOn-V16, TetOff-Advanced, VanR-VP16, TtgR-VP16, PhlF-VP16, and the cumate cTA and rcTA.

In some embodiments, the transcriptional activator is TetOn 3G.

In some embodiments, the Base Editor comprises an Adenine Base Editor (ABE).

In some embodiments, the ABE is Cas9-ABE or Cas13-ABE.

In some embodiments, the Cas9-ABE is encoded for by an amino acid sequence having at least 80% identity with SEQ ID NO: 23 or 24.

In some embodiments, the Cas13-ABE is encoded for by an amino acid sequence having at least 80% identity with SEQ ID NO: 25 or 26.

In some embodiments, the Base Editor comprises an Cytosine Base Editor (CBE).

In some embodiments, the CBE is Cas9-CBE or Cas13-CBE.

In some embodiments, the Cas9-CBE is encoded for by an amino acid sequence having at least 80% identity with SEQ ID NO: 44 or 45.

In some embodiments, the engineered cell is a HEK293 or HeLa cell.

In some aspects, this application discloses a kit comprising the engineered cell described herein.

In some embodiments, the kit further comprises a polynucleotide comprising, from 5′ to 3′: (i) a nucleic acid sequence of a 5′ inverted terminal repeat; (ii) a multiple cloning site; and (iii) a nucleic acid sequence of a 3′ inverted terminal repeat.

In some embodiments, the polynucleotide is a plasmid or a vector.

In some aspects, this application discloses a method for AAV production, comprising contacting the engineered cell disclosed herein with a small molecule inducer that binds to the transcriptional activator.

In some embodiments, the small molecule inducer is selected from the group consisting of doxycycline, vanillate, phloretin, rapamycin, abscisic acid, gibberellic acid acetoxymethyl ester, and cumate.

In some aspects, this application discloses a method for AAV production, comprising expressing in the engineered cell described herein and one or more single guide RNA(s) capable of targeting the Base Editor to a mutated start codon, mutated non-synonymous codon, or a premature stop codon of DA-Rep52, DA-Rep40, DA-Rep78, DA-Rep68, DA-E2A, DA-E4ORF6, DA-VP1, DA-VP2, DA-VP3, DA-AAP, DA-MAAP, or DA-L4 100K, wherein the engineered cell comprising one or more polynucleotides encoding the one or more single guide RNA(s).

In some embodiments, the one or more polynucleotides encoding the one or more single guide RNA(s) are stably integrated.

In some aspects, this application discloses a method for AAV production, comprising contacting the engineered cell described herein with one or more single guide RNA(s) capable of targeting the Base Editor to a mutated start codon, mutated non-synonymous codon, or a premature stop codon of DA-Rep52, DA-Rep40, DA-Rep78, DA-Rep68, DA-E2A, DA-E4ORF6, DA-VP1, DA-VP2, DA-VP3, DA-AAP, DA-MAAP, or DA-L4 100K such that that engineered cell internalizes the one or more single guide RNA(s).

### BRIEF DESCRIPTION OF THE DRAWINGS

FIG. 1 is a plasmid schematic for transient transfection plasmids. A mutated start codon is made by replacing the wild-type start codon with an isoleucine (I), threonine (T), valine (V), or alanine (A) codon in the coding sequence of Rep, Cap, E2A, L4 100K, and/or E4ORF6. Alternatively, a non-synonymous mutation or premature stop codon mutation is made by replacing any wild-type codon with a codon encoding a residue that reduces the function of Rep, Cap, VARNA, E2A, L4 100K, and/or E4ORF6. A constitutively expressed ABE or CBE and single guide RNA repair these mutated codons during transfection to produce AAV. In the absence of the base editor or single guide RNA, no AAV is produced.

FIG. 2 is a plasmid schematic for stable integration of plasmids. Transposon IR/DRs, CTCF insulators, and an antibiotic resistance selection cassette flank the AAV payload, mutant Rep/Cap, and mutant helper genes. One or more mutated start codons, non-synonymous mutations, or premature stop codon mutations can be introduced to Rep, Cap, VARNA, E2A, L4 100K, and/or E4ORF6. The base editor is expressed by an inducible TRE promoter, with the rtTA (TetOn) gene fused to an antibiotic resistance gene on the same plasmid.

FIG. 3 depicts the percentage of fluorescent cells expressing mutated start codons or non-synonymous codons within EGFP or iRFP720, in the presence or absence of AncBE4max to restore fluorescence. All EGFP and iRFP720 mutants tested, except mutant Y198C in iRFP720, were able to diminish protein activity in the absence of a CBE. All remaining mutants, except P66T in EGFP, were able to be recovered with AncBE4max to varying degrees. Mutating a start codon with an MIT mutation still reduced activity, but not as much as the non-synonymous mutants tested in this assay.

FIG. 4 depicts combined start mutants of Rep, E2A, and E4 ORF6, with or without AncBE4max to restore viral titer. The mutants tested were able to diminish AAV titers in the absence of a targeting CBE guide RNA to levels comparable with the negative control (“Negative”). When the correct guides to target Rep, E2A, and E4 ORF6 were added, titers were restored to the level of unedited plasmid co-transfection (“Start Codon Mutations”).

### DETAILED DESCRIPTION OF CERTAIN EMBODIMENTS

AAV are a promising gene delivery modality for cell and gene therapy. The production of AAV normally entails transient transfection of plasmids into cell culture. However, stable integration of genes necessary to produce therapeutic AAV into the genome offers several advantages compared to traditional production via transient transfection. Since cells amplify the viral genes during their own cell division, large quantities of DNA and transfection reagent no longer need to be procured for the transfection process, reducing costs. Also, since the DNA is already within the nucleus, viral titers may be higher and more consistent due to minimal numbers of “untransfected” cells and reduced variation associated with transfection steps. The simpler production process also reduces room for error.

However, several genes required for adeno-associated viral (AAV) vector production have been demonstrated by others to be cytostatic or cytotoxic, namely Rep, E2A and E4. The cytotoxic and cytostatic nature of these proteins has hampered the development of stable AAV producer cell lines in the widely used HEK293 cell line, since the native expression of adenovirus E1 genes in HEK293 cells upregulates expression of these toxic genes. Cells stably transfected with these genes fail to survive selection steps or have silenced expression, resulting in an inability to produce relevant quantities of AAV.

### I. Adeno-Associated Virus Production Systems

In some aspects, the disclosure relates to adeno-associated virus (AAV) production systems. In some embodiments, AAV production systems allow for inducible control of a gene product(s) required for AAV production, including a product(s) that is cytotoxic or cytostatic to a cell. This inducible control can be mediated at the genomic level (i.e., inducible control of genomic modification) or at the translational level (i.e., inducible control of altered translation).

An AAV production system, as described herein, comprises one or more polynucleic acids collectively comprising: (a) an AAV production component and (b) an activity control component.

As used herein, the term “AAV production component” refers to one or more polynucleic acids that collectively encode the gene products required for generation of AAV in a recombinant host cell, wherein at least one gene required for AAV production is modified to comprise a mutation that decreases the activity of the gene required for AAV production. In some embodiments, the mutation results in a mutated start codon. In some embodiments, the mutation results in a non-synonymous amino acid substitution. In some embodiments, the mutation results in a premature stop codon.

In some embodiments, the AAV production component comprises one or more polynucleotides that collectively encode the gene products required to generate an AAV vector in a recombinant host cell. Exemplary AAV gene products include Rep52, Rep40, Rep78, Rep68, E2A, E4ORF6, VARNA, CAP (VP1, VP2, VP3), AAP, and MAAP. The Rep gene products (comprising Rep52, Rep40, Rep78 and Rep68) are involved in AAV genome replication. The E2A gene product is involved in aiding DNA synthesis processivity during AAV replication. The E4ORF6 gene product supports AAV replication. The VARNA gene product plays a role in regulating translation. The CAP gene products (comprising VP1, VP2, VP3) encode viral capsid proteins. The AAP gene product plays a role in capsid assembly. The MAAP gene product supports viral secretion. In some embodiments, an AAV component comprises one or more polynucleotides that collectively encode the gene products: Rep52 or Rep40; Rep78 or Rep68; E2A; E4ORF6; VARNA; VP1; VP2; VP3; AAP, and MAAP. In some embodiments, a AAV component comprises one or more polynucleotides that collectively encode the gene products: Rep52, Rep40, Rep78, Rep68, E2A, E4ORF6, VARNA, VP1, VP2, VP3, AAP, and MAAP.

In some embodiments, the AAV production component comprises a heterologous polynucleic acid comprising a nucleic acid sequence encoding for a gene product(s) required for AAV production (e.g., a product(s) that is cytotoxic or cytostatic to the cell, such as Rep, E2A and/or E4), wherein the gene product(s) is modified to comprise a mutation that decreases the activity of the gene required for AAV production. In some embodiments, the mutation results in a mutated start codon. In some embodiments, the mutation causes a codon change that results in a non-synonymous amino acid substitution. In some embodiments, the mutation results in a premature stop codon.

In some embodiments, the heterologous polynucleic acid comprising a nucleic acid sequence encoding for a gene product(s) required for AAV production comprises at least 1 mutation (e.g., at least 1, at least 2, at least 3, at least 4, at least 5, at least 6, at least 7, at least 8, at least 9, or at least 10 mutations). In some embodiments, the heterologous polynucleic acid comprising a nucleic acid sequence encoding for a gene product(s) required for AAV production comprises 1, 2, 3, 4, 5, 6, 7, 8, 9, or 10 mutation(s). In some embodiments, the heterologous polynucleic acid comprising a nucleic acid sequence encoding for a gene product(s) required for AAV production comprises 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 mutations. In some embodiments, any codon within the heterologous polynucleic acid comprising a nucleic acid sequence encoding for a gene product(s) required for AAV production can be mutated.

As used herein, the term “mutated start codon” refers to a start codon removed from the coding sequence of a gene (e.g., ATG, CTG or ACG) by mutating one or more nucleic acid residues in the start codon. For example, an ATG start codon may be replaced with an ATA codon, an ACG codon, a GTG codon or a GCG codon. In another example, a CTG start codon may be replaced with a CTA codon, a TTG codon or a CCG codon. In another example, an ACG start codon may be replaced with an ACA codon or a GCG codon.

In some embodiments, the heterologous polynucleic acid comprising a nucleic acid sequence encoding for a gene product(s) required for AAV production comprises at least 1 mutated start codon (e.g., at least 1, at least 2, at least 3, at least 4, at least 5, at least 6, at least 7, at least 8, at least 9, or at least 10 mutated start codons). In some embodiments, the heterologous polynucleic acid comprising a nucleic acid sequence encoding for a gene product(s) required for AAV production comprises 1, 2, 3, 4, 5, 6, 7, 8, 9, or 10 mutated start codon(s). In some embodiments, the heterologous polynucleic acid comprising a nucleic acid sequence encoding for a gene product(s) required for AAV production comprises 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 mutated start codon(s). In some embodiments, any codon within the heterologous polynucleic acid comprising a nucleic acid sequence encoding for a gene product(s) required for AAV production can be modified to a mutated start codon.

A codon mutation that results in a “non-synonymous amino acid substitution,” as described herein, is one that alters the amino acid encoded by the codon. As those in the art will appreciate, a subset of codon mutations may not alter the amino acid encoded by the codon. For example, both the GCA codon and the GCG codon correspond to adenine (A). Thus, a GCA to GCG mutation would not result in a non-synonymous amino acid substitution. In contrast, a GCT to GTT mutation would result in a non-synonymous amino acid substitution (i.e., A to V).

In some embodiments, the heterologous polynucleic acid comprising a nucleic acid sequence encoding for a gene product(s) required for AAV production comprises at least 1 mutated codon (e.g., at least 1, at least 2, at least 3, at least 4, at least 5, at least 6, at least 7, at least 8, at least 9, or at least 10 mutated codons) that results in a non-synonymous amino acid substitution. In some embodiments, the heterologous polynucleic acid comprising a nucleic acid sequence encoding for a gene product(s) required for AAV production comprises 1, 2, 3, 4, 5, 6, 7, 8, 9, or 10 mutated codon(s) that result in non-synonymous amino acid substitutions. In some embodiments, the heterologous polynucleic acid comprising a nucleic acid sequence encoding for a gene product(s) required for AAV production comprises 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 mutated codon(s) that results in non-synonymous amino acid substitutions. In some embodiments, any codon within the heterologous polynucleic acid comprising a nucleic acid sequence encoding for a gene product(s) required for AAV production can be modified to a mutated codon that results in a non-synonymous amino acid substitution.

As used herein, the term “premature stop codon” refers to a stop codon added to the coding sequence of a gene by mutating one or more nucleic acid residues in the coding sequence such that the sequence of a given codon becomes TAG, TAA, or TGA.

In some embodiments, the heterologous polynucleic acid comprising a nucleic acid sequence encoding for a gene product(s) required for AAV production comprises at least 1 premature stop codon (e.g., at least 1, at least 2, at least 3, at least 4, at least 5, at least 6, at least 7, at least 8, at least 9, or at least 10 premature stop codons). In some embodiments, the heterologous polynucleic acid comprising a nucleic acid sequence encoding for a gene product(s) required for AAV production comprises 1, 2, 3, 4, 5, 6, 7, 8, 9, or 10 premature stop codon(s). In some embodiments, the heterologous polynucleic acid comprising a nucleic acid sequence encoding for a gene product(s) required for AAV production comprises 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 premature stop codon(s). In some embodiments, any codon within the heterologous polynucleic acid comprising a nucleic acid sequence encoding for a gene product(s) required for AAV production can be modified to a premature stop codon.

In some embodiments, the AAV production component is (i.e., the gene products of the AAV component are) encoded on a single polynucleic acid. In other embodiments, multiple polynucleic acids collectively comprise the AAV component (i.e., at least two of the gene products of the AAV component are encoded on different polynucleic acids). For example, an AAV component may comprise at least 2, at least 3, at least 4, or at least 5 polynucleic acids. In some embodiments, an AAV component comprises 2, 3, 4, or 5 polynucleic acids.

As used herein, the term “activity control component” refers to one or more polynucleic acids that collectively encode factors required for inducing production of fully functional AAV production gene products (with functionality equivalent to or exceeding wild type levels). Exemplary activity control components described herein include a Base Editor system.

In some embodiments, the activity control component comprises a Base Editor (e.g., an ABE or CBE) capable of correcting one or more mutations in a gene required for AAV production. In some embodiments, the activity control component comprises a Base Editor (e.g., an ABE or CBE) capable of correcting 1, 2, 3, 4, 5, 6, 7, 8, 9, 10, or 20 mutations in a gene required for AAV production. In some embodiments, the activity control component comprises a Base Editor (e.g., an ABE or CBE) capable of editing a mutated start codon such that it encodes a start codon (i.e., ATG). In some embodiments, the activity control component comprises a Base Editor (e.g., an ABE or CBE) capable of editing a non-synonymous codon(s) such that it encodes the original wild-type codon. In some embodiments, the activity control component comprises a Base Editor (e.g., an ABE or CBE) capable of editing a premature stop codon (i.e., TAA, TAG, or TGA) such that it encodes the original wild-type codon.

In some embodiments, the activity control component is encoded on a single polynucleic acid. In some embodiments, multiple polynucleic acids collectively comprise the activity control component. For example, an activity control component may comprise at least 2, at least 3, at least 4, or at least 5 polynucleic acids. In some embodiments, an activity control component comprises 2, 3, 4, 5, 6, 7, 8, 9, 10, 11, 12, 13, 14, 15, 16, 17, 18, 19, or 20 polynucleic acids.

As used herein, the term “promoter” refers to a nucleic acid sequence that is capable of being bound by a protein to initiate transcription of RNA from DNA. A promoter may be a constitutive promoter (i.e., an unregulated promoter that allows for continual transcription). Examples of constitutive promoters are known in the art and include, but are not limited to, cytomegalovirus (CMV) promoters, elongation factor 1 α (EF1α) promoters, simian vacuolating virus 40 (SV40) promoters, ubiquitin-C (UBC) promoters, U6 promoters, and phosphoglycerate kinase (PGK) promoters. See e.g., Ferreira et al., Tuning gene expression with synthetic upstream open reading frames. Proc. Natl. Acad. Sci. U.S.A. 2013 July; 110(28): 11284-89; Pub. No.: US 2014/377861 A1—the entireties of which are incorporated herein by reference particularly for the disclosure relating to constitutive promoters. Alternatively, a promoter may be an inducible promoter (i.e., only activates transcription under specific circumstances). An inducible promoter may be a chemically inducible promoter, a temperature inducible promoter, or a light inducible promoter. Examples of inducible promoters are known in the art and include, but are not limited to, tetracycline/doxycycline inducible promoters, cumate inducible promoters, ABA inducible promoters, CRY2-CIB1 inducible promoters, DAPG inducible promoters, and mifepristone inducible promoters. See e.g., Stanton et al., ACS Synth. Biol. 2014 Dec. 19; 3(12): 880-91; Liang et al., Sci. Signal. 2011 Mar. 15; 4(164): rs2; U.S. Pat. Nos. 7,745,592 B2; 7,935,788 B2—the entireties of which are incorporated herein by reference particularly for the disclosure relating to inducible promoters.

A. Mutations that Decrease AAV Gene Product Activity

In some embodiments, the AAV production component comprises a heterologous polynucleic acid comprising a nucleic acid sequence encoding for a gene product(s) required for AAV production, wherein the gene product(s) is modified to comprise one or more mutations that decrease the function of the gene product. In some embodiments, the polynucleic acid encoding for the gene product(s) required for AAV production may comprise 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 mutations that decrease the function of the gene product.

In some embodiments, the one or more mutations decrease the activity of the gene product required for AAV production by at least 10% (e.g. at least 10%, at least 20%, at least 30%, at least 40%, at least 50%, at least 60%, at least 70%, at least 80%, at least 90%, at least 95%, at least 98%, or at least 99%) compared to the wild-type gene product. In some embodiments, the one or more mutations decrease the activity of the gene product required for AAV production by 10%-20%, 10%-30%, 10%, 50%, 10%-70%, 10%-90%, 10%-99%, 30%-50%, 30%-70%, 30%-90%, 30%-99%, 50%-70%, 50%-90%, 50%-99%, 70%-90%, or 70%-99%. In some embodiments, the one or more mutations in the gene required for AAV production result in loss of function of the gene product. In some embodiments, the one or more mutations decrease AAV production in a cell by at least 1-fold (e.g. at least 1-fold, at least 2-fold, at least 5-fold, at least 10-fold, at least 50-fold, at least 100-fold, at least 500-fold, at least 1000-fold, at least 10000-fold. In some embodiments, the one or more mutations decrease AAV production in a cell 1-2, 1-5, 1-10, 1-50, 1-100, 1-1000, 5-10, 5-50, 5-100, 5-1000, 10-20, 10-50, 10-100, 10-1000, 10-10000, 100-1000, or 100-10000 fold.

In some embodiments, the one or more mutations are selected from the codon mutations in Table 1 and Table 2. In some embodiments, the one or more mutations comprise codon mutations that result in an amino acid of different classification being encoded compared to the wild-type encoded amino acid. In some embodiments, the different classifications of amino acids are Positively Charged: arginine, histidine, and lysine; Negatively Charged: aspartic acid and glutamic acid; Polar: serine, threonine, cysteine, tyrosine, asparagine, and glutamine; Nonpolar: glycine, alanine, valine, leucine, isoleucine, methionine, tryptophan, phenylalanine, or proline. In some embodiments, one or more amino acid codons for a positively charged amino acid(s) is replaced with a codon for a negatively charged, nonpolar, or polar amino acid. In some embodiments, one or more amino acid codon(s) for a negatively charged amino acid is replaced with a codon for a positively charged, nonpolar, or polar amino acid. In some embodiments, one or more amino acid codon(s) for a polar amino acid is replaced with a codon for a negatively charged, positively charged, or polar amino acid. In some embodiments, one or more amino acid codon(s) for a nonpolar amino acid is replaced with a codon for a negatively charged, nonpolar, or positively charged amino acid.

In some embodiments, the AAV production component comprises a heterologous polynucleic acid comprising a nucleic acid sequence encoding for a gene product(s) required for AAV production, wherein the gene product(s) is modified to comprise a mutated start codon(s). In some embodiments, the polynucleic acid encoding for the gene product(s) may comprise an isoleucine codon (e.g., ATA), a threonine codon (e.g., ACG), a valine codon (e.g., GTG), or an alanine codon (e.g., GCG) at a position corresponding to a methionine codon. In some embodiments, the polynucleic acid encoding for the gene product(s) may comprise one or more mutated start codon(s) (e.g. 1, 2, 3, 4, 5, 6, 7, 8, 9, or 10) at a position corresponding to a methionine codon. In some embodiments, the polynucleic acid encoding for the gene product(s) may comprise 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 mutated start codon(s) at a position(s) corresponding to a methionine codon.

In some embodiments, one or more start codons are removed by mutating ATG to an isoleucine codon (e.g., ATA), a threonine codon (e.g., ACG), a valine codon (e.g., GTG), or an alanine codon (e.g., GCG).

In some embodiments, the AAV production component comprises a heterologous polynucleic acid comprising a nucleic acid sequence encoding for a gene product(s) required for AAV production, wherein the gene product(s) is modified to comprise a premature stop codon(s). In some embodiments, the polynucleic acid encoding for the gene product(s) may comprise a premature stop codon at a position corresponding to a tryptophan codon, a glutamine codon or an arginine codon. In some embodiments, the polynucleic acid encoding for the gene product(s) may comprise one or more premature stop codon(s) (e.g. 1, 2, 3, 4, 5, 6, 7, 8, 9, or 10) at a position corresponding to a tryptophan codon, a glutamine codon or an arginine codon. In some embodiments, the polynucleic acid encoding for the gene product(s) may comprise 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 premature stop codon(s) at a position(s) corresponding to tryptophan codon a glutamine codon or an arginine codon.

In some embodiments, one or more stop codon mutations are inserted by mutating one or more tryptophan codon(s) on the sense DNA strand, or one or more glutamine and/or arginine codon(s) on the antisense DNA strand to premature stop codons.

The modifier “DA” as used herein, refers to a gene comprising one or more mutations that decrease the activity of the product of the gene (e.g., a mutated start codon(s) and/or a non-synonymous or premature stop codon(s)).

In some embodiments, the AAV production component comprises: a nucleic acid sequence encoding for DA-Rep52 operably linked to a promoter (constitutive or inducible, as described herein); a nucleic acid sequence encoding for DA-Rep40 operably linked to a nucleic acid sequence of a promoter (constitutive or inducible, as described herein); a nucleic acid sequence encoding for DA-Rep78 operably linked to a nucleic acid sequence of a promoter (constitutive or inducible, as described herein); a nucleic acid sequence encoding for DA-Rep68 operably linked to a nucleic acid sequence of a promoter (constitutive or inducible); a nucleic acid sequence encoding for DA-Rep78+52 operably linked to a nucleic acid sequence of a promoter (constitutive or inducible, as described herein); a nucleic acid sequence encoding for DA-Rep operably linked to a nucleic acid sequence of a promoter (constitutive or inducible, as described herein); a nucleic acid sequence encoding for DA-E2A operably linked to a nucleic acid sequence of a promoter (constitutive or inducible, as described herein); a nucleic acid sequence encoding for DA-E4ORF6 operably linked to a nucleic acid sequence of a promoter (constitutive or inducible, as described herein); a nucleic acid sequence encoding for DA-VP1 operably linked to a promoter (constitutive or inducible, as described herein); a nucleic acid sequence encoding for DA-VP2 operably linked to a promoter (constitutive or inducible, as described herein); a nucleic acid sequence encoding for DA-VP3 operably linked to a promoter (constitutive or inducible, as described herein); a nucleic acid sequence encoding for DA-VP operably linked to a promoter (constitutive or inducible, as described herein); a nucleic acid sequence encoding for DA-AAP operably linked to a promoter (constitutive or inducible, as described herein); a nucleic acid sequence encoding for DA-MAAP operably linked to a promoter (constitutive or inducible, as described herein); a nucleic acid sequence encoding for DA-L4 100K operably linked to a promoter (constitutive or inducible, as described herein); or any combination thereof.

In some embodiments, the nucleic acid sequences encoding DA-E2A, DA-E4ORF6, DA-Rep52, DA-Rep40, DA-Rep78, DA-Rep68, DA-Rep, DA-VP1, DA-VP2, DA-VP3, DA-VP, DA-AAP, DA-MAAP, and DA-L4 100K further comprise one or more mutations to introduce a PAM sequence. In some embodiments, the nucleic acid sequences encoding DA-E2A, DA-E4ORF6, DA-Rep52, DA-Rep40, DA-Rep78, DA-Rep68, DA-Rep, DA-VP1, DA-VP2, DA-VP3, DA-VP, DA-AAP, DA-MAAP, and DA-L4 100K further comprise one or more silent mutations to introduce a PAM sequence. In some embodiments, the PAM sequence is introduced near the mutation(s) to introduce a PAM sequence for a DNA Base editor (e.g. Cas9 containing ABEs or CBEs). In some embodiments, the PAM sequence is introduced 1, 2, 3, 4, 5, 6, 7, 8, 9, 10, 11, 12, 13, 14, 15, 16, 17, 18, 19, or 20 nucleotides upstream of the target editing site. In some embodiments, the PAM sequence is introduced within 1, 2, 3, 4, 5, 6, 7, 8, 9, 10, 11, 12, 13, 14, 15, 16, 17, 18, 19, and 20 nucleotides downstream of the targeting editing site. In some embodiments, the PAM sequence is introduced within 10-17 or 13-16 nucleotide (upstream or downstream) of the target editing site. In some embodiments, one or more silent mutations are made to reduce off-target base editing within the Base Editor window. In some embodiments, one or more silent mutations are made to reduce transcription from alternative start sites. In some embodiments, one or more conservative mutations are made to reduce transcription from alternative start sites.

In some embodiments, the AAV production component comprises a nucleic acid sequence encoding for DA-Rep52 operably linked to a nucleic acid sequence of a promoter (constitutive or inducible, as described herein). In some embodiments, the DA-Rep52 nucleic acid sequence encoding an amino acid sequence is operably linked to a p19 promoter. The term “DA-Rep52” refers to a nucleic acid sequence encoding a polypeptide comprising at least 80% (e.g., at least 80%, at least 85%, at least 90%, at least 95%, or at least 99%) identity to the amino acid sequence of SEQ ID NO: 6 comprising at least one mutation that decreases the activity of Rep52 (as described above) (e.g., a mutated start codon, a non-synonymous codon, and/or a premature stop codon). In some embodiments, DA-Rep52 comprises a nucleic acid sequence that is modified such that it has a mutated start codon. In some embodiments, the start codon (ATG) has been replaced with an isoleucine codon (e.g., ATA), a threonine codon (e.g., ACG), a valine codon (e.g., GTG), or an alanine codon (e.g., GCG). In some embodiments, DA-Rep52 comprises a nucleic acid sequence that is modified such that it has a non-synonymous codon. In some embodiments, DA-Rep52 comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, the AAV production component comprises a nucleic acid sequence encoding for DA-Rep40 operably linked to a nucleic acid sequence of a promoter (constitutive or inducible, as described herein). In some embodiments, the DA-Rep40 nucleic acid sequence is operably linked to a p19 promoter. The term “DA-Rep40” refers to a nucleic acid sequence encoding a polypeptide comprising at least 80% (e.g., at least 80%, at least 85%, at least 90%, at least 95%, or at least 99%) identity to the amino acid sequence of SEQ ID NO: 7 comprising at least one mutation that decreases the activity of Rep40 (as described above) (e.g., a mutated start codon, a non-synonymous codon, and/or a premature stop codon). In some embodiments, DA-Rep40 comprises a nucleic acid sequence that is modified such that it has a mutated start codon. In some embodiments, the start codon (ATG) has been replaced with an isoleucine codon (e.g., ATA), a threonine codon (e.g., ACG), a valine codon (e.g., GTG), or an alanine codon (e.g., GCG). In some embodiments, DA-Rep40 comprises a nucleic acid sequence that is modified such that it has a non-synonymous codon. In some embodiments, DA-Rep40 comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, the AAV production component comprises a nucleic acid sequence encoding for DA-Rep78 operably linked to a nucleic acid sequence of a promoter (constitutive or inducible, as described herein). In some embodiments, the DA-Rep78 nucleic acid sequence encoding is operably linked to a p5 promoter. The term “DA-Rep78” refers to a nucleic acid sequence encoding a polypeptide comprising at least 80% (e.g., at least 80%, at least 85%, at least 90%, at least 95%, or at least 99%) identity to the amino acid sequence of SEQ ID NO: 8 comprising at least one mutation that decreases the activity of Rep78 (as described above) (e.g., a mutated start codon, a non-synonymous codon, and/or a premature stop codon). In some embodiments, DA-Rep78 comprises a nucleic acid sequence that is modified such that it has a mutated start codon. In some embodiments, the start codon (ATG) has been replaced with an isoleucine codon (e.g., ATA), a threonine codon (e.g., ACG), a valine codon (e.g., GTG), or an alanine codon (e.g., GCG). In some embodiments, DA-Rep78 comprises a nucleic acid sequence that is modified such that it has a non-synonymous codon. In some embodiments, DA-Rep78 comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, the AAV production component comprises a nucleic acid sequence encoding for DA-Rep68 operably linked to a nucleic acid sequence of a promoter (constitutive or inducible, as described herein). In some embodiments, the DA-Rep68 nucleic acid sequence is operably linked to a p5 promoter. The term “DA-Rep68” refers to a nucleic acid sequence encoding a polypeptide comprising at least 80% (e.g., at least 80%, at least 85%, at least 90%, at least 95%, or at least 99%) identity to the amino acid sequence of SEQ ID NO: 9 comprising at least one mutation that decreases the activity of Rep68 (as described above) (e.g., a mutated start codon, a non-synonymous codon, and/or a premature stop codon). In some embodiments, DA-Rep68 comprises a nucleic acid sequence that is modified such that it has a mutated start codon. In some embodiments, the start codon (ATG) has been replaced with an isoleucine codon (e.g., ATA), a threonine codon (e.g., ACG), a valine codon (e.g., GTG), or an alanine codon (e.g., GCG). In some embodiments, DA-Rep68 comprises a nucleic acid sequence that is modified such that it has a non-synonymous codon. In some embodiments, DA-Rep68 comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, the AAV production component comprises a nucleic acid sequence encoding for DA-Rep operably linked to a nucleic acid sequence of a promoter (constitutive or inducible, as described herein). In some embodiments, the DA-Rep nucleic acid sequence is operably linked to a p5 and p19 promoter. The term “DA-Rep” refers to a nucleic acid sequence comprising at least 80% (e.g., at least 80%, at least 85%, at least 90%, at least 95%, or at least 99%) identity to sequence of SEQ ID NO: 38 comprising at least one mutation that decreases the activity of Rep (as described above) (e.g., a mutated start codon, a non-synonymous codon, and/or a premature stop codon). In some embodiments, DA-Rep comprises a nucleic acid sequence that is modified such that it has a mutated start codon. In some embodiments, the start codon (ATG) has been replaced with an isoleucine codon (e.g., ATA), a threonine codon (e.g., ACG), a valine codon (e.g., GTG), or an alanine codon (e.g., GCG). In some embodiments, DA-Rep comprises a nucleic acid sequence that is modified such that it has a non-synonymous codon. In some embodiments, DA-Rep comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, the AAV production component comprises a nucleic acid sequence encoding for DA-E2A operably linked to a nucleic acid sequence of a promoter (constitutive or inducible, as described herein). In some embodiments, the DA-E2A nucleic acid sequence is operably linked to an E2 promoter. The term “DA-E2A” refers to a nucleic acid sequence encoding a polypeptide comprising at least 80% (e.g., at least 80%, at least 85%, at least 90%, at least 95%, or at least 99%) identity to the amino acid sequence of SEQ ID NO: 10 comprising at least one mutation that decreases the activity of E2A (as described above) (e.g., a mutated start codon, a non-synonymous codon, and/or a premature stop codon). In some embodiments, DA-E2A comprises a nucleic acid sequence that is modified such that it has a mutated start codon. In some embodiments, the start codon (ATG) has been replaced with an isoleucine codon (e.g., ATA), a threonine codon (e.g., ACG), a valine codon (e.g., GTG), or an alanine codon (e.g., GCG). In some embodiments, DA-E2A comprises a nucleic acid sequence that is modified such that it has a non-synonymous codon. In some embodiments, DA-E2A comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, the AAV production component comprises a nucleic acid sequence encoding for DA-E4ORF6 operably linked to a nucleic acid sequence of a promoter (constitutive or inducible, as described herein). In some embodiments, the DA-E4ORF6 nucleic acid sequence is operably linked to an E4 promoter. The term “DA-E4ORF6” refers to a nucleic acid sequence encoding a polypeptide comprising at least 80% (e.g., at least 80%, at least 85%, at least 90%, at least 95%, or at least 99%) identity to the amino acid sequence of SEQ ID NO: 11 comprising at least one mutation that decreases the activity of E4ORF6 (as described above) (e.g., a mutated start codon, a non-synonymous codon, and/or a premature stop codon). In some embodiments, DA-E4ORF6 comprises a nucleic acid sequence that is modified such that it has a mutated start codon. In some embodiments, the start codon (ATG) has been replaced with an isoleucine codon (e.g., ATA), a threonine codon (e.g., ACG), a valine codon (e.g., GTG), or an alanine codon (e.g., GCG). In some embodiments, DA-E4ORF6 comprises a nucleic acid sequence that is modified such that it has a non-synonymous codon. In some embodiments, DA-E4ORF6 comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, the AAV production component comprises a nucleic acid sequence encoding for DA-AAP operably linked to a nucleic acid sequence of a promoter (constitutive or inducible, as described herein). In some embodiments, the DA-AAP nucleic acid sequence encoding an amino acid sequence is operably linked to a p40 promoter. The term “DA-AAP” refers to a nucleic acid sequence encoding a polypeptide comprising at least 80% (e.g., at least 80%, at least 85%, at least 90%, at least 95%, or at least 99%) identity to the amino acid sequence of SEQ ID NO: 17 comprising at least one mutation that decreases the activity of AAP (as described above) (e.g., a mutated start codon, a non-synonymous codon, and/or a premature stop codon). In some embodiments, DA-AAP comprises a nucleic acid sequence that is modified such that it has a mutated start codon. In some embodiments, the CTG start codon has been replaced with a leucine codon (e.g., CTA or TTG) or a proline codon (e.g., CCG). In some embodiments, DA-AAP comprises a nucleic acid sequence that is modified such that it has a non-synonymous codon. In some embodiments, DA-AAP comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, the AAV production component comprises a nucleic acid sequence encoding for DA-MAAP operably linked to a nucleic acid sequence of a promoter (constitutive or inducible, as described herein). In some embodiments, the DA-MAAP nucleic acid sequence encoding an amino acid sequence is operably linked to a p40 promoter. The term “DA-MAAP” refers to a nucleic acid sequence encoding a polypeptide comprising at least 80% (e.g., at least 80%, at least 85%, at least 90%, at least 95%, or at least 99%) identity to the amino acid sequence of SEQ ID NO: 43 comprising at least one mutation that decreases the activity of MAAP (as described above) (e.g., a mutated start codon, a non-synonymous codon, and/or a premature stop codon). In some embodiments, DA-MAAP comprises a nucleic acid sequence that is modified such that it has a mutated start codon. In some embodiments, the CTG start codon has been replaced with a leucine codon (e.g., CTA or TTG) or a proline codon (e.g., CCG). In some embodiments, DA-MAAP comprises a nucleic acid sequence that is modified such that it has a non-synonymous codon. In some embodiments, DA-MAAP comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, the AAV production component comprises a nucleic acid sequence encoding for DA-L4 100K operably linked to a nucleic acid sequence of a promoter (constitutive or inducible, as described herein). In some embodiments, the DA-L4 100K nucleic acid sequence encoding an amino acid sequence is operably linked to a L4 promoter. The term “DA-L4 100K” refers to a nucleic acid sequence encoding a polypeptide comprising at least 80% (e.g., at least 80%, at least 85%, at least 90%, at least 95%, or at least 99%) identity to the amino acid sequence of SEQ ID NO: 39 comprising at least one mutation that decreases the activity of L4 100K (as described above) (e.g., a mutated start codon, a non-synonymous codon, and/or a premature stop codon). In some embodiments, DA-L4 100K comprises a nucleic acid sequence that is modified such that it has a mutated start codon. In some embodiments, the start codon (ATG) has been replaced with an isoleucine codon (e.g., ATA), a threonine codon (e.g., ACG), a valine codon (e.g., GTG), or an alanine codon (e.g., GCG). In some embodiments, DA-L4 100K comprises a nucleic acid sequence that is modified such that it has a non-synonymous codon. In some embodiments, DA-L4 100K comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, DA-VARNA comprises a nucleic acid sequence comprising at least 80% (e.g., at least 80%, at least 85%, at least 90%, at least 95%, or at least 99%) identity to the nucleotide sequence of SEQ ID NO: 13 further comprising a mutation that renders VARNA inactive. In some embodiments, DA-VARNA comprises a nucleic acid sequence comprising a non-coding RNA sequence of SEQ ID NO: 13 further comprising at least one mutation that renders VARNA inactive. In some embodiments, DA-VARNA comprises a nucleic acid sequence comprising a non-coding RNA sequence of SEQ ID NO: 13 further comprising at least one premature terminator (e.g., TTTT) that renders VARNA inactive.

In some embodiments, the AAV production component comprises a nucleic acid sequence encoding for DA-VP1 operably linked to a nucleic acid sequence of a promoter (constitutive or inducible, as described herein). In some embodiments, the DA-VP1 nucleic acid sequence is operably linked to a p40 promoter. The term “DA-VP1” refers to a nucleic acid sequence encoding a polypeptide comprising at least 80% (e.g., at least 80%, at least 85%, at least 90%, at least 95%, or at least 99%) identity to the amino acid sequence of SEQ ID NO: 14 comprising at least one mutation that decreases the activity of VP1 (as described above) (e.g., a mutated start codon, a non-synonymous codon, and/or a premature stop codon). In some embodiments, DA-VP1 comprises a nucleic acid sequence that is modified such that it has a mutated start codon. In some embodiments, the start codon (ATG) has been replaced with an isoleucine codon (e.g., ATA), a threonine codon (e.g., ACG), a valine codon (e.g., GTG), or an alanine codon (e.g., GCG). In some embodiments, DA-VP1 comprises a nucleic acid sequence that is modified such that it has a non-synonymous codon. In some embodiments, DA-VP1 comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, the AAV production component comprises a nucleic acid sequence encoding for DA-VP2 operably linked to a nucleic acid sequence of a promoter (constitutive or inducible, as described herein). In some embodiments, the DA-VP2 nucleic acid sequence is operably linked to a p40 promoter. The term “DA-VP2” refers to a nucleic acid sequence encoding a polypeptide comprising at least 80% (e.g., at least 80%, at least 85%, at least 90%, at least 95%, or at least 99%) identity to the amino acid sequence of SEQ ID NO: 15 comprising at least one mutation that decreases the activity of VP2 (as described above) (e.g., a mutated start codon, a non-synonymous codon, and/or a premature stop codon). In some embodiments, DA-VP2 comprises a nucleic acid sequence that is modified such that it has a mutated start codon. In some embodiments, the ACG start codon has been replaced with a threonine codon (e.g., ACA) or an alanine codon (e.g., GCG). In some embodiments, DA-VP2 comprises a nucleic acid sequence that is modified such that it has a non-synonymous codon. In some embodiments, DA-VP2 comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, the AAV production component comprises a nucleic acid sequence encoding for DA-VP3 operably linked to a nucleic acid sequence of a promoter (constitutive or inducible, as described herein). In some embodiments, the DA-VP3 nucleic acid sequence is operably linked to a p40 promoter. The term “DA-VP3” refers to a nucleic acid sequence encoding a polypeptide comprising at least 80% (e.g., at least 80%, at least 85%, at least 90%, at least 95%, or at least 99%) identity to the amino acid sequence of SEQ ID NO: 16 comprising at least one mutation that decreases the activity of VP3 (as described above) (e.g., a mutated start codon, a non-synonymous codon, and/or a premature stop codon). In some embodiments, DA-VP3 comprises a nucleic acid sequence that is modified such that it has a mutated start codon. In some embodiments, the start codon (ATG) has been replaced with an isoleucine codon (e.g., ATA), a threonine codon (e.g., ACG), a valine codon (e.g., GTG), or an alanine codon (e.g., GCG). In some embodiments, DA-VP3 comprises a nucleic acid sequence that is modified such that it has a non-synonymous codon. In some embodiments, DA-VP3 comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

In some embodiments, the AAV production component comprises a nucleic acid sequence encoding for DA-VP operably linked to a nucleic acid sequence of a promoter (constitutive or inducible, as described herein). In some embodiments, the DA-VP nucleic acid sequence is operably linked to a p40 promoter. The term “DA-VP” refers to a nucleic acid sequence comprising at least 80% (e.g., at least 80%, at least 85%, at least 90%, at least 95%, or at least 99%) identity SEQ ID NO: 40 comprising at least one mutation that decreases the activity of VP (as described above) (e.g., a mutated start codon, a non-synonymous codon, and/or a premature stop codon). In some embodiments, DA-VP comprises a nucleic acid sequence that is modified such that it has a mutated start codon. In some embodiments, the start codon (ATG) has been replaced with an isoleucine codon (e.g., ATA), a threonine codon (e.g., ACG), a valine codon (e.g., GTG), or an alanine codon (e.g., GCG). In some embodiments, DA-VP comprises a nucleic acid sequence that is modified such that it has a non-synonymous codon. In some embodiments, DA-VP comprises one or more non-silent mutations that are detrimental to the activity of VP as described in Ogden et al. Science. 2019 Nov. 29; 366 (6469): 1139-1143, which is incorporated by reference herein in its entirety. In some embodiments, DA-VP comprises a nucleic acid sequence that is modified such that it has a premature stop codon. In some embodiments, a tryptophan codon (e.g., TGG) has been replaced with a stop codon (e.g., TAA, TAG, or TGA), a glutamine codon (e.g., CAA or CAG) has been replaced with a stop codon (e.g., TAA or TAG), or an arginine codon (e.g., CGA) has been replaced with a stop codon (e.g., TGA).

### B. Transcriptional Activator

In some embodiments, the AAV production system further comprises a nucleic acid sequence encoding a transcriptional activator that binds to an inducible promoter (e.g., operably linked to a nucleic acid sequence encoding for a gene required for AAV production or a Base Editor).

In some embodiments, the transcriptional activator is selected from the group consisting of TetOn-3G, rtTA-VP16, TetOff-Advanced, VanR-VP16, TtgR-VP16, PhlF-VP16, and the cumate cTA and rcTA. In some embodiments, the transcriptional activator is a rtTA/TetOn variant selected from the group consisting of rtTA-V1, rtTA-V2, rtTA-V3, rtTA-V4, rtTA-V5, rtTA-V7, rtTA-V8, rtTA-V9, rtTA-V10, rtTA-V11, rtTA-V12, rtTA-V13, rtTA-V14, rtTA-V15, rtTA-V16, rtTA-V17, and rtTA-V18 as described in Das et al. Curr. Gene Therapy 2016; 16(3):156-67, which is incorporated by reference in its entirety.

In some embodiments, the transcriptional activator is a transcriptional activator having at least 80% identity (e.g., at least 80% identity, at least 85% identity, at least 90% identity, at least 95% identity, or at least 99% identity) with any one of SEQ ID NOs: 33-37 and 42.

In some embodiments, the nucleic acid sequence encoding the transcriptional activator fused to a selection marker.

In some embodiments, the transcriptional activator is operably linked to a promoter. In some embodiments, the transcriptional activation is operably linked to a constitutively active promoter. In some embodiments, the transcriptional activator is operably linked to its corresponding chemically inducible promoter. In a non-limiting example, a TetOn-3G transcriptional activator may be operably linked to a TRE promoter. In some embodiments, the transcriptional activation is operably linked to a hEF1a promoter. In some embodiments, the transcriptional activator, when exposed to a small molecule inducer, induces the expression of corresponding chemically inducible promoters within the engineered cell. In some embodiments, the small molecule inducer is selected from the group consisting of doxycycline, vanillate, phloretin, rapamycin, abscisic acid, gibberellic acid acetoxymethyl ester, and cumate.

### II. AAV Production Systems Comprising a Base Editor

In some embodiments, the AAV production system comprises one or more polynucleic acids encoding a Base Editor capable of correcting a mutation(s) in nucleic acid sequence encoding for a gene required for AAV production. In some embodiments, the Base Editor replaces a mutated start codon with a start codon. In some embodiments, the Base Editor replaces a non-synonymous amino acid codon with a wild-type amino acid codon. In some embodiments, the Base Editor replaces a premature stop codon with a wild-type codon. In some embodiments, the AAV production system further comprises a single guide RNA (sgRNA).

### A. Base Editor

As described herein, the term “Base Editor” refers to a protein or fusion protein capable of introducing single-nucleotide variants (SNVs) into DNA or RNA. Exemplary Base Editors include but are not limited to Cytosine Base Editors (CBE): BE1, BE2, HF2-BE2, BE3, HF-BE3, YE1-BE3, EE-BE3, YEE-BE3, VQR-BE3, EQR-BE3, VRER-BE3, SaKKHBE3, FNLS-BE3, RA-BE3, eA3A-HF1-BE3-2×UGI, eA3A-Hypa-BE3-2×UGI, hA3A-BE3, hA3B-BE3, hA3G-BE3, hAID-BE3, SaCas9-BE3, xCas9-BE3, ScCas9-BE3, SniperCas9-BE3, iSpyMac-BE3, Target-AID, Target-AID-NG, BE-PLUS, BE4, BE4-Gam, BE4-Max, AncBE4-Max, SaCas9BE4-Gam, evoBe4max, evoFERNY-BE4max, and Cas12a-BE; and Adenine Base Editors (ABE): ABE7.8, ABE9, ABE10, ABE.8.17, xCas9-ABE7.10, VQR-ABE, Sa (KKH)-ABE, ABEmax, ABE7.10max, ABE8e, PE1, PE2, PE3, ABE REPAIRv1, and ABE Repairv2, which are described in more detail in Porto, Elizabeth M., et al. Nature Reviews Drug Discovery 19.12 (2020): 839-859; Cox, David B T, et al. Science 358.6366 (2017): 1019-1027; Komor, Alexis C., et al. Science advances 3.8 (2017): eaao4774; and Gaudelli, Nicole M., et al. Nature biotechnology 38.7 (2020): 892-900; and Kantor A. et al. International Journal of Molecular Sciences 21.17 (2020): 6240 each of which is incorporated by reference in its entirety. In a non-limiting overview, a Base Editor is a fusion protein comprising a CRISPR Cas protein domain with a catalytically inactive exonuclease domain (e.g. dCas9 or dCas13) or a CRISPR Cas nickase protein domain (e.g. Cas9n) and one or more domains capable of modifying DNA (e.g. a CBE or an ABE). The Base Editor binds to a single guide RNA (sgRNA) that comprises a nucleic acid sequence that is complementary to a target DNA or RNA sequence (as described below). The targeting of the Base Editor to DNA or RNA is determined by the type of Cas protein used (Cas9 for DNA and Cas13 for RNA). Examples of codon altering mutations that can be made using Base Editors are exemplified in Table 1 and Table 2. In some embodiments, zinc-finger nucleases, transcriptional activator-like effector nucleases (TALENs), or Prime Editors may be used in the place of a Base Editor.

TABLE 1
Possible codon mutations that can be made with an ABE.
Corresponding amino acids (or stop codons; “*”) are
provide in a parentheticals following the stop codons.

| ABE sense strand | | | ABE antisense strand | | |
|---|---|---|---|---|---|
| (DNA & RNA editors) | | | (DNA editors) | | |
| Original | -> | Mutant | Original | -> | Mutant |
| TAA (*) | -> | TGA (*) | TAA (*) | -> | CAA (Q) |
| TAA (*) | -> | TAG (*) | TAG (*) | -> | CAG (Q) |
| TAG (*) | -> | TGG (W) | TGA (*) | -> | CGA (R) |
| TGA (*) | -> | TGG (W) | GCT (A) | -> | GCC (A) |
| GCA (A) | -> | GCG (A) | TGT (C) | -> | CGT (R) |
| GAT (D) | -> | GGT (G) | TGT (C) | -> | TGC (C) |
| GAC (D) | -> | GGC (G) | TGC (C) | -> | CGC (R) |
| GAA (E) | -> | GGA (G) | GAT (D) | -> | GAC (D) |
| GAA (E) | -> | GAG (E) | TTT (F) | -> | CTT (L) |
| GAG (E) | -> | GGG (G) | TTT (F) | -> | TCT (S) |
| GGA (G) | -> | GGG (G) | TTT (F) | -> | TTC (F) |
| CAT (H) | -> | CGT (R) | TTC (F) | -> | CTC (L) |
| CAC (H) | -> | CGC (R) | TTC (F) | -> | TCC (S) |
| ATT (I) | -> | GTT (V) | GGT (G) | -> | GGC (G) |
| ATC (I) | -> | GTC (V) | CAT (H) | -> | CAC (H) |
| ATA (I) | -> | GTA (V) | ATT (I) | -> | ACT (T) |
| ATA (I) | -> | ATG (M) | ATT (I) | -> | ATC (I) |
| AAA (K) | -> | GAA (E) | ATC (I) | -> | ACC (T) |
| AAA (K) | -> | AGA (R) | ATA (I) | -> | ACA (T) |
| AAA (K) | -> | AAG (K) | TTA (L) | -> | CTA (L) |
| AAG (K) | -> | GAG (E) | TTA (L) | -> | TCA (S) |
| AAG (K) | -> | AGG (R) | TTG (L) | -> | CTG (L) |
| TTA (L) | -> | TTG (L) | TTG (L) | -> | TCG (S) |
| CTA (L) | -> | CTG (L) | CTT (L) | -> | CCT (P) |
| ATG (M) | -> | GTG (V) | CTT (L) | -> | CTC (L) |
| AAT (N) | -> | GAT (D) | CTC (L) | -> | CCC (P) |
| AAT (N) | -> | AGT (S) | CTA (L) | -> | CCA (P) |
| AAC (N) | -> | GAC (D) | CTG (L) | -> | CCG (P) |
| AAC (N) | -> | AGC (S) | ATG (M) | -> | ACG (T) |
| CCA (P) | -> | CCG (P) | AAT (N) | -> | AAC (N) |
| CAA (Q) | -> | CGA (R) | CCT (P) | -> | CCC (P) |
| CAA (Q) | -> | CAG (Q) | CGT (R) | -> | CGC (R) |
| CAG (Q) | -> | CGG (R) | TCT (S) | -> | CCT (P) |
| CGA (R) | -> | CGG (R) | TCT (S) | -> | TCC (S) |
| AGA (R) | -> | GGA (G) | TCC (S) | -> | CCC (P) |
| AGA (R) | -> | AGG (R) | TCA (S) | -> | CCA (P) |
| AGG (R) | -> | GGG (G) | TCG (S) | -> | CCG (P) |
| TCA (S) | -> | TCG (S) | AGT (S) | -> | AGC (S) |
| AGT (S) | -> | GGT (G) | ACT (T) | -> | ACC (T) |
| AGC (S) | -> | GGC (G) | GTT (V) | -> | GCT (A) |
| ACT (T) | -> | GCT (A) | GTT (V) | -> | GTC (V) |
| ACC (T) | -> | GCC (A) | GTC (V) | -> | GCC (A) |
| ACA (T) | -> | GCA (A) | GTA (V) | -> | GCA (A) |
| ACA (T) | -> | ACG (T) | GTG (V) | -> | GCG (A) |
| ACG (T) | -> | GCG (A) | TGG (W) | -> | CGG (R) |
| GTA (V) | -> | GTG (V) | TAT (Y) | -> | CAT (H) |
| TAT (Y) | -> | TGT (C) | TAT (Y) | -> | TAC (Y) |
| TAC (Y) | -> | TGC (C) | TAC (Y) | -> | CAC (H) |

TABLE 2
Possible codon mutations that can be made with a CBE.
Corresponding amino acids (or stop codons; “*”) are
provide in a parentheticals following the stop codons.

| CBE Sense | | | CBE antisense | | |
|---|---|---|---|---|---|
| (DNA & RNA editors) | | | (DNA editors) | | |
| Original | -> | Mutant | Original | -> | Mutant |
| GCT (A) | -> | GTT (V) | TAG (*) | -> | TAA (*) |
| GCC (A) | -> | GTC (V) | TGA (*) | -> | TAA (*) |
| GCC (A) | -> | GCT (A) | GCT (A) | -> | ACT (T) |
| GCA (A) | -> | GTA (V) | GCC (A) | -> | ACC (T) |
| GCG (A) | -> | GTG (V) | GCA (A) | -> | ACA (T) |
| TGC (C) | -> | TGT (C) | GCG (A) | -> | ACG (T) |
| GAC (D) | -> | GAT (D) | GCG (A) | -> | GCA (A) |
| TTC (F) | -> | TTT (F) | TGT (C) | -> | TAT (Y) |
| GGC (G) | -> | GGT (G) | TGC (C) | -> | TAC (Y) |
| CAT (H) | -> | TAT (Y) | GAT (D) | -> | AAT (N) |
| CAC (H) | -> | TAC (Y) | GAC (D) | -> | AAC (N) |
| CAC (H) | -> | CAT (H) | GAA (E) | -> | AAA (K) |
| ATC (I) | -> | ATT (I) | GAG (E) | -> | AAG (K) |
| CTT (L) | -> | TTT (F) | GAG (E) | -> | GAA (E) |
| CTC (L) | -> | TTC (F) | GGT (G) | -> | AGT (S) |
| CTC (L) | -> | CTT (L) | GGT (G) | -> | GAT (D) |
| CTA (L) | -> | TTA (L) | GGC (G) | -> | AGC (S) |
| CTG (L) | -> | TTG (L) | GGC (G) | -> | GAC (D) |
| AAC (N) | -> | AAT (N) | GGA (G) | -> | AGA (R) |
| CCT (P) | -> | TCT (S) | GGA (G) | -> | GAA (E) |
| CCT (P) | -> | CTT (L) | GGG (G) | -> | AGG (R) |
| CCC (P) | -> | TCC (S) | GGG (G) | -> | GAG (E) |
| CCC (P) | -> | CTC (L) | GGG (G) | -> | GGA (G) |
| CCC (P) | -> | CCT (P) | AAG (K) | -> | AAA (K) |
| CCA (P) | -> | TCA (S) | TTG (L) | -> | TTA (L) |
| CCA (P) | -> | CTA (L) | CTG (L) | -> | CTA (L) |
| CCG (P) | -> | TCG (S) | ATG (M) | -> | ATA (I) |
| CCG (P) | -> | CTG (L) | CCG (P) | -> | CCA (P) |
| CAA (Q) | -> | TAA (*) | CAG (Q) | -> | CAA (Q) |
| CAG (Q) | -> | TAG (*) | CGT (R) | -> | CAT (H) |
| CGT (R) | -> | TGT (C) | CGC (R) | -> | CAC (H) |
| CGC (R) | -> | TGC (C) | CGA (R) | -> | CAA (Q) |
| CGC (R) | -> | CGT (R) | CGG (R) | -> | CAG (Q) |
| CGA (R) | -> | TGA (*) | CGG (R) | -> | CGA (R) |
| CGG (R) | -> | TGG (W) | AGA (R) | -> | AAA (K) |
| TCT (S) | -> | TTT (F) | AGG (R) | -> | AAG (K) |
| TCC (S) | -> | TTC (F) | AGG (R) | -> | AGA (R) |
| TCC (S) | -> | TCT (S) | TCG (S) | -> | TCA (S) |
| TCA (S) | -> | TTA (L) | AGT (S) | -> | AAT (N) |
| TCG (S) | -> | TTG (L) | AGC (S) | -> | AAC (N) |
| AGC (S) | -> | AGT (S) | ACG (T) | -> | ACA (T) |
| ACT (T) | -> | ATT (I) | GTT (V) | -> | ATT (I) |
| ACC (T) | -> | ATC (I) | GTC (V) | -> | ATC (I) |
| ACC (T) | -> | ACT (T) | GTA (V) | -> | ATA (I) |
| ACA (T) | -> | ATA (I) | GTG (V) | -> | ATG (M) |
| ACG (T) | -> | ATG (M) | GTG (V) | -> | GTA (V) |
| GTC (V) | -> | GTT (V) | TGG (W) | -> | TAG (*) |
| TAC (Y) | -> | TAT (Y) | TGG (W) | -> | TGA (*) |

In some embodiments, the activity control component comprises a nucleic acid sequence encoding the amino acid sequence of a Base Editor selected from the group consisting of Cas9 ABE7.10 (SEQ ID NO: 23), Cas9 ABE8.17m (SEQ ID NO: 24), Cas13 ABE REPAIRv1 (SEQ ID NO: 25), and Cas13 ABE REPAIRv2 (SEQ ID NO: 26). In some embodiments, the Base Editor is encoded by a polypeptide comprising at least 80% (e.g., at least 80%, at least 85%, at least 90%, at least 95%, or at least 99%) identity any one of SEQ ID NO: 23-26, wherein the Base Editor is still capable of editing RNA or DNA. In some embodiments, the Base Editor is encoded by a polypeptide comprising the amino acid sequence of any one of SEQ ID NO: 23-26. In some embodiments, the Base Editor is encoded by a polypeptide consisting of the amino acid sequence of any one of SEQ ID NO: 23-26.

In some embodiments, the activity control component comprises a nucleic acid sequence encoding the amino acid sequence of a Base Editor selected from the group consisting of Cas9 AncBE4max (SEQ ID NO: 44), and Cas9 hyBE4max (SEQ ID NO: 45). In some embodiments, the Base Editor is encoded by a polypeptide comprising at least 80% (e.g., at least 80%, at least 85%, at least 90%, at least 95%, or at least 99%) identity any one of SEQ ID NO: 44-45, wherein the Base Editor is still capable of editing RNA or DNA. In some embodiments, the Base Editor is encoded by a polypeptide comprising the amino acid sequence of any one of SEQ ID NO: 44-45. In some embodiments, the Base Editor is encoded by a polypeptide consisting of the amino acid sequence of any one of SEQ ID NO: 44-45.

In some embodiments, the activity control component comprises a nucleic acid sequence encoding a Base Editor (e.g. Cas9 ABE7.10, Cas9 ABE8.17m, Cas13 ABE REPAIRv1, Cas13 ABE REPAIRv2, AncBE4max, or hyBE4max) that is operably linked to a promoter (as described herein). In some embodiments, the promoter is a constitutively active promoter. In some embodiments, the promoter is a chemically inducible promoter. In some embodiments, the Base Editor is operably linked to a chemically inducible promoter selected from the group consisting of pTRE3G (SEQ ID NO: 2) or pTREtight (SEQ ID NO: 1). In some embodiments, the Base Editor is operably linked to a chemically inducible promoter containing at least one of VanR (SEQ ID NO: 27), TtgR (SEQ ID NO: 28), PhlF (SEQ ID NO: 30), or CymR (SEQ ID NOs: 31-32), or the Gal4 UAS (SEQ ID NO: 29) operator sequences.

### B. Base Editor Single Guide RNAs

In some embodiments, the activity control component comprises one or more single guide RNAs. As described herein, the term “single guide RNA” or “sgRNA” refer to RNA sequences capable of binding to and directing a Base Editor to a target DNA or RNA sequence (e.g. DNA or RNA encoding DA-Rep52). Single guide RNAs comprise a nucleic acid sequence referred to as a spacer or protospacer. In some embodiments, the spacer or protospacer is about 15 to 50 base pairs in length and is sufficiently complementary to the target sequence (e.g. DNA or RNA of DA-Rep52) to direct the Base Editor to the target sequence. In some embodiments, the spacer or protospacer is complementary to a target sequence that is adjacent to a protospacer adjacent motif (PAM). In some embodiments, one or more sgRNAs are sufficiently complementary to the mutation(s) within the nucleic acid sequence encoding a gene required for AAV production to direct a Base Editor to the mutation(s) for base editing of the mutation(s) to revert the mutated sequence to the wild-type sequence. In some embodiments, one or more sgRNAs are sufficiently complementary to the mutated start codon(s) within the nucleic acid sequence encoding a gene required for AAV production to direct a Base Editor to the mutated start codon(s) for base editing of the mutated start codon(s). In some embodiments, one or more sgRNAs are sufficiently complementary to the non-synonymous codon(s) within the nucleic acid sequence encoding a gene required for AAV production to direct a Base Editor to the non-synonymous codon(s) for base editing of the non-synonymous codon(s). In some embodiments, one or more sgRNAs are sufficiently complementary to the premature stop codon(s) within the nucleic acid sequence encoding a gene required for AAV production to direct a Base Editor to the premature stop codon(s) for base editing of the premature stop codon(s). In some embodiments, the DNA nucleic acid sequence encoding any sgRNA described herein is operably linked to a promoter (constitutive or inducible, as described herein). In some embodiments, the DNA nucleic acid sequence encoding any sgRNA described herein is operably linked to a U6 promoter.

In some embodiments, the AAV production component comprises a nucleic acid sequence encoding DA-Rep52 and the activity control component comprises a nucleic acid sequence encoding for one or more sgRNAs that are sufficiently complementary to the mutation(s) within the nucleic acid sequence encoding DA-Rep52 to direct a Base Editor to the mutation(s) for base editing of the mutation(s) to revert the mutated sequence to the wild-type sequence as described above (e.g., reverting a mutated start codon to a start codon, a non-synonymous codon to a wild-type codon, and/or a premature stop codon to a wild-type codon).

In some embodiments, the AAV production component comprises a nucleic acid sequence encoding DA-Rep52 and the activity control component comprises a nucleic acid sequence encoding for one or more sgRNAs that are sufficiently complementary to the mutated start codon within the nucleic acid sequence encoding DA-Rep52 to direct a Base Editor to the mutated start codon for base editing of the mutated start codon to a start codon as described above. For example, in some embodiments, the nucleic acid sequence encoding DA-Rep52 comprises a mutated start codon of the sequence ATA in the sense strand (DNA or RNA), and the Base Editor (comprising an ABE) mutates the ATA to ATG. In some embodiments, the nucleic acid sequence encoding for DA-Rep52 comprises a mutated start codon of the sequence ACG in the sense strand (DNA or RNA), and the Base Editor (comprising a CBE) mutates the ACG to ATG. In some embodiments, the nucleic acid sequence encoding for DA-Rep52 comprises a mutated start codon of the sequence GTG in the sense strand (DNA), and the Base Editor (comprising a CBE) mutates the CAC (5′ to 3′) of the antisense strand to TAC (5′ to 3′). In some embodiments, the nucleic acid sequence encoding for DA-Rep52 comprises a mutated start codon of the sequence GCG in the sense strand (DNA), and the Base Editor (comprising a CBE) mutates both the sense and antisense strand to mutate the GCG to ATG (5′ to 3′).

In some embodiments, the AAV production component comprises a nucleic acid sequence encoding DA-Rep52 and the activity control component comprises a nucleic acid sequence encoding for one or more sgRNAs that are sufficiently complementary to the non-synonymous codon(s) within the nucleic acid sequence encoding DA-Rep52 to direct a Base Editor to the non-synonymous codon(s) for base editing of the non-synonymous codon(s) to wild-type codon(s).

In some embodiments, the AAV production component comprises a nucleic acid sequence encoding DA-Rep52 and the activity control component comprises a nucleic acid sequence encoding for one or more sgRNAs that are sufficiently complementary to the premature stop codon within the nucleic acid sequence encoding DA-Rep52 to direct a Base Editor to the premature stop codon for base editing of the premature stop codon to a wild-type codon as described above. For example, in some embodiments, the nucleic acid sequence encoding DA-Rep52 comprises a premature stop codon of the sequence TAG in the sense strand (DNA or RNA), and the Base Editor (comprising an ABE) mutates the TAG to TGG. In some embodiments, the nucleic acid sequence encoding for DA-Rep52 comprises a premature stop codon of the sequence TGA in the sense strand (DNA or RNA), and the Base Editor (comprising an ABE) mutates the TGA to TGG. In some embodiments, the nucleic acid sequence encoding for DA-Rep52 comprises a premature stop codon of the sequence TAA in the sense strand (DNA or RNA), and the Base Editor (comprising an ABE) mutates the TAA to TGG. In some embodiments, the nucleic acid sequence encoding for DA-Rep52 comprises a premature stop codon of the sequence TAA in the sense strand (DNA), and the Base Editor (comprising an ABE) mutates the TTA (5′ to 3′) of the antisense strand to TTG (5′ to 3′). In some embodiments, the nucleic acid sequence encoding for DA-Rep52 comprises a premature stop codon of the sequence TAG in the sense strand (DNA), and the Base Editor (comprising an ABE) mutates the CTA (5′ to 3′) of the antisense strand to CTG (5′ to 3′). In some embodiments, the nucleic acid sequence encoding for DA-Rep52 comprises a premature stop codon of the sequence TGA in the sense strand (DNA), and the Base Editor (comprising an ABE) mutates the TCA (5′ to 3′) of the antisense strand to TCG (5′ to 3′).

In some embodiments, the AAV production component comprises a nucleic acid sequence encoding DA-Rep40 and the activity control component comprises a nucleic acid sequence encoding for one or more sgRNAs that are sufficiently complementary to the mutation(s) within the nucleic acid sequence encoding DA-Rep40 to direct a Base Editor to the mutation(s) for base editing of the mutation(s) to revert the mutated sequence to the wild-type sequence as described above (e.g., reverting a mutated start codon to a start codon, a non-synonymous codon to a wild-type codon, and/or a premature stop codon to a wild-type codon).

In some embodiments, the AAV production component comprises a nucleic acid sequence encoding DA-Rep40 and the activity control component comprises a nucleic acid sequence encoding for one or more sgRNAs that are sufficiently complementary to the mutated start codon within the nucleic acid sequence encoding DA-Rep40 to direct a Base Editor to the mutated start codon for base editing of the mutated start codon to a start codon as described above. For example, in some embodiments, the nucleic acid sequence encoding DA-Rep40 comprises a mutated start codon of the sequence ATA in the sense strand (DNA or RNA), and the Base Editor (comprising an ABE) mutates the ATA to ATG. In some embodiments, the nucleic acid sequence encoding for DA-Rep40 comprises a mutated start codon of the sequence ACG in the sense strand (DNA or RNA), and the Base Editor (comprising a CBE) mutates the ACG to ATG. In some embodiments, the nucleic acid sequence encoding for DA-Rep40 comprises a mutated start codon of the sequence GTG in the sense strand (DNA), and the Base Editor (comprising a CBE) mutates the CAC (5′ to 3′) of the antisense strand to TAC (5′ to 3′). In some embodiments, the nucleic acid sequence encoding for DA-Rep40 comprises a mutated start codon of the sequence GCG in the sense strand (DNA), and the Base Editor (comprising a CBE) mutates both the sense and antisense strand to mutate the GCG to ATG (5′ to 3′).

In some embodiments, the AAV production component comprises a nucleic acid sequence encoding DA-Rep40 and the activity control component comprises a nucleic acid sequence encoding for one or more sgRNAs that are sufficiently complementary to the non-synonymous codon(s) within the nucleic acid sequence encoding DA-Rep40 to direct a Base Editor to the non-synonymous codon(s) for base editing of the non-synonymous codon(s) to wild-type codon(s).

In some embodiments, the AAV production component comprises a nucleic acid sequence encoding DA-Rep40 and the activity control component comprises a nucleic acid sequence encoding for one or more sgRNAs that are sufficiently complementary to the premature stop codon within the nucleic acid sequence encoding DA-Rep40 to direct a Base Editor to the premature stop codon for base editing of the premature stop codon to a wild-type codon as described above. For example, in some embodiments, the nucleic acid sequence encoding DA-Rep40 comprises a premature stop codon of the sequence TAG in the sense strand (DNA or RNA), and the Base Editor (comprising an ABE) mutates the TAG to TGG. In some embodiments, the nucleic acid sequence encoding for DA-Rep40 comprises a premature stop codon of the sequence TGA in the sense strand (DNA or RNA), and the Base Editor (comprising an ABE) mutates the TGA to TGG. In some embodiments, the nucleic acid sequence encoding for DA-Rep40 comprises a premature stop codon of the sequence TAA in the sense strand (DNA or RNA), and the Base Editor (comprising an ABE) mutates the TAA to TGG. In some embodiments, the nucleic acid sequence encoding for DA-Rep40 comprises a premature stop codon of the sequence TAA in the sense strand (DNA), and the Base Editor (comprising an ABE) mutates the TTA (5′ to 3′) of the antisense strand to TTG (5′ to 3′). In some embodiments, the nucleic acid sequence encoding for DA-Rep40 comprises a premature stop codon of the sequence TAG in the sense strand (DNA), and the Base Editor (comprising an ABE) mutates the CTA (5′ to 3′) of the antisense strand to CTG (5′ to 3′). In some embodiments, the nucleic acid sequence encoding for DA-Rep40 comprises a premature stop codon of the sequence TGA in the sense strand (DNA), and the Base Editor (comprising an ABE) mutates the TCA (5′ to 3′) of the antisense strand to TCG (5′ to 3′).

In some embodiments, the AAV production component comprises a nucleic acid sequence encoding DA-Rep78 and the activity control component comprises a nucleic acid sequence encoding for one or more sgRNAs that are sufficiently complementary to the mutation(s) within the nucleic acid sequence encoding DA-Rep78 to direct a Base Editor to the mutation(s) for base editing of the mutation(s) to revert the mutated sequence to the wild-type sequence as described above (e.g., reverting a mutated start codon to a start codon, a non-synonymous codon to a wild-type codon, and/or a premature stop codon to a wild-type codon).

In some embodiments, the AAV production component comprises a nucleic acid sequence encoding DA-Rep78 and the activity control component comprises a nucleic acid sequence encoding for one or more sgRNAs that are sufficiently complementary to the mutated start codon within the nucleic acid sequence encoding DA-Rep78 to direct a Base Editor to the mutated start codon for base editing of the mutated start codon to a start codon as described above. For example, in some embodiments, the nucleic acid sequence encoding DA-Rep78 comprises a mutated start codon of the sequence ATA in the sense strand (DNA or RNA), and the Base Editor (comprising an ABE) mutates the ATA to ATG. In some embodiments, the nucleic acid sequence encoding for DA-Rep78 comprises a mutated start codon of the sequence ACG in the sense strand (DNA or RNA), and the Base Editor (comprising a CBE) mutates the ACG to ATG. In some embodiments, the nucleic acid sequence encoding for DA-Rep78 comprises a mutated start codon of the sequence GTG in the sense strand (DNA), and the Base Editor (comprising a CBE) mutates the CAC (5′ to 3′) of the antisense strand to TAC (5′ to 3′). In some embodiments, the nucleic acid sequence encoding for DA-Rep78 comprises a mutated start codon of the sequence GCG in the sense strand (DNA), and the Base Editor (comprising a CBE) mutates both the sense and antisense strand to mutate the GCG to ATG (5′ to 3′).

In some embodiments, the AAV production component comprises a nucleic acid sequence encoding DA-Rep78 and the activity control component comprises a nucleic acid sequence encoding for one or more sgRNAs that are sufficiently complementary to the non-synonymous codon(s) within the nucleic acid sequence encoding DA-Rep78 to direct a Base Editor to the non-synonymous codon(s) for base editing of the non-synonymous codon(s) to wild-type codon(s).

In some embodiments, the AAV production component comprises a nucleic acid sequence encoding DA-Rep78 and the activity control component comprises a nucleic acid sequence encoding for one or more sgRNAs that are sufficiently complementary to the premature stop codon within the nucleic acid sequence encoding DA-Rep78 to direct a Base Editor to the premature stop codon for base editing of the premature stop codon to a wild-type codon as described above. For example, in some embodiments, the nucleic acid sequence encoding DA-Rep78 comprises a premature stop codon of the sequence TAG in the sense strand (DNA or RNA), and the Base Editor (comprising an ABE) mutates the TAG to TGG. In some embodiments, the nucleic acid sequence encoding for DA-Rep78 comprises a premature stop codon of the sequence TGA in the sense strand (DNA or RNA), and the Base Editor (comprising an ABE) mutates the TGA to TGG. In some embodiments, the nucleic acid sequence encoding for DA-Rep78 comprises a premature stop codon of the sequence TAA in the sense strand (DNA or RNA), and the Base Editor (comprising an ABE) mutates the TAA to TGG. In some embodiments, the nucleic acid sequence encoding for DA-Rep78 comprises a premature stop codon of the sequence TAA in the sense strand (DNA), and the Base Editor (comprising an ABE) mutates the TTA (5′ to 3′) of the antisense strand to TTG (5′ to 3′). In some embodiments, the nucleic acid sequence encoding for DA-Rep78 comprises a premature stop codon of the sequence TAG in the sense strand (DNA), and the Base Editor (comprising an ABE) mutates the CTA (5′ to 3′) of the antisense strand to CTG (5′ to 3′). In some embodiments, the nucleic acid sequence encoding for DA-Rep78 comprises a premature stop codon of the sequence TGA in the sense strand (DNA), and the Base Editor (comprising an ABE) mutates the TCA (5′ to 3′) of the antisense strand to TCG (5′ to 3′).

In some embodiments, the AAV production component comprises a nucleic acid sequence encoding DA-Rep68 and the activity control component comprises a nucleic acid sequence encoding for one or more sgRNAs that are sufficiently complementary to the mutation(s) within the nucleic acid sequence encoding DA-Rep68 to direct a Base Editor to the mutation(s) for base editing of the mutation(s) to revert the mutated sequence to the wild-type sequence as described above (e.g., reverting a mutated start codon to a start codon, a non-synonymous codon to a wild-type codon, and/or a premature stop codon to a wild-type codon).

In some embodiments, the AAV production component comprises a nucleic acid sequence encoding DA-Rep68 and the activity control component comprises a nucleic acid sequence encoding for one or more sgRNAs that are sufficiently complementary to the mutated start codon within the nucleic acid sequence encoding DA-Rep68 to direct a Base Editor to the mutated start codon for base editing of the mutated start codon to a start codon as described above. For example, in some embodiments, the nucleic acid sequence encoding DA-Rep68 comprises a mutated start codon of the sequence ATA in the sense strand (DNA or RNA), and the Base Editor (comprising an ABE) mutates the ATA to ATG. In some embodiments, the nucleic acid sequence encoding for DA-Rep68 comprises a mutated start codon of the sequence ACG in the sense strand (DNA or RNA), and the Base Editor (comprising a CBE) mutates the ACG to ATG. In some embodiments, the nucleic acid sequence encoding for DA-Rep68 comprises a mutated start codon of the sequence GTG in the sense strand (DNA), and the Base Editor (comprising a CBE) mutates the CAC (5′ to 3′) of the antisense strand to TAC (5′ to 3′). In some embodiments, the nucleic acid sequence encoding for DA-Rep68 comprises a mutated start codon of the sequence GCG in the sense strand (DNA), and the Base Editor (comprising a CBE) mutates both the sense and antisense strand to mutate the GCG to ATG (5′ to 3′).

In some embodiments, the AAV production component comprises a nucleic acid sequence encoding DA-Rep68 and the activity control component comprises a nucleic acid sequence encoding for one or more sgRNAs that are sufficiently complementary to the non-synonymous codon(s) within the nucleic acid sequence encoding DA-Rep68 to direct a Base Editor to the non-synonymous codon(s) for base editing of the non-synonymous codon(s) to wild-type codon(s).

In some embodiments, the AAV production component comprises a nucleic acid sequence encoding DA-Rep68 and the activity control component comprises a nucleic acid sequence encoding for one or more sgRNAs that are sufficiently complementary to the premature stop codon within the nucleic acid sequence encoding DA-Rep68 to direct a Base Editor to the premature stop codon for base editing of the premature stop codon to a wild-type codon as described above. For example, in some embodiments, the nucleic acid sequence encoding DA-Rep68 comprises a premature stop codon of the sequence TAG in the sense strand (DNA or RNA), and the Base Editor (comprising an ABE) mutates the TAG to TGG. In some embodiments, the nucleic acid sequence encoding for DA-Rep68 comprises a premature stop codon of the sequence TGA in the sense strand (DNA or RNA), and the Base Editor (comprising an ABE) mutates the TGA to TGG. In some embodiments, the nucleic acid sequence encoding for DA-Rep68 comprises a premature stop codon of the sequence TAA in the sense strand (DNA or RNA), and the Base Editor (comprising an ABE) mutates the TAA to TGG. In some embodiments, the nucleic acid sequence encoding for DA-Rep68 comprises a premature stop codon of the sequence TAA in the sense strand (DNA), and the Base Editor (comprising an ABE) mutates the TTA (5′ to 3′) of the antisense strand to TTG (5′ to 3′). In some embodiments, the nucleic acid sequence encoding for DA-Rep68 comprises a premature stop codon of the sequence TAG in the sense strand (DNA), and the Base Editor (comprising an ABE) mutates the CTA (5′ to 3′) of the antisense strand to CTG (5′ to 3′). In some embodiments, the nucleic acid sequence encoding for DA-Rep68 comprises a premature stop codon of the sequence TGA in the sense strand (DNA), and the Base Editor (comprising an ABE) mutates the TCA (5′ to 3′) of the antisense strand to TCG (5′ to 3′).

In some embodiments, the AAV production component comprises a nucleic acid sequence encoding DA-E2A and the activity control component comprises a nucleic acid sequence encoding for one or more sgRNAs that are sufficiently complementary to the mutation(s) within the nucleic acid sequence encoding DA-E2A to direct a Base Editor to the mutation(s) for base editing of the mutation(s) to revert the mutated sequence to the wild-type sequence as described above (e.g., reverting a mutated start codon to a start codon, a non-synonymous codon to a wild-type codon, and/or a premature stop codon to a wild-type codon).

In some embodiments, the AAV production component comprises a nucleic acid sequence encoding DA-E2A and the activity control component comprises a nucleic acid sequence encoding for one or more sgRNAs that are sufficiently complementary to the mutated start codon within the nucleic acid sequence encoding DA-E2A to direct a Base Editor to the mutated start codon for base editing of the mutated start codon to a start codon as described above. For example, in some embodiments, the nucleic acid sequence encoding DA-E2A comprises a mutated start codon of the sequence ATA in the sense strand (DNA or RNA), and the Base Editor (comprising an ABE) mutates the ATA to ATG. In some embodiments, the nucleic acid sequence encoding for DA-E2A comprises a mutated start codon of the sequence ACG in the sense strand (DNA or RNA), and the Base Editor (comprising a CBE) mutates the ACG to ATG. In some embodiments, the nucleic acid sequence encoding for DA-E2A comprises a mutated start codon of the sequence GTG in the sense strand (DNA), and the Base Editor (comprising a CBE) mutates the CAC (5′ to 3′) of the antisense strand to TAC (5′ to 3′). In some embodiments, the nucleic acid sequence encoding for DA-E2A comprises a mutated start codon of the sequence GCG in the sense strand (DNA), and the Base Editor (comprising a CBE) mutates both the sense and antisense strand to mutate the GCG to ATG (5′ to 3′).

In some embodiments, the AAV production component comprises a nucleic acid sequence encoding DA-E2A and the activity control component comprises a nucleic acid sequence encoding for one or more sgRNAs that are sufficiently complementary to the non-synonymous codon(s) within the nucleic acid sequence encoding DA-E2A to direct a Base Editor to the non-synonymous codon(s) for base editing of the non-synonymous codon(s) to wild-type codon(s).

In some embodiments, the AAV production component comprises a nucleic acid sequence encoding DA-E2A and the activity control component comprises a nucleic acid sequence encoding for one or more sgRNAs that are sufficiently complementary to the premature stop codon within the nucleic acid sequence encoding DA-E2A to direct a Base Editor to the premature stop codon for base editing of the premature stop codon to a wild-type codon as described above. For example, in some embodiments, the nucleic acid sequence encoding DA-E2A comprises a premature stop codon of the sequence TAG in the sense strand (DNA or RNA), and the Base Editor (comprising an ABE) mutates the TAG to TGG. In some embodiments, the nucleic acid sequence encoding for DA-E2A comprises a premature stop codon of the sequence TGA in the sense strand (DNA or RNA), and the Base Editor (comprising an ABE) mutates the TGA to TGG. In some embodiments, the nucleic acid sequence encoding for DA-E2A comprises a premature stop codon of the sequence TAA in the sense strand (DNA or RNA), and the Base Editor (comprising an ABE) mutates the TAA to TGG. In some embodiments, the nucleic acid sequence encoding for DA-E2A comprises a premature stop codon of the sequence TAA in the sense strand (DNA), and the Base Editor (comprising an ABE) mutates the TTA (5′ to 3′) of the antisense strand to TTG (5′ to 3′). In some embodiments, the nucleic acid sequence encoding for DA-E2A comprises a premature stop codon of the sequence TAG in the sense strand (DNA), and the Base Editor (comprising an ABE) mutates the CTA (5′ to 3′) of the antisense strand to CTG (5′ to 3′). In some embodiments, the nucleic acid sequence encoding for DA-E2A comprises a premature stop codon of the sequence TGA in the sense strand (DNA), and the Base Editor (comprising an ABE) mutates the TCA (5′ to 3′) of the antisense strand to TCG (5′ to 3′).

In some embodiments, the AAV production component comprises a nucleic acid sequence encoding DA-E4ORF6 and the activity control component comprises a nucleic acid sequence encoding for one or more sgRNAs that are sufficiently complementary to the mutation(s) within the nucleic acid sequence encoding DA-E4ORF6 to direct a Base Editor to the mutation(s) for base editing of the mutation(s) to revert the mutated sequence to the wild-type sequence as described above (e.g., reverting a mutated start codon to a start codon, a non-synonymous codon to a wild-type codon, and/or a premature stop codon to a wild-type codon).

In some embodiments, the AAV production component comprises a nucleic acid sequence encoding DA-E4ORF6 and the activity control component comprises a nucleic acid sequence encoding for one or more sgRNAs that are sufficiently complementary to the mutated start codon within the nucleic acid sequence encoding DA-E4ORF6 to direct a Base Editor to the mutated start codon for base editing of the mutated start codon to a start codon as described above. For example, in some embodiments, the nucleic acid sequence encoding DA-E4ORF6 comprises a mutated start codon of the sequence ATA in the sense strand (DNA or RNA), and the Base Editor (comprising an ABE) mutates the ATA to ATG. In some embodiments, the nucleic acid sequence encoding for DA-E4ORF6 comprises a mutated start codon of the sequence ACG in the sense strand (DNA or RNA), and the Base Editor (comprising a CBE) mutates the ACG to ATG. In some embodiments, the nucleic acid sequence encoding for DA-E4ORF6 comprises a mutated start codon of the sequence GTG in the sense strand (DNA), and the Base Editor (comprising a CBE) mutates the CAC (5′ to 3′) of the antisense strand to TAC (5′ to 3′). In some embodiments, the nucleic acid sequence encoding for DA-E4ORF6 comprises a mutated start codon of the sequence GCG in the sense strand (DNA), and the Base Editor (comprising a CBE) mutates both the sense and antisense strand to mutate the GCG to ATG (5′ to 3′).

In some embodiments, the AAV production component comprises a nucleic acid sequence encoding DA-E4ORF6 and the activity control component comprises a nucleic acid sequence encoding for one or more sgRNAs that are sufficiently complementary to the non-synonymous codon(s) within the nucleic acid sequence encoding DA-E4ORF6 to direct a Base Editor to the non-synonymous codon(s) for base editing of the non-synonymous codon(s) to wild-type codon(s).

In some embodiments, the AAV production component comprises a nucleic acid sequence encoding DA-E4ORF6 and the activity control component comprises a nucleic acid sequence encoding for one or more sgRNAs that are sufficiently complementary to the premature stop codon within the nucleic acid sequence encoding DA-E4ORF6 to direct a Base Editor to the premature stop codon for base editing of the premature stop codon to a wild-type codon as described above. For example, in some embodiments, the nucleic acid sequence encoding DA-E4ORF6 comprises a premature stop codon of the sequence TAG in the sense strand (DNA or RNA), and the Base Editor (comprising an ABE) mutates the TAG to TGG. In some embodiments, the nucleic acid sequence encoding for DA-E4ORF6 comprises a premature stop codon of the sequence TGA in the sense strand (DNA or RNA), and the Base Editor (comprising an ABE) mutates the TGA to TGG. In some embodiments, the nucleic acid sequence encoding for DA-E4ORF6 comprises a premature stop codon of the sequence TAA in the sense strand (DNA or RNA), and the Base Editor (comprising an ABE) mutates the TAA to TGG. In some embodiments, the nucleic acid sequence encoding for DA-E4ORF6 comprises a premature stop codon of the sequence TAA in the sense strand (DNA), and the Base Editor (comprising an ABE) mutates the TTA (5′ to 3′) of the antisense strand to TTG (5′ to 3′). In some embodiments, the nucleic acid sequence encoding for DA-E4ORF6 comprises a premature stop codon of the sequence TAG in the sense strand (DNA), and the Base Editor (comprising an ABE) mutates the CTA (5′ to 3′) of the antisense strand to CTG (5′ to 3′). In some embodiments, the nucleic acid sequence encoding for DA-E4ORF6 comprises a premature stop codon of the sequence TGA in the sense strand (DNA), and the Base Editor (comprising an ABE) mutates the TCA (5′ to 3′) of the antisense strand to TCG (5′ to 3′).

In some embodiments, the AAV production component comprises a nucleic acid sequence encoding DA-VP1 and the activity control component comprises a nucleic acid sequence encoding for one or more sgRNAs that are sufficiently complementary to the mutation(s) within the nucleic acid sequence encoding DA-VP1 to direct a Base Editor to the mutation(s) for base editing of the mutation(s) to revert the mutated sequence to the wild-type sequence as described above (e.g., reverting a mutated start codon to a start codon, a non-synonymous codon to a wild-type codon, and/or a premature stop codon to a wild-type codon).

In some embodiments, the AAV production component comprises a nucleic acid sequence encoding DA-VP1 and the activity control component comprises a nucleic acid sequence encoding for one or more sgRNAs that are sufficiently complementary to the mutated start codon within the nucleic acid sequence encoding DA-VP1 to direct a Base Editor to the mutated start codon for base editing of the mutated start codon to a start codon as described above. For example, in some embodiments, the nucleic acid sequence encoding DA-VP1 comprises a mutated start codon of the sequence ATA in the sense strand (DNA or RNA), and the Base Editor (comprising an ABE) mutates the ATA to ATG. In some embodiments, the nucleic acid sequence encoding for DA-VP1 comprises a mutated start codon of the sequence ACG in the sense strand (DNA or RNA), and the Base Editor (comprising a CBE) mutates the ACG to ATG. In some embodiments, the nucleic acid sequence encoding for DA-VP1 comprises a mutated start codon of the sequence GTG in the sense strand (DNA), and the Base Editor (comprising a CBE) mutates the CAC (5′ to 3′) of the antisense strand to TAC (5′ to 3′). In some embodiments, the nucleic acid sequence encoding for DA-VP1 comprises a mutated start codon of the sequence GCG in the sense strand (DNA), and the Base Editor (comprising a CBE) mutates both the sense and antisense strand to mutate the GCG to ATG (5′ to 3′).

In some embodiments, the AAV production component comprises a nucleic acid sequence encoding DA-VP1 and the activity control component comprises a nucleic acid sequence encoding for one or more sgRNAs that are sufficiently complementary to the non-synonymous codon(s) within the nucleic acid sequence encoding DA-VP1 to direct a Base Editor to the non-synonymous codon(s) for base editing of the non-synonymous codon(s) to wild-type codon(s).

In some embodiments, the AAV production component comprises a nucleic acid sequence encoding DA-VP1 and the activity control component comprises a nucleic acid sequence encoding for one or more sgRNAs that are sufficiently complementary to the premature stop codon within the nucleic acid sequence encoding DA-VP1 to direct a Base Editor to the premature stop codon for base editing of the premature stop codon to a wild-type codon as described above. For example, in some embodiments, the nucleic acid sequence encoding DA-VP1 comprises a premature stop codon of the sequence TAG in the sense strand (DNA or RNA), and the Base Editor (comprising an ABE) mutates the TAG to TGG. In some embodiments, the nucleic acid sequence encoding for DA-VP1 comprises a premature stop codon of the sequence TGA in the sense strand (DNA or RNA), and the Base Editor (comprising an ABE) mutates the TGA to TGG. In some embodiments, the nucleic acid sequence encoding for DA-VP1 comprises a premature stop codon of the sequence TAA in the sense strand (DNA or RNA), and the Base Editor (comprising an ABE) mutates the TAA to TGG. In some embodiments, the nucleic acid sequence encoding for DA-VP1 comprises a premature stop codon of the sequence TAA in the sense strand (DNA), and the Base Editor (comprising an ABE) mutates the TTA (5′ to 3′) of the antisense strand to TTG (5′ to 3′). In some embodiments, the nucleic acid sequence encoding for DA-VP1 comprises a premature stop codon of the sequence TAG in the sense strand (DNA), and the Base Editor (comprising an ABE) mutates the CTA (5′ to 3′) of the antisense strand to CTG (5′ to 3′). In some embodiments, the nucleic acid sequence encoding for DA-VP1 comprises a premature stop codon of the sequence TGA in the sense strand (DNA), and the Base Editor (comprising an ABE) mutates the TCA (5′ to 3′) of the antisense strand to TCG (5′ to 3′).

In some embodiments, the AAV production component comprises a nucleic acid sequence encoding DA-VP2 and the activity control component comprises a nucleic acid sequence encoding for one or more sgRNAs that are sufficiently complementary to the mutation(s) within the nucleic acid sequence encoding DA-VP2 to direct a Base Editor to the mutation(s) for base editing of the mutation(s) to revert the mutated sequence to the wild-type sequence as described above (e.g., reverting a mutated start codon to a start codon, a non-synonymous codon to a wild-type codon, and/or a premature stop codon to a wild-type codon).

In some embodiments, the AAV production component comprises a nucleic acid sequence encoding DA-VP2 and the activity control component comprises a nucleic acid sequence encoding for one or more sgRNAs that are sufficiently complementary to the mutated start codon within the nucleic acid sequence encoding DA-VP2 to direct a Base Editor to the mutated start codon for base editing of the mutated start codon to a start codon as described above. For example, in some embodiments, the nucleic acid sequence encoding DA-VP2 comprises a mutated start codon of the sequence ACA in the sense strand (DNA or RNA), and the Base Editor (comprising an ABE) mutates the ACA to the ACG start codon. In some embodiments, the nucleic acid sequence encoding for DA-VP2 comprises a mutated start codon of the sequence GCG in the sense strand (DNA), and the Base Editor (comprising a CBE) mutates the CGC (5′ to 3′) of the antisense strand to CGT (5′ to 3′).

In some embodiments, the AAV production component comprises a nucleic acid sequence encoding DA-VP2 and the activity control component comprises a nucleic acid sequence encoding for one or more sgRNAs that are sufficiently complementary to the non-synonymous codon(s) within the nucleic acid sequence encoding DA-VP2 to direct a Base Editor to the non-synonymous codon(s) for base editing of the non-synonymous codon(s) to wild-type codon(s).

In some embodiments, the AAV production component comprises a nucleic acid sequence encoding DA-VP2 and the activity control component comprises a nucleic acid sequence encoding for one or more sgRNAs that are sufficiently complementary to the premature stop codon within the nucleic acid sequence encoding DA-VP2 to direct a Base Editor to the premature stop codon for base editing of the premature stop codon to a wild-type codon as described above. For example, in some embodiments, the nucleic acid sequence encoding DA-VP2 comprises a premature stop codon of the sequence TAG in the sense strand (DNA or RNA), and the Base Editor (comprising an ABE) mutates the TAG to TGG. In some embodiments, the nucleic acid sequence encoding for DA-VP2 comprises a premature stop codon of the sequence TGA in the sense strand (DNA or RNA), and the Base Editor (comprising an ABE) mutates the TGA to TGG. In some embodiments, the nucleic acid sequence encoding for DA-VP2 comprises a premature stop codon of the sequence TAA in the sense strand (DNA or RNA), and the Base Editor (comprising an ABE) mutates the TAA to TGG. In some embodiments, the nucleic acid sequence encoding for DA-VP2 comprises a premature stop codon of the sequence TAA in the sense strand (DNA), and the Base Editor (comprising an ABE) mutates the TTA (5′ to 3′) of the antisense strand to TTG (5′ to 3′). In some embodiments, the nucleic acid sequence encoding for DA-VP2 comprises a premature stop codon of the sequence TAG in the sense strand (DNA), and the Base Editor (comprising an ABE) mutates the CTA (5′ to 3′) of the antisense strand to CTG (5′ to 3′). In some embodiments, the nucleic acid sequence encoding for DA-VP2 comprises a premature stop codon of the sequence TGA in the sense strand (DNA), and the Base Editor (comprising an ABE) mutates the TCA (5′ to 3′) of the antisense strand to TCG (5′ to 3′).

In some embodiments, the AAV production component comprises a nucleic acid sequence encoding DA-VP3 and the activity control component comprises a nucleic acid sequence encoding for one or more sgRNAs that are sufficiently complementary to the mutation(s) within the nucleic acid sequence encoding DA-VP3 to direct a Base Editor to the mutation(s) for base editing of the mutation(s) to revert the mutated sequence to the wild-type sequence as described above (e.g., reverting a mutated start codon to a start codon, a non-synonymous codon to a wild-type codon, and/or a premature stop codon to a wild-type codon).

In some embodiments, the AAV production component comprises a nucleic acid sequence encoding DA-VP3 and the activity control component comprises a nucleic acid sequence encoding for one or more sgRNAs that are sufficiently complementary to the mutated start codon within the nucleic acid sequence encoding DA-VP3 to direct a Base Editor to the mutated start codon for base editing of the mutated start codon to a start codon as described above. For example, in some embodiments, the nucleic acid sequence encoding DA-VP3 comprises a mutated start codon of the sequence ATA in the sense strand (DNA or RNA), and the Base Editor (comprising an ABE) mutates the ATA to ATG. In some embodiments, the nucleic acid sequence encoding for DA-VP3 comprises a mutated start codon of the sequence ACG in the sense strand (DNA or RNA), and the Base Editor (comprising a CBE) mutates the ACG to ATG. In some embodiments, the nucleic acid sequence encoding for DA-VP3 comprises a mutated start codon of the sequence GTG in the sense strand (DNA), and the Base Editor (comprising a CBE) mutates the CAC (5′ to 3′) of the antisense strand to TAC (5′ to 3′). In some embodiments, the nucleic acid sequence encoding for DA-VP3 comprises a mutated start codon of the sequence GCG in the sense strand (DNA), and the Base Editor (comprising a CBE) mutates both the sense and antisense strand to mutate the GCG to ATG (5′ to 3′).

In some embodiments, the AAV production component comprises a nucleic acid sequence encoding DA-VP3 and the activity control component comprises a nucleic acid sequence encoding for one or more sgRNAs that are sufficiently complementary to the non-synonymous codon(s) within the nucleic acid sequence encoding DA-VP3 to direct a Base Editor to the non-synonymous codon(s) for base editing of the non-synonymous codon(s) to wild-type codon(s).

In some embodiments, the AAV production component comprises a nucleic acid sequence encoding DA-VP3 and the activity control component comprises a nucleic acid sequence encoding for one or more sgRNAs that are sufficiently complementary to the premature stop codon within the nucleic acid sequence encoding DA-VP3 to direct a Base Editor to the premature stop codon for base editing of the premature stop codon to a wild-type codon as described above. For example, in some embodiments, the nucleic acid sequence encoding DA-VP3 comprises a premature stop codon of the sequence TAG in the sense strand (DNA or RNA), and the Base Editor (comprising an ABE) mutates the TAG to TGG. In some embodiments, the nucleic acid sequence encoding for DA-VP3 comprises a premature stop codon of the sequence TGA in the sense strand (DNA or RNA), and the Base Editor (comprising an ABE) mutates the TGA to TGG. In some embodiments, the nucleic acid sequence encoding for DA-VP3 comprises a premature stop codon of the sequence TAA in the sense strand (DNA or RNA), and the Base Editor (comprising an ABE) mutates the TAA to TGG. In some embodiments, the nucleic acid sequence encoding for DA-VP3 comprises a premature stop codon of the sequence TAA in the sense strand (DNA), and the Base Editor (comprising an ABE) mutates the TTA (5′ to 3′) of the antisense strand to TTG (5′ to 3′). In some embodiments, the nucleic acid sequence encoding for DA-VP3 comprises a premature stop codon of the sequence TAG in the sense strand (DNA), and the Base Editor (comprising an ABE) mutates the CTA (5′ to 3′) of the antisense strand to CTG (5′ to 3′). In some embodiments, the nucleic acid sequence encoding for DA-VP3 comprises a premature stop codon of the sequence TGA in the sense strand (DNA), and the Base Editor (comprising an ABE) mutates the TCA (5′ to 3′) of the antisense strand to TCG (5′ to 3′).

In some embodiments, the AAV production component comprises a nucleic acid sequence encoding DA-AAP and the activity control component comprises a nucleic acid sequence encoding for one or more sgRNAs that are sufficiently complementary to the mutation(s) within the nucleic acid sequence encoding DA-AAP to direct a Base Editor to the mutation(s) for base editing of the mutation(s) to revert the mutated sequence to the wild-type sequence as described above (e.g., reverting a mutated start codon to a start codon, a non-synonymous codon to a wild-type codon, and/or a premature stop codon to a wild-type codon).

In some embodiments, the AAV production component comprises a nucleic acid sequence encoding DA-AAP and the activity control component comprises a nucleic acid sequence encoding for one or more sgRNAs that are sufficiently complementary to the mutated start codon within the nucleic acid sequence encoding DA-AAP to direct a Base Editor to the mutated start codon for base editing of the mutated start codon to a start codon as described above. For example, in some embodiments, the nucleic acid sequence encoding DA-AAP comprises a mutated start codon of the sequence CTA in the sense strand (DNA or RNA), and the Base Editor (comprising an ABE) mutates the CTA to CTG. In some embodiments, the nucleic acid sequence encoding for DA-AAP comprises a mutated start codon of the sequence TTG in the sense strand (DNA or RNA), and the Base Editor (comprising a CBE) mutates the TTG to CTG. In some embodiments, the nucleic acid sequence encoding for DA-AAP comprises a mutated start codon of the sequence CCG in the sense strand (DNA), and the Base Editor (comprising a CBE) mutates the CGG (5′ to 3′) of the antisense strand to CAG (5′ to 3′).

In some embodiments, the AAV production component comprises a nucleic acid sequence encoding DA-AAP and the activity control component comprises a nucleic acid sequence encoding for one or more sgRNAs that are sufficiently complementary to the non-synonymous codon(s) within the nucleic acid sequence encoding DA-AAP to direct a Base Editor to the non-synonymous codon(s) for base editing of the non-synonymous codon(s) to wild-type codon(s).

In some embodiments, the AAV production component comprises a nucleic acid sequence encoding DA-AAP and the activity control component comprises a nucleic acid sequence encoding for one or more sgRNAs that are sufficiently complementary to the premature stop codon within the nucleic acid sequence encoding DA-AAP to direct a Base Editor to the premature stop codon for base editing of the premature stop codon to a wild-type codon as described above. For example, in some embodiments, the nucleic acid sequence encoding DA-AAP comprises a premature stop codon of the sequence TAG in the sense strand (DNA or RNA), and the Base Editor (comprising an ABE) mutates the TAG to TGG. In some embodiments, the nucleic acid sequence encoding for DA-AAP comprises a premature stop codon of the sequence TGA in the sense strand (DNA or RNA), and the Base Editor (comprising an ABE) mutates the TGA to TGG. In some embodiments, the nucleic acid sequence encoding for DA-AAP comprises a premature stop codon of the sequence TAA in the sense strand (DNA or RNA), and the Base Editor (comprising an ABE) mutates the TAA to TGG. In some embodiments, the nucleic acid sequence encoding for DA-AAP comprises a premature stop codon of the sequence TAA in the sense strand (DNA), and the Base Editor (comprising an ABE) mutates the TTA (5′ to 3′) of the antisense strand to TTG (5′ to 3′). In some embodiments, the nucleic acid sequence encoding for DA-AAP comprises a premature stop codon of the sequence TAG in the sense strand (DNA), and the Base Editor (comprising an ABE) mutates the CTA (5′ to 3′) of the antisense strand to CTG (5′ to 3′). In some embodiments, the nucleic acid sequence encoding for DA-AAP comprises a premature stop codon of the sequence TGA in the sense strand (DNA), and the Base Editor (comprising an ABE) mutates the TCA (5′ to 3′) of the antisense strand to TCG (5′ to 3′).

In some embodiments, the AAV production component comprises a nucleic acid sequence encoding DA-MAAP and the activity control component comprises a nucleic acid sequence encoding for one or more sgRNAs that are sufficiently complementary to the mutation(s) within the nucleic acid sequence encoding DA-MAAP to direct a Base Editor to the mutation(s) for base editing of the mutation(s) to revert the mutated sequence to the wild-type sequence as described above (e.g., reverting a mutated start codon to a start codon, and/or a non-synonymous codon to a wild-type codon, and/or a premature stop codon to a wild-type codon).

In some embodiments, the AAV production component comprises a nucleic acid sequence encoding DA-MAAP and the activity control component comprises a nucleic acid sequence encoding for one or more sgRNAs that are sufficiently complementary to the mutated start codon within the nucleic acid sequence encoding DA-MAAP to direct a Base Editor to the mutated start codon for base editing of the mutated start codon to a start codon as described above. For example, in some embodiments, the nucleic acid sequence encoding DA-MAAP comprises a mutated start codon of the sequence CTA in the sense strand (DNA or RNA), and the Base Editor (comprising an ABE) mutates the CTA to CTG. In some embodiments, the nucleic acid sequence encoding for DA-MAAP comprises a mutated start codon of the sequence TTG in the sense strand (DNA or RNA), and the Base Editor (comprising a CBE) mutates the TTG to CTG. In some embodiments, the nucleic acid sequence encoding for DA-MAAP comprises a mutated start codon of the sequence CCG in the sense strand (DNA), and the Base Editor (comprising a CBE) mutates the CGG (5′ to 3′) of the antisense strand to CAG (5′ to 3′).

In some embodiments, the AAV production component comprises a nucleic acid sequence encoding DA-MAAP and the activity control component comprises a nucleic acid sequence encoding for one or more sgRNAs that are sufficiently complementary to the non-synonymous codon(s) within the nucleic acid sequence encoding DA-MAAP to direct a Base Editor to the non-synonymous codon(s) for base editing of the non-synonymous codon(s) to wild-type codon(s).

In some embodiments, the AAV production component comprises a nucleic acid sequence encoding DA-MAAP and the activity control component comprises a nucleic acid sequence encoding for one or more sgRNAs that are sufficiently complementary to the premature stop codon within the nucleic acid sequence encoding DA-MAAP to direct a Base Editor to the premature stop codon for base editing of the premature stop codon to a wild-type codon as described above. For example, in some embodiments, the nucleic acid sequence encoding DA-MAAP comprises a premature stop codon of the sequence TAG in the sense strand (DNA or RNA), and the Base Editor (comprising an ABE) mutates the TAG to TGG. In some embodiments, the nucleic acid sequence encoding for DA-MAAP comprises a premature stop codon of the sequence TGA in the sense strand (DNA or RNA), and the Base Editor (comprising an ABE) mutates the TGA to TGG. In some embodiments, the nucleic acid sequence encoding for DA-MAAP comprises a premature stop codon of the sequence TAA in the sense strand (DNA or RNA), and the Base Editor (comprising an ABE) mutates the TAA to TGG. In some embodiments, the nucleic acid sequence encoding for DA-MAAP comprises a premature stop codon of the sequence TAA in the sense strand (DNA), and the Base Editor (comprising an ABE) mutates the TTA (5′ to 3′) of the antisense strand to TTG (5′ to 3′). In some embodiments, the nucleic acid sequence encoding for DA-MAAP comprises a premature stop codon of the sequence TAG in the sense strand (DNA), and the Base Editor (comprising an ABE) mutates the CTA (5′ to 3′) of the antisense strand to CTG (5′ to 3′). In some embodiments, the nucleic acid sequence encoding for DA-MAAP comprises a premature stop codon of the sequence TGA in the sense strand (DNA), and the Base Editor (comprising an ABE) mutates the TCA (5′ to 3′) of the antisense strand to TCG (5′ to 3′).

In some embodiments, the AAV production component comprises a nucleic acid sequence encoding DA-L4 100K and the activity control component comprises a nucleic acid sequence encoding for one or more sgRNAs that are sufficiently complementary to the mutation(s) within the nucleic acid sequence encoding DA-L4 100K to direct a Base Editor to the mutation(s) for base editing of the mutation(s) to revert the mutated sequence to the wild-type sequence as described above (e.g., reverting a mutated start codon to a start codon, a non-synonymous codon to a wild-type codon, and/or a premature stop codon to a wild-type codon).

In some embodiments, the AAV production component comprises a nucleic acid sequence encoding DA-L4 100K and the activity control component comprises a nucleic acid sequence encoding for one or more sgRNAs that are sufficiently complementary to the mutated start codon within the nucleic acid sequence encoding DA-L4 100K to direct a Base Editor to the mutated start codon for base editing of the mutated start codon to a start codon as described above. For example, in some embodiments, the nucleic acid sequence encoding DA-L4 100K comprises a mutated start codon of the sequence ATA in the sense strand (DNA or RNA), and the Base Editor (comprising an ABE) mutates the ATA to ATG. In some embodiments, the nucleic acid sequence encoding for DA-L4 100K comprises a mutated start codon of the sequence ACG in the sense strand (DNA or RNA), and the Base Editor (comprising a CBE) mutates the ACG to ATG. In some embodiments, the nucleic acid sequence encoding for DA-L4 100K comprises a mutated start codon of the sequence GTG in the sense strand (DNA), and the Base Editor (comprising a CBE) mutates the CAC (5′ to 3′) of the antisense strand to TAC (5′ to 3′). In some embodiments, the nucleic acid sequence encoding for DA-L4 100K comprises a mutated start codon of the sequence GCG in the sense strand (DNA), and the Base Editor (comprising a CBE) mutates both the sense and antisense strand to mutate the GCG to ATG (5′ to 3′).

In some embodiments, the AAV production component comprises a nucleic acid sequence encoding DA-L4 100K and the activity control component comprises a nucleic acid sequence encoding for one or more sgRNAs that are sufficiently complementary to the non-synonymous codon(s) within the nucleic acid sequence encoding DA-L4 100K to direct a Base Editor to the non-synonymous codon(s) for base editing of the non-synonymous codon(s) to wild-type codon(s) as described above.

In some embodiments, the AAV production component comprises a nucleic acid sequence encoding DA-L4 100K and the activity control component comprises a nucleic acid sequence encoding for one or more sgRNAs that are sufficiently complementary to the premature stop codon within the nucleic acid sequence encoding DA-L4 100K to direct a Base Editor to the premature stop codon for base editing of the premature stop codon to a wild-type codon as described above. For example, in some embodiments, the nucleic acid sequence encoding DA-L4 100K comprises a premature stop codon of the sequence TAG in the sense strand (DNA or RNA), and the Base Editor (comprising an ABE) mutates the TAG to TGG. In some embodiments, the nucleic acid sequence encoding for DA-L4 100K comprises a premature stop codon of the sequence TGA in the sense strand (DNA or RNA), and the Base Editor (comprising an ABE) mutates the TGA to TGG. In some embodiments, the nucleic acid sequence encoding for DA-L4 100K comprises a premature stop codon of the sequence TAA in the sense strand (DNA or RNA), and the Base Editor (comprising an ABE) mutates the TAA to TGG. In some embodiments, the nucleic acid sequence encoding for DA-L4 100K comprises a premature stop codon of the sequence TAA in the sense strand (DNA), and the Base Editor (comprising an ABE) mutates the TTA (5′ to 3′) of the antisense strand to TTG (5′ to 3′). In some embodiments, the nucleic acid sequence encoding for DA-L4 100K comprises a premature stop codon of the sequence TAG in the sense strand (DNA), and the Base Editor (comprising an ABE) mutates the CTA (5′ to 3′) of the antisense strand to CTG (5′ to 3′). In some embodiments, the nucleic acid sequence encoding for DA-L4 100K comprises a premature stop codon of the sequence TGA in the sense strand (DNA), and the Base Editor (comprising an ABE) mutates the TCA (5′ to 3′) of the antisense strand to TCG (5′ to 3′).

### III. Engineered Cells

In some aspects, the disclosure relates to engineered cells for AAV production. An engineered cell may comprise any part (and any combination of parts) of the AAV production systems described herein.

For example, an engineered cell may comprise at least a portion of the AAV production component. For example, and as described above, an AAV production component may comprise multiple polynucleic acids. In such embodiments, an engineered cell comprises one or more of said multiple polynucleic acids—each of which may be located extra-chromosomally or stably integrated into the genome of the engineered cell. In some embodiments, an engineered cell comprises the entire AAV production component.

Alternatively, or in addition, an engineered cell may comprise the activity control component of the AAV production system.

In some embodiments, an AAV production system comprises: (a) an engineered cell comprising an AAV production component comprising one or more heterologous polynucleic acids that collectively encode the genes required for AAV production, wherein at least one gene comprises a mutation; (b) an activity control component capable of inducing production and/or correcting the mutation of the at least one gene comprising a mutation.

In some embodiments, the engineered cells are derived from known or existing cell lines. In some embodiments, the engineered cells are derived from the group consisting of HEK293 cells, HeLa cells, BHK cells, and Sf9 cells. In some embodiments, the engineered cells comprise nucleic acid sequences encoding genes required for AAV production and systems for regulating expression of said genes, as described herein. In some embodiments, the engineered cell comprises genomic sites for stable integration of one or more nucleic acid molecules (e.g. 1, 2, 3, 4, 5, or 6 nucleic acid molecules). These genomics sites for stable integration of nucleic acid molecules are well known to those of ordinary skill in the art. Exemplary sites for stable integration include but are not limited to AAVS1, ROSA26, CCR5, H11, and LiPS-A3S. In some embodiments, the stably integrated nucleic acid molecule is randomly integrated into the Engineered cell genome.

An engineered cell described herein may further comprise a landing pad. As used herein, the term “landing pad” refers to a heterologous polynucleic acid sequence that facilitates the targeted insertion of a “payload” sequence into a specific locus (or multiple loci) of the cell's genome. Accordingly, the landing pad is integrated into the genome of the cell. A fixed integration site is desirable to reduce the variability between experiments that may be caused by positional epigenetic effects or proximal regulatory elements. The ability to control payload copy number is also desirable to modulate expression levels of the payload without changing any genetic components.

In some embodiments, the landing pad is located at a safe harbor site in the genome of the engineered cell. As used herein, the term “safe harbor site” refers to a location in the genome where genes or genetic elements can be introduced without disrupting the expression or regulation of adjacent genes and/or adjacent genomic elements do not disrupt expression or regulation of the introduced genes or genetic elements. Examples of safe harbor sites are known to those having skill in the art and include, but are not limited to, AAVS1, ROSA26, COSMIC, H11, CCR5, and LiPS-A3S. See e.g., Gaidukov et al., Nucleic Acids Res. 2018 May 4; 46(8): 4072-4086; U.S. Pat. Nos. 8,980,579 B2; 10,017,786 B2; 9,932,607 B2; Pub. No.: US 2013/280222 A; Pub. No.: WO 2017/180669 A1—the entireties of which are incorporated by reference herein, particularly for the disclosure relating to safe harbor sites. In some embodiments, the safe harbor site is a known site. In other embodiments, the safe harbor site is a previously undisclosed site. See “Methods of Identifying High-Expressing Genomic Loci and Uses Thereof” herein. In some embodiments, an engineered cell described herein comprises a landing pad that is integrated at a safe harbor locus selected from the group consisting of AAVS1, ROSA26, COSMIC, H11, CCR5, and LiPS-A3S.

In some embodiments, the engineered cell is derived from a HEK293 cell. In some embodiments, the engineered HEK293 cell comprises a landing pad that is integrated at a safe harbor locus selected from the group consisting of AAVS1, ROSA26, CCR5, and LiPS-A3S.

In some embodiments, the engineered cell is derived from a CHO cell. In some embodiments, the engineered CHO cell comprises a landing pad that is integrated at a safe harbor locus selected from the group consisting of ROSA26, COSMIC, and H11.

Each of the landing pads described herein comprises at least one recombination site. Recombination sites for various integrases have been identified previously. For example, a landing pad may comprise recombination sites corresponding to a Bxb1 integrase, lambda-integrase, Cre recombinase, Flp recombinase, gamma-delta resolvase, Tn3 resolvase, qC31 integrase, or R4 integrase. Exemplary recombination site sequences are known in the art (e.g., attP, attB, attR, attL, Lox, and Frt).

The landing pads described herein may comprise one or more expression cassettes.

In some embodiments, the payload sequence comprises a nucleic acid molecule encoding a first inverted terminal repeat (ITR), a second ITR and a gene operably linked to a promoter (as described herein). In some embodiments, the payload comprises a nucleic acid molecule encoding 5′-ITR-promoter-gene-ITR-3′, where the gene is a gene for AAV delivery. In some embodiments, the gene is a fluorescent protein. In some embodiments, the gene is a green fluorescent protein. In some embodiments, the payload sequence comprises a multiple cloning site.

### IV. Kits

In some aspects, the disclosure relates to kits for AAV production. In some embodiments, a kit comprises one or more polynucleic acids collectively comprising an AAV production system described herein. In some embodiments, a kit comprises an engineered cell described herein.

In some embodiments, a kit comprises a polynucleotide comprising, from 5′ to 3′: (i) a nucleic acid sequence of a 5′ inverted terminal repeat; (ii) a multiple cloning site; and (iii) a nucleic acid sequence of a 3′ inverted terminal repeat. In some embodiments, the polynucleotide is a plasmid or a vector.

The central nucleic acid of a transfer polynucleic acid may comprise a nucleic acid sequence of a multiple cloning site. Exemplary multiple cloning sites are known to those having ordinary skill in the art. A multiple cloning site can be used for cloning a payload molecule (or gene of interest)—or an expression cassette encoding a payload molecule-into the transfer polynucleic acid prior to the generation of viral vectors in a host cell.

In some embodiments, a kit further comprises a small molecule inducer corresponding to a chemically inducible promoter of the AAV production system. In some embodiments, a small molecule inducer is doxycycline, vanillate, phloretin, rapamycin, abscisic acid, gibberellic acid acetoxymethyl ester, and cumate. In some embodiments, the kits may further comprise instructions for use of the cells.

In some embodiments, a kit comprises an engineered cell, wherein the engineered cell comprises the stably integrated nucleic acid molecules of an AAV production system.

In some embodiments, a kit comprises a polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the AAV production system, optionally wherein an engineered cell comprises the polynucleic acid comprising the nucleic acid sequence of the transcriptional activator. In some embodiments, the transcriptional activator is selected from the group consisting of TetOn-3G, TetOn-V16, TetOff-Advanced, VanR-VP16, TtgR-VP16, PhlF-VP16, and the cumate cTA and rcTA.

### V. Methods of AAV Production

In some aspects, the disclosure provides methods of producing AAVs using an engineered cell or kit described herein.

In some embodiments, a method for AAV production utilizes an engineered cell comprising a nucleic acid sequence encoding for one or more of the following operably linked to an inducible promoter: Rep52, DA-Rep52, Rep40, or DA-Rep40; Rep78, DA-Rep78, Rep68, or DA-Rep68; E2A or DA-E2A; E4ORF6 or DA-E4ORF6; VARNA or DA-VARNA; VP1 or DA-VP1; VP2 or DA-VP2; VP3 or DA-VP3; AAP or DA-AAP; MAAP or DA-MAAP; and L4 100K or DA-L4 100K; and a Base Editor. In some embodiments, the inducible promoter is a chemically inducible promoter, and wherein the engineered cell further comprises a nucleic acid sequence encoding for a transcriptional activator that binds to the inducible promoter in the presence of a small molecule inducer. In some embodiments, the method for AAV production comprises contacting the cell with the small molecule inducer. In some embodiments, the small molecule inducer is doxycycline, vanillate, phloretin, rapamycin, abscisic acid, gibberellic acid acetoxymethyl ester, or cumate.

In some embodiments, a method for AAV production comprises expressing in an engineered cell described herein one or more single guide RNA(s) capable of targeting a Base Editor to a mutated start codon of DA-Rep52, DA-Rep40, DA-Rep78, DA-Rep68, DA-E2A, DA-E4ORF6, DA-VP1, DA-VP2, DA-VP3, DA-AAP, DA-MAAP, or DA-L4 100K, wherein the engineered cell comprises one or more polynucleotides encoding the one or more single guide RNA(s). In some embodiments, the one or more polynucleotides encoding the one or more single guide RNA(s) are stably integrated.

In some embodiments, a method for AAV production comprises contacting an engineered cell described herein with one or more single guide RNA(s) capable of targeting the Base Editor to a mutated start codon of DA-Rep52, DA-Rep40, DA-Rep78, DA-Rep68, DA-E2A, DA-E4ORF6, DA-VP1, DA-VP2, DA-VP3, DA-AAP, DA-MAAP, or DA-L4 100K such that that engineered cell internalizes the one or more single guide RNA(s).

In some embodiments, a method for AAV production comprises expressing in an engineered cell described herein one or more single guide RNA(s) capable of targeting a Base Editor to a non-synonymous codon of DA-Rep52, DA-Rep40, DA-Rep78, DA-Rep68, DA-E2A, DA-E4ORF6, DA-VP1, DA-VP2, DA-VP3, DA-AAP, DA-MAAP, or DA-L4 100K, wherein the engineered cell comprises one or more polynucleotides encoding the one or more single guide RNA(s). In some embodiments, the one or more polynucleotides encoding the one or more single guide RNA(s) are stably integrated.

In some embodiments, a method for AAV production comprises contacting an engineered cell described herein with one or more single guide RNA(s) capable of targeting the Base Editor to a non-synonymous codon of DA-Rep52, DA-Rep40, DA-Rep78, DA-Rep68, DA-E2A, DA-E4ORF6, DA-VP1, DA-VP2, DA-VP3, DA-AAP, DA-MAAP, or DA-L4 100K such that that engineered cell internalizes the one or more single guide RNA(s).

In some embodiments, a method for AAV production comprises expressing in an engineered cell described herein one or more single guide RNA(s) capable of targeting a Base Editor to a premature stop codon of DA-Rep52, DA-Rep40, DA-Rep78, DA-Rep68, DA-E2A, DA-E4ORF6, DA-VP1, DA-VP2, DA-VP3, DA-AAP, DA-MAAP, or DA-L4 100K, wherein the engineered cell comprises one or more polynucleotides encoding the one or more single guide RNA(s). In some embodiments, the one or more polynucleotides encoding the one or more single guide RNA(s) are stably integrated.

In some embodiments, a method for AAV production comprises contacting an engineered cell described herein with one or more single guide RNA(s) capable of targeting the Base Editor to a premature stop codon of DA-Rep52, DA-Rep40, DA-Rep78, DA-Rep68, DA-E2A, DA-E4ORF6, DA-VP1, DA-VP2, DA-VP3, DA-AAP, DA-MAAP, or DA-L4 100K such that that engineered cell internalizes the one or more single guide RNA(s).

In some embodiments, the method comprises growing the engineered cell to a confluency that is optimal for AAV production. An optimal confluency will be dependent on the type of cell the engineered cell is derived from. The skilled person will know or be able to determine the optimal confluency for AAV production.

In some embodiments, the method comprises harvesting the AAV produced from the culture of engineered cells using methods that are well known to those of skill in the art.

### EXAMPLES

### Example 1

### Base Editor AAV

### Description of Approach and Genetic Schematic:

E1 activation of cytotoxic genes in HEK293T producer lines can be avoided by reversibly disabling those genes with a premature stop codon or mutated start codon. When protein expression is desired, an Adenine Base Editor (ABE) can perform a targeted A-to-G point mutation to revert the premature stop codon to a coding amino acid. Premature stop mutations made to tryptophan (W) codons on the sense strand can be reverted by both DNA-based Cas9 ABEs and RNA-based Cas13 ABEs. On the anti-sense strand, DNA-based Cas9 ABEs can revert premature stop codons made to glutamine (Q) and arginine (R) residues. A Cytosine Base Editor (CBE) can perform a targeted C-to-T point mutation to revert the mutated start codon to methionine (M). Mutated start codons made to threonine (T) on the sense strand can be reverted by both DNA-based Cas9 CBEs and RNA-based Cas13 CBEs. On the anti-sense strand, DNA-based Cas9 CBEs can revert premature stop codons made valine (V) residues.

It was hypothesized that mutated start codons and non-synonymous mutations introduced to Rep, E2A, and E4 would prevent expression of these proteins, resulting in reduced AAV titers, improved cell health, and therefore improved ability to make stable AAV producer cells. When production of AAV is desired, the CBE can be expressed (e.g., by an inducible promoter upon treatment with a small molecule). For example, the tetracycline responsive elements (TRE) could induce expression of a CBE in the presence of doxycycline and a reverse tetracycline transactivator (rtTA). Single guide RNAs for the CBE are constitutively expressed by an RNA PolIII promoter, such as U6.

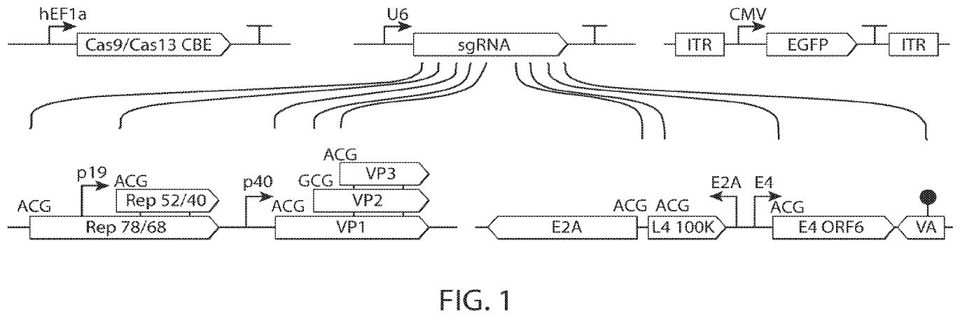

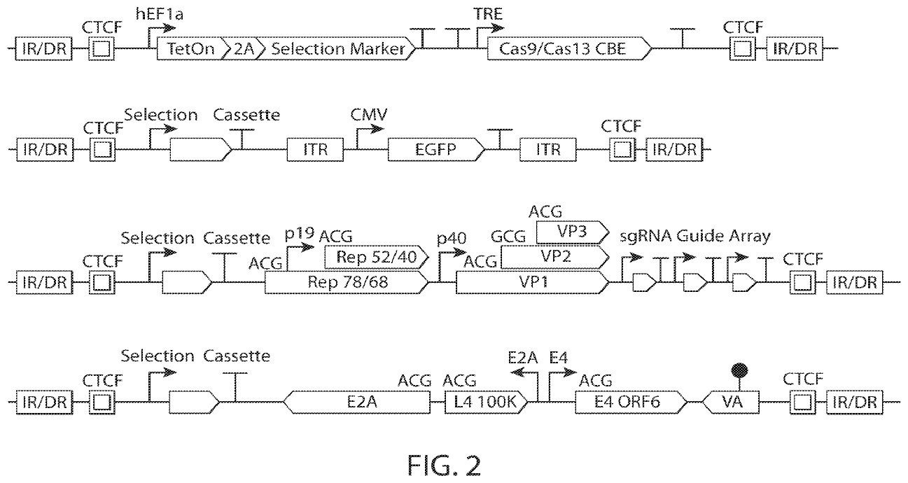

In an assay of cytosine base editing, mutated start codons and/or non-synonymous codons could be made to the pRepCap and pHelper standard plasmids (FIG. 1). Production of AAV with transient transfection would be performed with these modified plasmids to determine the impact of the mutated codons on AAV titer. Furthermore, it is expected that this result will hold when assaying the stable plasmid system (FIG. 2), displaying inducibility of AAV titers in the presence of doxycycline.

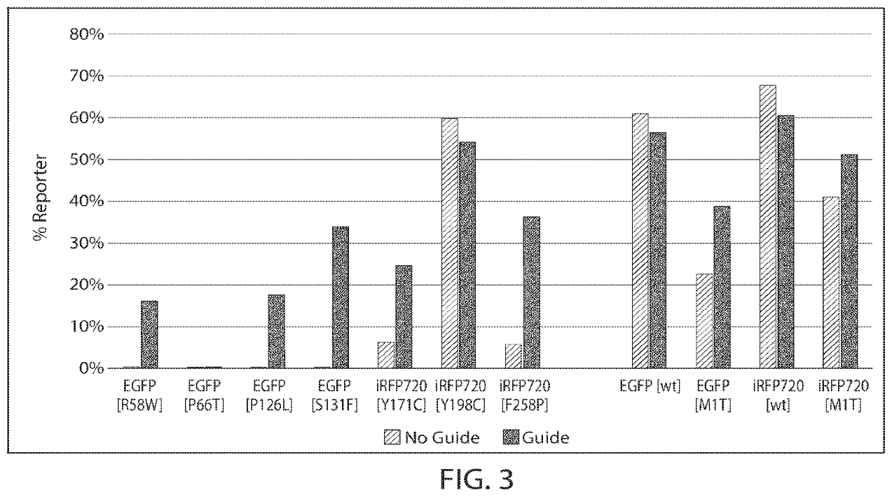

As a proxy for AAV production, the use of a CBE to revert start codon and non-synonymous mutations was tested in the fluorescent proteins EGFP and iRFP720. Production of the florescent proteins was performed with these mutated plasmids. Start codon and non-synonymous mutations were enough to diminish protein activity in most cases, almost to undetectable levels in the case of some non-synonymous mutations (FIG. 3). Mutations made to EGFP and iRFP720 could be recovered in most cases with co-transfection of a CBE and single guide RNA plasmid. Mutations to the start codon of both EGFP and iRFP720 also reduced activity, but showed higher background levels. The mutation Y198C in iRFP720 did not abolish function, and the mutation P66T in EGFP could not be recovered by a CBE and guide RNA co-transfection. These results demonstrate that mutated start codons and non-synonymous mutations can be used to control protein expression using a base editor.

When applied to AAV, the CBE was able to target the mutated start codons and induce production. Individual plasmids containing Rep, E2A, and E4 ORF6 were mutated to replace the ATG start codon with an attenuated ACG start codon. Both the set of mutated and unmutated plasmids were co-transfected with the AncBE4max CBE and either targeting or non-targeting guide RNAs. The start codon mutations reduced production of AAV to levels comparable to the negative untransfected control in the absence of targeting CBE guide RNAs. When the targeting guide RNAs were co-transfected, AAV titers were restored to the same level as the plasmids without start codon mutations. A traditional triple-transfection performed similarly to the individual Rep, E2A, and E4 ORF6 plasmids. These results demonstrate that expression of a base editor can be used to control AAV production in vivo.

### Preliminary Data and Experiment Description:

Adherent HEK293FT cells were co-transfected with either an EGFP or iRFP720 expression plasmid, CBE plasmid, and single guide RNA plasmid as a proxy for the AAV inducible system. Additionally, a second fluorescent protein plasmid that did not conflict with the spectrum of the tested protein was also co-transfected as a control for transfection activity, either TagBFP or iRFP720. Mutant variants of EGFP and iRFP720 replaced the ‘wild type’ plasmids to test their impact on fluorescence (FIG. 3). A CBE and corresponding guide were co-transfected to determine if the CBE could restore protein activity. In samples where the function of the CBE was not desired, a non-targeting guide plasmid was co-transfected to keep the amount of transfected DNA the same. Control samples containing only ‘wild type’ EGFP and iRFP720 plasmids or a negative control transfection mix without DNA were also prepared. 72 hours after transfection, cells were harvested by trypsinization and the percentage of EGFP or iRFP720 positive cells was determined by flow cytometry and used to calculate the activity of each fluorescent protein in the presence of a CBE with or without a targeting guide RNA expression plasmid.

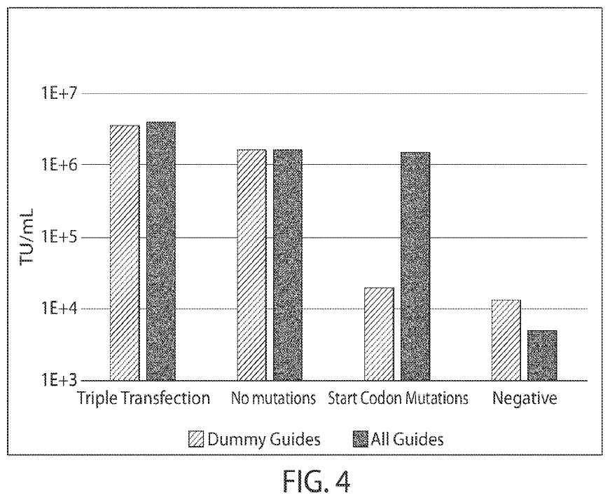

For the viral start codon assay, transfection was performed similarly to the above. Adherent HEK293FT cells were co-transfected with EGFP-expressing transfer plasmid, pRepCap, pE2A, pE4ORF6, CBE plasmid, and guide RNA plasmids. Start codon mutant variants of pRepCap, pE2A, and pE4ORF6 replaced the ‘wild type’ plasmids to test their impact on AAV titer. A CBE and corresponding guide were co-transfected to determine if the CBE could restore viral titer. In samples where the CBE activity was unwanted, a non-targeting guide was co-transfected to keep the amount of transfected DNA the same. Control samples containing only ‘wild type’ AAV2 pRepCap and pHelper plasmids or a negative control transfection mix without DNA were also prepared. 48 hours after transfection, AAV was harvested by four freeze thaw cycles in a dry ice isopropanol bath. Virus stock was transduced by addition of 10, 1, and 0.5 μL to 5e4 HEK293FT cells plated in a 96-well plate. 48 hours after transduction, transduced cells were harvested and percentage of EGFP positive cells was determined by flow cytometry and used to calculate transducing units per mL (TU/mL, FIG. 4).

TABLE 3
Nucleic Acid and Polypeptide Sequences

| SEQ ID | | |
|---|---|---|
| NO: | Descrip. | Sequence |
| 1 | pTREtight | ctcgagtttactccctatcagtgatagagaacgtatgtcgagtttactccctatcagtgatagagaacgatgtcgagtttac |
| | | tccctatcagtgatagagaacgtatgtcgagtttactccctatcagtgatagagaacgtatgtcgagtttactccctatcagt |
| | | gatagagaacgtatgtcgagtttatccctatcagtgatagagaacgtatgtcgagtttactccctatcagtgatagagaac |
| | | gtatgtcgaggtaggcgtgtacggtgggaggcctatataagcagagctcgtttagtgaaccgtcagatcgcctggaga |
| | | attcgagctcggtacccgggga |
| 2 | pTRE3G | gtttactccctatcagtgatagagaacgtatgaagagtttactccctatcagtgatagagaacgtatgcagactttactccc |
| | | tatcagtgatagagaacgtataaggagtttactccctatcagtgatagagaacgtatgaccagtttactccctatcagtgata |
| | | gagaacgtatctacagtttactccctatcagtgatagagaacgtatatccagtttactccctatcagtgatagagaacgtat |
| | | aagctttaggcgtgtacggtgggcgcctataaaagcagagctcgtttagtgaaccgtcagatcgcctggagcaattcca |
| | | caacacttttgtcttataccaactttccgtaccacttcctaccctcgtaaagtcgacaccggggcccagatctatcgatcgg |
| | | ccggataacgccacc |
| 3 | bi-TRE3G | gaattctccaggcgatctgacggttcactaaacgagctctgcttatataggcctcccaccgtacacgccacctcgacata |
| | | ctcgagtttactccctatcagtgatagagaacgtatgaagagtttactccctatcagtgatagagaacgtatgcagacttta |
| | | ctccctatcagtgatagagaacgtataaggagtttactccctatcagtgatagagaacgtatgaccagtttactccctatca |
| | | gtgatagagaacgtatctacagtttactccctatcagtgatagagaacgtatatccagtttactccctatcagtgatagaga |
| | | acgtataagctttaggcgtgtacggtgggcgcctataaaagcagagctcgtttagtgaaccgtcagatcgcctggagca |
| | | attccacaacacttttgtcttataccaactttccgtaccacttcctaccctcgtaaagtcgacaccggggcccagatctccg |
| | | cggggatcc |
| 4 | IRES | cccctctccctcccccccccctaacgttactggccgaagccgcttggaataaggccggtgtgcgtttgtctatatgttattt |
| | | tccaccatattgccgtcttttggcaatgtgagggcccggaaacctggccctgtcttcttgacgagcattcctaggggtcttt |
| | | cccctctcgccaaaggaatgcaaggtctgttgaatgtcgtgaaggaagcagttcctctggaagcttcttgaagacaaac |
| | | aacgtctgtagcgaccctttgcaggcagcggaaccccccacctggcgacaggtgcctctgcggccaaaagccacgtg |
| | | tataagatacacctgcaaaggcggcacaaccccagtgccacgttgtgagttggatagttgtggaaagagtcaaatggct |
| | | ctcctcaagcgtattcaacaaggggctgaaggatgcccagaaggtaccccattgtatgggatctgatctggggcctcgg |
| | | tgcacatgctttacatgtgtttagtcgaggttaaaaaaacgtctaggccccccgaaccacggggacgtggttttcctttga |
| | | aaaacacgatgataatatg |
| 5 | attenuated | cccccccccctaacgttactggccgaagccgcttggaataaggccggtgtgcgtttgtctatatgttattttccaccatatt |
| | IRES | gccgtcttttggcaatgtgagggcccggaaacctggccctgtcttcttgacgagcattcctaggggtctttcccctctcgc |
| | | caaaggaatgcaaggtctgttgaatgtcgtgaaggaagcagttcctctggaagcttcttgaagacaaacaacgtctgtag |
| | | cgaccctttgcaggcagcggaaccccccacctggcgacaggtgcctctgcggccaaaagccacgtgtataagataca |
| | | cctgcaaaggcggcacaaccccagtgccacgttgtgagttggatagttgtggaaagagtcaaatggctctcctcaagc |
| | | gtattcaacaaggggctgaaggatgcccagaaggtaccccattgtatgggatctgatctggggcctcggtgcacatgct |
| | | ttacatgtgtttagtcgaggttaaaaaaacgtctaggccccccgaaccacggggacgtggttttcctttgaaaaacacgat |
| | | gataatagttatc |
| 6 | Rep52 (wt) | MELVGWLVDKGITSEKQWIQEDQASYISFNAASNSRSQIKAALDNAGKIMS |
| | | LTKTAPDYLVGQQPVEDISSNRIYKILELNGYDPQYAASVFLGWATKKFGK |
| | | RNTIWLFGPATTGKTNIAEAIAHTVPFYGCVNWTNENFPFNDCVDKMVIW |
| | | WEEGKMTAKVVESAKAILGGSKVRVDQKCKSSAQIDPTPVIVTSNTNMCA |
| | | VIDGNSTTFEHQQPLQDRMFKFELTRRLDHDFGKVTKQEVKDFFRWAKDH |
| | | VVEVEHEFYVKKGGAKKRPAPSDADISEPKRVRESVAQPSTSDAEASINYA |
| | | DRYQNKCSRHVGMNLMLFPCRQCERMNQNSNICFTHGQKDCLECFPVSES |
| | | QPVSVVKKAYQKLCYIHHIMGKVPDACTACDLVNVDLDDCIFEQ |
| 7 | Rep40 (wt) | MELVGWLVDKGITSEKQWIQEDQASYISFNAASNSRSQIKAALDNAGKIMS |
| | | LTKTAPDYLVGQQPVEDISSNRIYKILELNGYDPQYAASVFLGWATKKFGK |
| | | RNTIWLFGPATTGKTNIAEAIAHTVPFYGCVNWTNENFPFNDCVDKMVIW |
| | | WEEGKMTAKVVESAKAILGGSKVRVDQKCKSSAQIDPTPVIVTSNTNMCA |
| | | VIDGNSTTFEHQQPLQDRMFKFELTRRLDHDFGKVTKQEVKDFFRWAKDH |
| | | VVEVEHEFYVKKGGAKKRPAPSDADISEPKRVRESVAQPSTSDAEASINYA |
| | | DRLARGHSL |
| 8 | Rep78 (wt) | MPGFYEIVIKVPSDLDEHLPGISDSFVNWVAEKEWELPPDSDMDLNLIEQAP |
| | | LTVAEKLQRDFLTEWRRVSKAPEALFFVQFEKGESYFHMHVLVETTGVKS |
| | | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYMELVGWLVDKGITSEKQWIQEDQASYISFN |
| | | AASNSRSQIKAALDNAGKIMSLTKTAPDYLVGQQPVEDISSNRIYKILELNG |
| | | YDPQYAASVFLGWATKKFGKRNTIWLFGPATTGKTNIAEAIAHTVPFYGC |
| | | VNWTNENFPFNDCVDKMVIWWEEGKMTAKVVESAKAILGGSKVRVDQK |
| | | CKSSAQIDPTPVIVTSNTNMCAVIDGNSTTFEHQQPLQDRMFKFELTRRLDH |
| | | DFGKVTKQEVKDFFRWAKDHVVEVEHEFYVKKGGAKKRPAPSDADISEP |
| | | KRVRESVAQPSTSDAEASINYADRYQNKCSRHVGMNLMLFPCRQCERMN |
| | | QNSNICFTHGQKDCLECFPVSESQPVSVVKKAYQKLCYIHHIMGKVPDACT |
| | | ACDLVNVDLDDCIFEQ |
| 9 | Rep68 (wt) | MPGFYEIVIKVPSDLDEHLPGISDSFVNWVAEKEWELPPDSDMDLNLIEQAP |
| | | LTVAEKLQRDFLTEWRRVSKAPEALFFVQFEKGESYFHMHVLVETTGVKS |
| | | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYMELVGWLVDKGITSEKQWIQEDQASYISFN |
| | | AASNSRSQIKAALDNAGKIMSLTKTAPDYLVGQQPVEDISSNRIYKILELNG |
| | | YDPQYAASVFLGWATKKFGKRNTIWLFGPATTGKTNIAEAIAHTVPFYGC |
| | | VNWTNENFPFNDCVDKMVIWWEEGKMTAKVVESAKAILGGSKVRVDQK |
| | | CKSSAQIDPTPVIVTSNTNMCAVIDGNSTTFEHQQPLQDRMFKFELTRRLDH |
| | | DFGKVTKQEVKDFFRWAKDHVVEVEHEFYVKKGGAKKRPAPSDADISEP |
| | | KRVRESVAQPSTSDAEASINYADRLARGHSL |
| 10 | E2A (wt) | MASREEEQRETTPERGRGAARRPPTMEDVSSPSPSPPPPRAPPKKRLRRRLE |
| | | SEDEEDSSQDALVPRTPSPRPSTSTADLAIASKKKKKRPSPKPERPPSPEVIV |
| | | DSEEEREDVALQMVGFSNPPVLIKHGKGGKRTVRRLNEDDPVARGMRTQE |
| | | EKEESSEAESESTVINPLSLPIVSAWEKGMEAARALMDKYHVDNDLKANFK |
| | | LLPDQVEALAAVCKTWLNEEHRGLQLTFTSNKTFVTMMGRFLQAYLQSFA |
| | | EVTYKHHEPTGCALWLHRCAEIEGELKCLHGSIMINKEHVIEMDVTSENGQ |
| | | RALKEQSSKAKIVKNRWGRNVVQISNTDARCCVHDAACPANQFSGKSCG |
| | | MFFSEGAKAQVAFKQIKAFMQALYPNAQTGHGHLLMPLRCECNSKPGHAP |
| | | FLGRQLPKLTPFALSNAEDLDADLISDKSVLASVHHPALIVFQCCNPVYRNS |
| | | RAQGGGPNCDFKISAPDLLNALVMVRSLWSENFTELPRMVVPEFKWSTKH |
| | | QYRNVSLPVAHSDARQNPFDF |
| 11 | E4 ORF6 | MTTSGVPFGMTLRPTRSRLSRRTPYSRDRLPPFETETRATILEDHPLLPECNT |
| | (wt) | LTMHNVSYVRGLPCSVGFTLIQEWVVPWDMVLTREELVILRKCMHVCLCC |
| | | ANIDIMTSMMIHGYESWALHCHCSSPGSLQCIAGGQVLASWFRMVVDGA |
| | | MFNQRFIWYREVVNYNMPKEVMFMSSVFMRGRHLIYLRLWYDGHVGSV |
| | | VPAMSFGYSALHCGILNNIVVLCCSYCADLSEIRVRCCARRTRRLMLRAVR |
| | | IIAEETTAMLYSCRTERRRQQFIRALLQHHRPILMHDYDSTPM |
| 12 | E4 ORF6 | atgactacgtccggcgttccatttggcatgacactacgaccaacacgatctcggttgtctcggcgcactccgtacagtag |
| | (splice | ggatcgcctacctccttttgagacagagacccgcgctaccatactggaggatcatccgctgctgcccgaatgtaacactt |
| | site | tgacaatgcacaaTgtTTCCtacgtgcgaggtcttccctgcagtgtgggatttacgctgattcaggaatgggttgttcc |
| | removed) | ctgggatatggttctgacgcgggaggagcttgtaatcctgaggaagtgtatgcacgtgtgcctgtgttgtgccaacattg |
| | | atatcatgacgagcatgatgatccatggttacgagtcctgggctctccactgtcattgttccagtcccggttccctgcagtg |
| | | catagccggcgggcaggttttggccagctggtttaggatggtggtggatggcgccatgtttaatcagaggtttatatggt |
| | | accgggaggtggtgaattacaacatgccaaaagaggtaatgtttatgtccagcgtgtttatgaggggtcgccacttaatct |
| | | acctgcgcttgtggtatgatggccacgtgggttctgtggtccccgccatgagctttggatacagcgccttgcactgtggg |
| | | attttgaacaatattgtggtgctgtgctgcagttactgtgctgatttaagtgagatcagggtgcgctgctgtgcccggagga |
| | | caaggcgtctcatgctgcgggcggtgcgaatcatcgctgaggagaccactgccatgttgtattcctgcaggacggagc |
| | | ggcggcggcagcagtttattcgcgcgctgctgcagcaccaccgccctatcctgatgcacgattatgactctacccccat |
| | | gTAGtaa |
| 13 | VARNA | CGACGTAATCCGTAGATGTACCTGGACATCCAGGTGATGCCGGCGGCG |
| | | GTGGTGGAGGCGCGCGGAAAGTCGCGGACGCGGTTCCAGATGTTGCGC |
| | | AGCGGCAAAAAGTGCTCCATGGTCGGGACGCTCTGGCCGGTGAGGCGT |
| | | GCGCAGTCGTTGACGCTCTAGACCGTGCAAAAGGAGAGCCTGTAAGCG |
| | | GGCACTCTTCCGTGGTCTGGTGGATAAATTCGCAAGGGTATCATGGCGG |
| | | ACGACCGGGGTTCGAACCCCGGATCCGGCCGTCCGCCGTGATCCATGCG |
| | | GTTACCGCCCGCGTGTCGAACCCAGGTGTGCGACGTCAGACAACGGGG |
| | | GAGCGCTCCTTTTGGCTTCCTTCCAGGCGCGGCGGCTGCTGCGCTAGCTT |
| | | TTTTGGCCACTGGCCGCGCGCGGCGTAAGCGGTTAGGCTGGAAAGCGA |
| | | AAGCATTAAGTGGCTCGCTCCCTGTAGCCGGAGGGTTATTTTCCAAGGG |
| | | TTGAGTCGCAGGACCCCCGGTTCGAGTCTCGGGCCGGCCGGACTGCGGC |
| | | GAACGGGGGTTTGCCTCCCCGTCATGCAAGACCCCGCTTGCAAATTCCT |
| | | CCGGAAACAGGGACGAGCCCCTTTTTTGCTTTTCCCAGATGCATCCGGT |
| | | GCTGCGGCAGATGCGCCCCCCTCCTCAGCAGCGGCAAGAGCAAGAGCA |
| | | GCGGCAGACATGCAGGGCACCCTCCCCTTCTCCTACCGCGTCAGGAGGG |
| | | GCAACATCC |
| 14 | VP1 (wt) | MAADGYLPDWLEDTLSEGIRQWWKLKPGPPPPKPAERHKDDSRGLVLPGY |
| | | KYLGPFNGLDKGEPVNEADAAALEHDKAYDRQLDSGDNPYLKYNHADAE |
| | | FQERLKEDTSFGGNLGRAVFQAKKRVLEPLGLVEEPVKTAPGKKRPVEHSP |
| | | VEPDSSSGTGKAGQQPARKRLNFGQTGDADSVPDPQPLGQPPAAPSGLGTN |
| | | TMATGSGAPMADNNEGADGVGNSSGNWHCDSTWMGDRVITTSTRTWAL |
| | | PTYNNHLYKQISSQSGASNDNHYFGYSTPWGYFDFNRFHCHFSPRDWQRLI |
| | | NNNWGFRPKRLNFKLFNIQVKEVTQNDGTTTIANNLTSTVQVFTDSEYQLP |
| | | YVLGSAHQGCLPPFPADVFMVPQYGYLTLNNGSQAVGRSSFYCLEYFPSQ |
| | | MLRTGNNFTFSYTFEDVPFHSSYAHSQSLDRLMNPLIDQYLYYLSRTNTPS |
| | | GTTTQSRLQFSQAGASDIRDQSRNWLPGPCYRQQRVSKTSADNNNSEYSW |
| | | TGATKYHLNGRDSLVNPGPAMASHKDDEEKFFPQSGVLIFGKQGSEKTNV |
| | | DIEKVMITDEEEIRTTNPVATEQYGSVSTNLQRGNRQAATADVNTQGVLPG |
| | | MVWQDRDVYLQGPIWAKIPHTDGHFHPSPLMGGFGLKHPPPQILIKNTPVP |
| | | ANPSTTFSAAKFASFITQYSTGQVSVEIEWELQKENSKRWNPEIQYTSNYNK |
| | | SVNVDFTVDTNGVYSEPRPIGTRYLTRNL |
| 15 | VP2 (wt) | TAPGKKRPVEHSPVEPDSSSGTGKAGQQPARKRLNFGQTGDADSVPDPQPL |
| | | GQPPAAPSGLGTNTMATGSGAPMADNNEGADGVGNSSGNWHCDSTWMG |
| | | DRVITTSTRTWALPTYNNHLYKQISSQSGASNDNHYFGYSTPWGYFDFNRF |
| | | HCHFSPRDWQRLINNNWGFRPKRLNFKLFNIQVKEVTQNDGTTTIANNLTS |
| | | TVQVFTDSEYQLPYVLGSAHQGCLPPFPADVFMVPQYGYLTLNNGSQAVG |
| | | RSSFYCLEYFPSQMLRTGNNFTFSYTFEDVPFHSSYAHSQSLDRLMNPLIDQ |
| | | YLYYLSRTNTPSGTTTQSRLQFSQAGASDIRDQSRNWLPGPCYRQQRVSKT |
| | | SADNNNSEYSWTGATKYHLNGRDSLVNPGPAMASHKDDEEKFFPQSGVLI |
| | | FGKQGSEKTNVDIEKVMITDEEEIRTTNPVATEQYGSVSTNLQRGNRQAAT |
| | | ADVNTQGVLPGMVWQDRDVYLQGPIWAKIPHTDGHFHPSPLMGGFGLKH |
| | | PPPQILIKNTPVPANPSTTFSAAKFASFITQYSTGQVSVEIEWELQKENSKRW |
| | | NPEIQYTSNYNKSVNVDFTVDTNGVYSEPRPIGTRYLTRNL |
| 16 | VP3 (wt) | MATGSGAPMADNNEGADGVGNSSGNWHCDSTWMGDRVITTSTRTWALP |
| | | TYNNHLYKQISSQSGASNDNHYFGYSTPWGYFDFNRFHCHFSPRDWQRLI |
| | | NNNWGFRPKRLNFKLFNIQVKEVTQNDGTTTIANNLTSTVQVFTDSEYQLP |
| | | YVLGSAHQGCLPPFPADVFMVPQYGYLTLNNGSQAVGRSSFYCLEYFPSQ |
| | | MLRTGNNFTFSYTFEDVPFHSSYAHSQSLDRLMNPLIDQYLYYLSRTNTPS |
| | | GTTTQSRLQFSQAGASDIRDQSRNWLPGPCYRQQRVSKTSADNNNSEYSW |
| | | TGATKYHLNGRDSLVNPGPAMASHKDDEEKFFPQSGVLIFGKQGSEKTNV |
| | | DIEKVMITDEEEIRTTNPVATEQYGSVSTNLQRGNRQAATADVNTQGVLPG |
| | | MVWQDRDVYLQGPIWAKIPHTDGHFHPSPLMGGFGLKHPPPQILIKNTPVP |
| | | ANPSTTFSAAKFASFITQYSTGQVSVEIEWELQKENSKRWNPEIQYTSNYNK |
| | | SVNVDFTVDTNGVYSEPRPIGTRYLTRNL |
| 17 | AAP (wt) | LETQTQYLTPSLSDSHQQPPLVWELIRWLQAVAHQWQTITRAPTEWVIPREI |
| | | GIAIPHGWATESSPPAPEPGPCPPTTTTSTNKFPANQEPRTTITTLATAPLGGI |
| | | LTSTDSTATFHHVTGKDSSTTTGDSDPRDSTSSSLTFKSKRSRRMTVRRRLPI |
| | | TLPARFRCLLTRSTSSRTSSARRIKDASRRSQQTSSWCHSMDTSP |
| 18 | P2A | ATNFSLLKQAGDVEENPGP |
| | (without | |
| | GSG) | |
| 19 | T2A | EGRGSLLTCGDVEENPGP |
| | (without | |
| | GSG) | |
| 20 | WT Rep | atgccggggttttacgagattgtgattaaggtccccagcgaccttgacgagcatctgcccggcatttctgacagctttgtg |
| | | aactgggtggccgagaaggaatgggagttgccgccagattctgacatggatctgaatctgattgagcaggcacccctg |
| | | accgtggccgagaagctgcagcgcgactttctgacggaatggcgccgtgtgagtaaggccccggaggcccttttcttt |
| | | gtgcaatttgagaagggagagagctacttccacatgcacgtgctcgtggaaaccaccggggtgaaatccatggttttgg |
| | | gacgtttcctgagtcagattcgcgaaaaactgattcagagaatttaccgcgggatcgagccgactttgccaaactggttc |
| | | gcggtcacaaagaccagaaatggcgccggaggcgggaacaaggtggtggatgagtgctacatccccaattacttgct |
| | | ccccaaaacccagcctgagctccagtgggcgtggactaatatggaacagtatttaagcgcctgtttgaatctcacggag |
| | | cgtaaacggttggtggcgcagcatctgacgcacgtgtcgcagacgcaggagcagaacaaagagaatcagaatccca |
| | | attctgatgcgccggtgatcagatcaaaaacttcagccaggtacatggagctggtcgggtggctcgtggacaagggga |
| | | ttacctcggagaagcagtggatccaggaggaccaggcctcatacatctccttcaatgcggcctccaactcgcggtccc |
| | | aaatcaaggctgccttggacaatgcgggaaagattatgagcctgactaaaaccgcccccgactacctggtgggccag |
| | | cagcccgtggaggacatttccagcaatcggatttataaaattttggaactaaacgggtacgatccccaatatgcggcttc |
| | | cgtctttctgggatgggccacgaaaaagttcggcaagaggaacaccatctggctgtttgggcctgcaactaccgggaa |
| | | gaccaacatcgcggaggccatagcccacactgtgcccttctacgggtgcgtaaactggaccaatgagaactttcccttc |
| | | aacgactgtgtcgacaagatggtgatctggtgggaggaggggaagatgaccgccaaggtcgtggagtcggccaaag |
| | | ccattctcggaggaagcaaggtgcgcgtggaccagaaatgcaagtcctcggcccagatagacccgactcccgtgatc |
| | | gtcacctccaacaccaacatgtgcgccgtgattgacgggaactcaacgaccttcgaacaccagcagccgttgcaagac |
| | | cggatgttcaaatttgaactcacccgccgtctggatcatgactttgggaaggtcaccaagcaggaagtcaaagactttttc |
| | | cggtgggcaaaggatcacgtggttgaggtggagcatgaattctacgtcaaaaagggtggagccaagaaaagacccg |
| | | cccccagtgacgcagatataagtgagcccaaacgggtgcgcgagtcagttgcgcagccatcgacgtcagacgcgga |
| | | agcttcgatcaactacgcagacaggtaccaaaacaaatgttctcgtcacgtgggcatgaatctgatgctgtttccctgca |
| | | gacaatgcgagagaatgaatcagaattcaaatatctgcttcactcacggacagaaagactgtttagagtgctttcccgtgt |
| | | cagaatctcaacccgtttctgtcgtcaaaaaggcgtatcagaaactgtgctacattcatcatatcatgggaaaggtgccag |
| | | acgcttgcactgcctgcgatctggtcaatgtggatttggatgactgcatctttgaacaataaatgatttaaatcaggtatg |
| | | gctgccgatggttatcttccagattggctcgaggacactctctctga |
| 21 | Rep78 + 52 | cTGGCGGGCTTCTACGAGATCGTGATCAAGGTGCCCAGCGACCTGGACG |
| | Only | AGCATCTGCCTGGCATCAGCGACAGCTTCGTGAATTGGGTCGCCGAGAA |
| | | AGAGTGGGAGCTGCCTCCTGACAGCGACITGGACCTGAACCTGATTGAG |
| | | CAGGCCCCTCTGACAGTGGCCGAGAAGCTGCAGAGGGATTTCCTGACA |
| | | GAGTGGCGGAGAGTGTCTAAGGCCCCTGAGGCTCTGTTCTTCGTGCAGT |
| | | TCGAGAAGGGCGAGAGCTACTTCCACTTACACGTGCTGGTCGAGACAA |
| | | CCGGCGTGAAGTCTTTAGTGCTGGGCAGATTCCTGAGCCAGATCAGAGA |
| | | GAAGCTGATCCAGCGGATCTACCGGGGCATCGAGCCCACACTGCCTAAT |
| | | TGGTTCGCCGTGACCAAGACCAGAAACGGcGCTGGCGGCGGAAACAAG |
| | | GTGGTGGACGAGTGCTACATCCCCAACTACCTGCTGCCTAAGACACAGC |
| | | CCGAACTGCAGTGGGCCTGGACCAACTTAGAACAGTACCTGAGCGCCT |
| | | GCCTGAATCTGACCGAGCGGAAAAGACTGGTGGCCCAGCATCTGACCC |
| | | ACGTGTCCCAGACACAAGAGCAGAACAAAGAGAATCAGAACCCCAACA |
| | | GCGACGCCCCTGTGATCAGAAGCAAGACCAGCGCCAGATACATGGAAC |
| | | TCGTTGGCTGGCTGGTGGACAAGGGCATCACAAGCGAGAAGCAGTGGA |
| | | TCCAAGAGGACCAGGCCAGCTACATCAGCTTCAACGCCGCCTCCAACA |
| | | GCAGATCCCAGATCAAGGCCGCTCTGGACAACGCCGGCAAGATCATGA |
| | | GCCTGACAAAGACAGCCCCTGACTACCTCGTGGGCCAGCAGCCTGTGG |
| | | AAGATATCAGCAGCAACCGGATCTACAAGATCCTGGAACTGAACGGCT |
| | | ACGACCCTCAGTATGCCGCCTCTGTGTTTCTCGGCTGGGCTACCAAGAA |
| | | GTTCGGCAAGCGGAACACCATCTGGCTGTTTGGCCCTGCCACAACCGGC |
| | | AAGACCAATATCGCCGAGGCTATCGCCCACACCGTGCCTTTTTACGGCT |
| | | GCGTGAACTGGACCAATGAGAACTTCCCCTTCAACGACTGCGTGGACAA |
| | | GATGGTCATTTGGTGGGAAGAGGGCAAGATGACCGCCAAAGTGGTGGA |
| | | AAGCGCCAAGGCCATCCTCGGCGGATCTAAAGTTCGCGTGGACCAGAA |
| | | GTGCAAGTCTAGCGCCCAGATCGACCCCACACCTGTGATCGTGACCAGC |
| | | AACACCAACATGTGCGCCGTGATCGACGGCAACAGCACCACCTTTGAA |
| | | CACCAGCAGCCACTGCAGGACCGGATGTTCAAGTTCGAGCTGACCAGA |
| | | CGGCTGGACCACGACTTCGGCAAAGTGACCAAGCAAGAAGTGAAGGAC |
| | | TTCTTCCGCTGGGCCAAAGATCACGTGGTGGAAGTGGAACACGAGTTCT |
| | | ACGTGAAGAAAGGCGGAGCCAAGAAGAGGCCCGCTCCTTCCGATGCCG |
| | | ATATCAGCGAGCCTAAGCGCGTGCGGGAATCTGTGGCTCAGCCTAGCAC |
| | | ATCTGATGCCGAGGCCAGCATCAACTACGCCGACAGATACCAGAACAA |
| | | GTGCAGCCGGCACGTGGGAATGAATCTGATGCTGTTCCCCTGTCGGCAG |
| | | TGCGAGCGGATGAACCAGAACAGCAACATCTGCTTCACCCACGGCCAG |
| | | AAAGACTGCCTGGAATGCTTCCCCGTGTCCGAGTCTCAGCCTGTGTCCG |
| | | TGGTCAAGAAGGCCTACCAGAAGCTGTGTTACATCCACCACATCATGGG |
| | | CAAAGTGCCCGATGCCTGCACCGCCTGCGATCTGGTTAATGTGGACCTG |
| | | GATGACTGCATCTTCGAGCAGTGA |
| 22 | Rep52 IRES | GCCGCCACCATGGAATTAGTGGGCTGGTTGGTCGATAAAGGCATCACA |
| | Rep78 Only | AGCGAAAAACAATGGATTCAAGAAGATCAAGCGAGCTATATTAGTTTT |
| | | AACGCCGCTAGTAATAGCAGAAGTCAGATTAAAGCCGCTCTCGATAAC |
| | | GCCGGCAAAATCATGTCTTTAACCAAGACAGCTCCTGATTATTTAGTCG |
| | | GGCAACAACCTGTCGAGGACATCAGTTCTAACAGAATCTACAAGATCCT |
| | | CGAATTGAATGGCTATGACCCTCAGTACGCCGCCAGTGTGTTCTTAGGC |
| | | TGGGCTACCAAGAAATTTGGGAAACGCAATACAATTTGGTTATTCGGCC |
| | | CCGCCACCACAGGCAAAACAAATATTGCCGAAGCTATCGCTCATACCGT |
| | | CCCTTTCTATGGCTGTGTGAATTGGACAAACGAAAATTTCCCTTTTAATG |
| | | ATTGCGTGGATAAAATGGTCATTTGGTGGGAAGAAGGCAAAATGACAG |
| | | CTAAAGTGGTCGAAAGCGCTAAGGCTATCTTGGGCGGCTCTAAAGTCAG |
| | | AGTCGATCAAAAGTGTAAAAGTAGCGCTCAAATCGATCCCACCCCTGTC |
| | | ATTGTGACAAGTAATACAAATATGTGTGCTGTCATCGATGGCAATAGCA |
| | | CCACATTTGAGCATCAACAACCCCTCCAGGATAGAATGTTTAAGTTCGA |
| | | GTTGACAAGAAGATTAGACCACGATTTCGGCAAAGTGACAAAACAAGA |
| | | GGTGAAGGATTTCTTTAGATGGGCCAAAGACCATGTCGTGGAAGTCGA |
| | | ACACGAGTTTTATGTGAAGAAAGGCGGCGCTAAAAAGCGGCCTGCTCC |
| | | TTCCGATGCCGACATCTCCGAACCTAAGAGAGTCAGAGAAAGCGTGGC |
| | | CCAACCCAGCACCAGCGATGCCGAGGCCAGCATTAATTATGCCGATCGC |
| | | TATCAGAATAAGTGCAGCAGACATGTCGGGATGAACTTAATGTTATTCC |
| | | CTTGTCGGCAGTGTGAACGGATGAACCAAAACAGCAACATTTGTTTTAC |
| | | CCACGGACAAAAGGATTGCCTGGAATGTTTCCCTGTCAGCGAGAGCCA |
| | | GCCTGTGAGCGTGGTGAAGAAAGCCTACCAAAAGTTATGTTATATCCAC |
| | | CACATTATGGGCAAAGTCCCCGATGCCTGTACCGCTTGTGACTTAGTGA |
| | | ACGTAGACCTCGACGATTGTATTTTCGAGCAGTGAtaaGcccctctccctcccccccc |
| | | cctaacgttactggccgaagccgcttggaataaggccggtgtgcgtttgtctatatgttattttccaccatattgccgtct |
| | | tttggcaatgtgagggcccggaaacctggccctgtcttcttgacgagcattcctaggggtctttcccctctcgccaaagga |
| | | atgcaaggtctgttgaatgtcgtgaaggaagcagttcctctggaagcttcttgaagacaaacaacgtctgtagcgaccctt |
| | | tgcaggcagcggaaccccccacctggcgacaggtgcctctgcggccaaaagccacgtgtataagatacacctgcaaa |
| | | ggcggcacaaccccagtgccacgttgtgagttggatagttgtggaaagagtcaaatggctctcctcaagcgtattcaac |
| | | aaggggctgaaggatgcccagaaggtaccccattgtatgggatctgatctggggcctcggtgcacatgctttacatgtg |
| | | tttagtcgaggttaaaaaaacgtctaggccccccgaaccacggggacgtggttttcctttgaaaaacacgatgataatat |
| | | gCCTGGCTTCTACGAGATCGTGATCAAGGTGCCCAGCGACCTGGACGAG |
| | | CATCTGCCTGGCATCAGCGACAGCTTCGTGAATTGGGTCGCCGAGAAAG |
| | | AGTGGGAGCTGCCTCCTGACAGCGACATGGACCTGAACCTGATTGAGC |
| | | AGGCCCCTCTGACAGTGGCCGAGAAGCTGCAGAGGGATTTCCTGACAG |
| | | AGTGGCGGAGAGTGTCTAAGGCCCCTGAGGCTCTGTTCTTCGTGCAGTT |
| | | CGAGAAGGGCGAGAGCTACTTCCACATGCACGTGCTGGTCGAGACAAC |
| | | CGGCGTGAAGTCTATGGTGCTGGGCAGATTCCTGAGCCAGATCAGAGA |
| | | GAAGCTGATCCAGCGGATCTACCGGGGCATCGAGCCCACACTGCCTAAT |
| | | TGGTTCGCCGTGACCAAGACCAGAAACGGTGCTGGCGGCGGAAACAAG |
| | | GTGGTGGACGAGTGCTACATCCCCAACTACCTGCTGCCTAAGACACAGC |
| | | CCGAACTGCAGTGGGCCTGGACCAACATGGAACAGTACCTGAGCGCCT |
| | | GCCTGAATCTGACCGAGCGGAAAAGACTGGTGGCCCAGCATCTGACCC |
| | | ACGTGTCCCAGACACAAGAGCAGAACAAAGAGAATCAGAACCCCAACA |
| | | GCGACGCCCCTGTGATCAGAAGCAAGACCAGCGCCAGATACGGaGAAC |
| | | TCGTTGGCTGGCTGGTGGACAAGGGCATCACAAGCGAGAAGCAGTGGA |
| | | TCCAAGAGGACCAGGCCAGCTACATCAGCTTCAACGCCGCCTCCAACA |
| | | GCAGATCCCAGATCAAGGCCGCTCTGGACAACGCCGGCAAGATCATGA |
| | | GCCTGACAAAGACAGCCCCTGACTACCTCGTGGGCCAGCAGCCTGTGG |
| | | AAGATATCAGCAGCAACCGGATCTACAAGATCCTGGAACTGAACGGCT |
| | | ACGACCCTCAGTATGCCGCCTCTGTGTTTCTCGGCTGGGCTACCAAGAA |
| | | GTTCGGCAAGCGGAACACCATCTGGCTGTTTGGCCCTGCCACAACCGGC |
| | | AAGACCAATATCGCCGAGGCTATCGCCCACACCGTGCCTTTTTACGGCT |
| | | GCGTGAACTGGACCAATGAGAACTTCCCCTTCAACGACTGCGTGGACAA |
| | | GATGGTCATTTGGTGGGAAGAGGGCAAGATGACCGCCAAAGTGGTGGA |
| | | AAGCGCCAAGGCCATCCTCGGCGGATCTAAAGTTCGCGTGGACCAGAA |
| | | GTGCAAGTCTAGCGCCCAGATCGACCCCACACCTGTGATCGTGACCAGC |
| | | AACACCAACATGTGCGCCGTGATCGACGGCAACAGCACCACCTTTGAA |
| | | CACCAGCAGCCACTGCAGGACCGGATGTTCAAGTTCGAGCTGACCAGA |
| | | CGGCTGGACCACGACTTCGGCAAAGTGACCAAGCAAGAAGTGAAGGAC |
| | | TTCTTCCGCTGGGCCAAAGATCACGTGGTGGAAGTGGAACACGAGTTCT |
| | | ACGTGAAGAAAGGCGGAGCCAAGAAGAGGCCCGCTCCTTCCGATGCCG |
| | | ATATCAGCGAGCCTAAGCGCGTGCGGGAATCTGTGGCTCAGCCTAGCAC |
| | | ATCTGATGCCGAGGCCAGCATCAACTACGCCGACAGATACCAGAACAA |
| | | GTGCAGCCGGCACGTGGGAATGAATCTGATGCTGTTCCCCTGTCGGCAG |
| | | TGCGAGCGGATGAACCAGAACAGCAACATCTGCTTCACCCACGGCCAG |
| | | AAAGACTGCCTGGAATGCTTCCCCGTGTCCGAGTCTCAGCCTGTGTCCG |
| | | TGGTCAAGAAGGCCTACCAGAAGCTGTGTTACATCCACCACATCATGGG |
| | | CAAAGTGCCCGATGCCTGCACCGCCTGCGATCTGGTTAATGTGGACCTG |
| | | GATGACTGCATCTTCGAGCAGTGA |
| 23 | Cas9 ABE | MSEVEFSHEYWMRHALTLAKRAWDEREVPVGAVLVHNNRVIGEGWNRPI |
| | ABE7.10 | GRHDPTAHAEIMALRQGGLVMQNYRLIDATLYVTLEPCVMCAGAMIHSRI |
| | | GRVVFGARDAKTGAAGSLMDVLHHPGMNHRVEITEGILADECAALLSDFF |
| | | RMRRQEIKAQKKAQSSTDSGGSSGGSSGSETPGTSESATPESSGGSSGGSSE |
| | | VEFSHEYWMRHALTLAKRARDEREVPVGAVLVLNNRVIGEGWNRAIGLH |
| | | DPTAHAEIMALRQGGLVMQNYRLIDATLYVTFEPCVMCAGAMIHSRIGRV |
| | | VFGVRNAKTGAAGSLMDVLHYPGMNHRVEITEGILADECAALLCYFFRMP |
| | | RQVFNAQKKAQSSTDSGGSSGGSSGSETPGTSESATPESSGGSSGGSDKKYS |
| | | IGLAIGTNSVGWAVITDEYKVPSKKFKVLGNTDRHSIKKNLIGALLFDSGET |
| | | AEATRLKRTARRRYTRRKNRICYLQEIFSNEMAKVDDSFFHRLEESFLVEE |
| | | DKKHERHPIFGNIVDEVAYHEKYPTIYHLRKKLVDSTDKADLRLIYLALAH |
| | | MIKFRGHFLIEGDLNPDNSDVDKLFIQLVQTYNQLFEENPINASGVDAKAIL |
| | | SARLSKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTEITKAP |
| | | LSASMIKRYDEHHQDLTLLKALVRQQLPEKYKEIFFDQSKNGYAGYIDGGA |
| | | SQEEFYKFIKPILEKMDGTEELLVKLNREDLLRKQRTFDNGSIPHQIHLGEL |
| | | HAILRRQEDFYPFLKDNREKIEKILTFRIPYYVGPLARGNSRFAWMTRKSEE |
| | | TITPWNFEEVVDKGASAQSFIERMTNFDKNLPNEKVLPKHSLLYEYFTVYN |
| | | ELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTL |
| | | FEDREMIEERLKTYAHLFDDKVMKQLKRRRYTGWGRLSRKLINGIRDKQS |
| | | GKTILDFLKSDGFANRNFMQLIHDDSLTFKEDIQKAQVSGQGDSLHEHIAN |
| | | LAGSPAIKKGILQTVKVVDELVKVMGRHKPENIVIEMARENQTTQKGQKN |
| | | SRERMKRIEEGIKELGSQILKEHPVENTQLQNEKLYLYYLQNGRDMYVDQE |
| | | LDINRLSDYDVDHIVPQSFLKDDSIDNKVLTRSDKNRGKSDNVPSEEVVKK |
| | | MKNYWRQLLNAKLITQRKFDNLTKAERGGLSELDKAGFIKRQLVETRQIT |
| | | KHVAQILDSRMNTKYDENDKLIREVKVITLKSKLVSDFRKDFQFYKVREIN |
| | | NYHHAHDAYLNAVVGTALIKKYPKLESEFVYGDYKVYDVRKMIAKSEQEI |
| | | GKATAKYFFYSNIMNFFKTEITLANGEIRKRPLIETNGETGEIVWDKGRDFA |
| | | TVRKVLSMPQVNIVKKTEVQTGGFSKESILPKRNSDKLIARKKDWDPKKY |
| | | GGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYV |
| | | NFLYLASHYEKLKGSPEDNEQKQLFVEQHKHYLDEIIEQISEFSKRVILADA |
| | | NLDKVLSAYNKHRDKPIREQAENIIHLFTLTNLGAPAAFKYFDTTIDRKRYT |
| | | STKEVLDATLIHQSITGLYETRIDLSQLGGDSGGSPKKKRKV |
| 24 | Cas9 ABE | MSEVEFSHEYWMRHALTLAKRARDEREVPVGAVLVLNNRVIGEGWNRAI |
| | ABE8.17m | GLHDPTAHAEIMALRQGGLVMQNYRLIDATLYSTFEPCVMCAGAMIHSRI |
| | [V106W] | GRVVFGWRNAKTGAAGSLMDVLHYPGMNHRVEITEGILADECAALLCYF |
| | | FRMPRRVFNAQKKAQSSTDSGGSSGGSSGSETPGTSESATPESSGGSSGGSD |
| | | KKYSIGLAIGTNSVGWAVITDEYKVPSKKFKVLGNTDRHSIKKNLIGALLFD |
| | | SGETAEATRLKRTARRRYTRRKNRICYLQEIFSNEMAKVDDSFFHRLEESFL |
| | | VEEDKKHERHPIFGNIVDEVAYHEKYPTIYHLRKKLVDSTDKADLRLIYLA |
| | | LAHMIKFRGHFLIEGDLNPDNSDVDKLFIQLVQTYNQLFEENPINASGVDA |
| | | KAILSARLSKSRRLENLIAQLPGEKKNGLFGNLIALSLGLTPNFKSNFDLAED |
| | | AKLQLSKDTYDDDLDNLLAQIGDQYADLFLAAKNLSDAILLSDILRVNTEIT |
| | | KAPLSASMIKRYDEHHQDLTLLKALVRQQLPEKYKEIFFDQSKNGYAGYID |
| | | GGASQEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAWMTRK |
| | | SEETITPWNFEEVVDKGASAQSFIERMTNFDKNLPNEKVLPKHSLLYEYFTV |
| | | YNELTKVKYVTEGMRKPAFLSGEQKKAIVDLLFKTNRKVTVKQLKEDYFK |
| | | KIECFDSVEISGVEDRFNASLGTYHDLLKIIKDKDFLDNEENEDILEDIVLTL |
| | | TLFEDREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSLHEHIA |
| | | NLAGSPAIKKGILQTVKVVDELVKVMGRHKPENIVIEMARENQTTQKGQK |
| | | NSRERMKRIEEGIKELGSQILKEHPVENTQLQNEKLYLYYLQNGRDMYVD |
| | | QELDINRLSDYDVDHIVPQSFLKDDSIDNKVLTRSDKNRGKSDNVPSEEVV |
| | | KKMKNYWRQLLNAKLITQRKFDNLTKAERGGLSELDKAGFIKRQLVETRQ |
| | | ITKHVAQILDSRMNTKYDENDKLIREVKVITLKSKLVSDFRKDFQFYKVREI |
| | | NNYHHAHDAYLNAVVGTALIKKYPKLESEFVYGDYKVYDVRKMIAKSEQ |
| | | EIGKATAKYFFYSNIMNFFKTEITLANGEIRKRPLIETNGETGEIVWDKGRDF |
| | | ATVRKVLSMPQVNIVKKTEVQTGGFSKESILPKRNSDKLIARKKDWDPKK |
| | | YGGFDSPTVAYSVLVVAKVEKGKSKKLKSVKELLGITIMERSSFEKNPIDFL |
| | | EAKGYKEVKKDLIIKLPKYSLFELENGRKRMLASAGELQKGNELALPSKYV |
| | | NFLYLASHYEKLKGSPEDNEQKQLFVEQHKHYLDEIIEQISEFSKRVILADA |
| | | NLDKVLSAYNKHRDKPIREQAENIIHLFTLTNLGAPAAFKYFDTTIDRKRYT |
| | | STKEVLDATLIHQSITGLYETRIDLSQLGGDEGADKRTADGSEFESPKKKRK |
| | | V |
| 25 | Cas13 ABE | MNIPALVENQKKYFGTYSVMAMLNAQTVLDHIQKVADIEGEQNENNENL |
| | REPAIRv1 | WFHPVMSHLYNAKNGYDKQPEKTMFIIERLQSYFPFLKIMAENQREYSNG |
| | | KYKQNRVEVNSNDIFEVLKRAFGVLKMYRDLTNAYKTYEEKLNDGCEFLT |
| | | STEQPLSGMINNYYTVALRNMNERYGYKTEDLAFIQDKRFKFVKDAYGKK |
| | | KSQVNTGFFLSLQDYNGDTQKKLHLSGVGIALLICLFLDKQYINIFLSRLPIF |
| | | SSYNAQSEERRIIIRSFGINSIKLPKDRIHSEKSNKSVAMDMLNEVKRCPDEL |
| | | FTTLSAEKQSRFRIISDDHNEVLMKRSSDRFVPLLLQYIDYGKLFDHIRFHV |
| | | NMGKLRYLLKADKTCIDGQTRVRVIEQPLNGFGRLEEAETMRKQENGTFG |
| | | NSGIRIRDFENMKRDDANPANYPYIVDTYTHYILENNKVEMFINDKEDSAP |
| | | LLPVIEDDRYVVKTIPSCRMSTLEIPAMAFHMFLFGSKKTEKLIVDVHNRYK |
| | | RLFQAMQKEEVTAENIASFGIAESDLPQKILDLISGNAHGKDVDAFIRLTVD |
| | | DMLTDTERRIKRFKDDRKSIRSADNKMGKRGFKQISTGKLADFLAKDIVLF |
| | | QPSVNDGENKITGLNYRIMQSAIAVYDSGDDYEAKQQFKLMFEKARLIGK |
| | | GTTEPHPFLYKVFARSIPANAVEFYERYLIERKFYLTGLSNEIKKGNRVDVP |
| | | FIRRDQNKWKTPAMKTLGRIYSEDLPVELPRQMFDNEIKSHLKSLPQMEGI |
| | | DFNNANVTYLIAEYMKRVLDDDFQTFYQWNRNYRYMDMLKGEYDRKGS |
| | | LQHCFTSVEEREGLWKERASRTERYRKQASNKIRSNRQMRNASSEEIETILD |
| | | KRLSNSRNEYQKSEKVIRRYRVQDALLFLLAKKTLTELADFDGERFKLKEI |
| | | MPDAEKGILSEIMPMSFTFEKGGKKYTITSEGMKLKNYGDFFVLASDKRIG |
| | | NLLELVGSDIVSKEDIMEEFNKYDQCRPEISSIVFNLEKWAFDTYPELSARV |
| | | DREEKVDFKSILKILLNNKNINKEQSDILRKIRNAFDANNYPDKGVVEIKAL |
| | | PEIAMSIKKAFGEYAIMKGSLQLPPLERLTLGSGGGGSQLHLPQVLADAVSR |
| | | LVLGKFGDLTDNFSSPHARRKVLAGVVMTTGTDVKDAKVISVSTGTKCIN |
| | | GEYMSDRGLALNDCHAEIISRRSLLRFLYTQLELYLNNKDDQKRSIFQKSER |
| | | GGFRLKENVQFHLYISTSPCGDARIFSPHEPILEEPADRHPNRKARGQLRTKI |
| | | ESGQGTIPVRSNASIQTWDGVLQGERLLTMSCSDKIARWNVVGIQGSLLSIF |
| | | VEPIYFSSIILGSLYHGDHLSRAMYQRISNIEDLPPLYTLNKPLLSGISNAEAR |
| | | QPGKAPNFSVNWTVGDSAIEVINATTGKDELGRASRLCKHALYCRWMRV |
| | | HGKVPSHLLRSKITKPNVYHESKLAAKEYQAAKARLFTAFIKAGLGAWVE |
| | | KPTEQDQFSLT |
| 26 | Cas13 ABE | MNIPALVENQKKYFGTYSVMAMLNAQTVLDHIQKVADIEGEQNENNENL |
| | REPAIRv2 | WFHPVMSHLYNAKNGYDKQPEKTMFIIERLQSYFPFLKIMAENQREYSNG |
| | | KYKQNRVEVNSNDIFEVLKRAFGVLKMYRDLTNAYKTYEEKLNDGCEFLT |
| | | STEQPLSGMINNYYTVALRNMNERYGYKTEDLAFIQDKRFKFVKDAYGKK |
| | | KSQVNTGFFLSLQDYNGDTQKKLHLSGVGIALLICLFLDKQYINIFLSRLPIF |
| | | SSYNAQSEERRIIIRSFGINSIKLPKDRIHSEKSNKSVAMDMLNEVKRCPDEL |
| | | FTTLSAEKQSRFRIISDDHNEVLMKRSSDRFVPLLLQYIDYGKLFDHIRFHV |
| | | NMGKLRYLLKADKTCIDGQTRVRVIEQPLNGFGRLEEAETMRKQENGTFG |
| | | NSGIRIRDFENMKRDDANPANYPYIVDTYTHYILENNKVEMFINDKEDSAP |
| | | LLPVIEDDRYVVKTIPSCRMSTLEIPAMAFHMFLFGSKKTEKLIVDVHNRYK |
| | | RLFQAMQKEEVTAENIASFGIAESDLPQKILDLISGNAHGKDVDAFIRLTVD |
| | | DMLTDTERRIKRFKDDRKSIRSADNKMGKRGFKQISTGKLADFLAKDIVLF |
| | | QPSVNDGENKITGLNYRIMQSAIAVYDSGDDYEAKQQFKLMFEKARLIGK |
| | | GTTEPHPFLYKVFARSIPANAVEFYERYLIERKFYLTGLSNEIKKGNRVDVP |
| | | FIRRDQNKWKTPAMKTLGRIYSEDLPVELPRQMFDNEIKSHLKSLPQMEGI |
| | | DFNNANVTYLIAEYMKRVLDDDFQTFYQWNRNYRYMDMLKGEYDRKGS |
| | | LQHCFTSVEEREGLWKERASRTERYRKQASNKIRSNRQMRNASSEEIETILD |
| | | KRLSNSRNEYQKSEKVIRRYRVQDALLFLLAKKTLTELADFDGERFKLKEI |
| | | MPDAEKGILSEIMPMSFTFEKGGKKYTITSEGMKLKNYGDFFVLASDKRIG |
| | | NLLELVGSDIVSKEDIMEEFNKYDQCRPEISSIVFNLEKWAFDTYPELSARV |
| | | DREEKVDFKSILKILLNNKNINKEQSDILRKIRNAFDANNYPDKGVVEIKAL |
| | | PEIAMSIKKAFGEYAIMKGSLQLPPLERLTLGSGGGGSQLHLPQVLADAVSR |
| | | LVLGKFGDLTDNFSSPHARRKVLAGVVMTTGTDVKDAKVISVSTGGKCIN |
| | | GEYMSDRGLALNDCHAEIISRRSLLRFLYTQLELYLNNKDDQKRSIFQKSER |
| | | GGFRLKENVQFHLYISTSPCGDARIFSPHEPILEEPADRHPNRKARGQLRTKI |
| | | ESGQGTIPVRSNASIQTWDGVLQGERLLTMSCSDKIARWNVVGIQGSLLSIF |
| | | VEPIYFSSIILGSLYHGDHLSRAMYQRISNIEDLPPLYTLNKPLLSGISNAEAR |
| | | QPGKAPNFSVNWTVGDSAIEVINATTGKDELGRASRLCKHALYCRWMRV |
| | | HGKVPSHLLRSKITKPNVYHESKLAAKEYQAAKARLFTAFIKAGLGAWVE |
| | | KPTEQDQFSLT |
| 27 | VanR | ATTGGATCCAAT |
| | operator | |
| 28 | TtgR | TATTTACAAACAACCATGAATGTAAGTA |
| | operator | |
| 29 | Gal4 UAS | CGGAGTACTGTCCTCCGA |
| | (for CID | |
| | systems) | |
| 30 | PhlF | ATGATACGAAACGTACCGTATCGTTAAGGT |
| | operator | |
| 31 | CymR | agaaacaaaccaacctgtctgtatta |
| | operator | |
| | v1 | |
| 32 | CymR | aacaaacagacaatctggtctgtttgta |
| | operator | |
| | v2 | |
| 33 | TetOff- | MSRLDKSKVINSALELLNEVGIEGLTTRKLAQKLGVEQPTLYWHVKNKRA |
| | Advanced | LLDALAIEMLDRHHTHFCPLEGESWQDFLRNNAKSFRCALLSHRDGAKVH |
| | | LGTRPTEKQYETLENQLAFLCQQGFSLENALYALSAVGHFTLGCVLEDQEH |
| | | QVAKEERETPTTDSMPPLLRQAIELFDHQGAEPAFLFGLELIICGLEKQLKCE |
| | | SGGPADALDDFDLDMLPADALDDFDLDMLPADALDDFDLDMLPG |
| 34 | VanR-VP16 | MDMPRIKPGQRVMMALRKMIASGEIKSGERIAEIPTAAALGVSRMPVRIAL |
| | | RSLEQEGLVVRLGARGYAARGVSSDQIRDAIEVRGVLEGFAARRLAERGM |
| | | TAETHARFVVLIAEGEALFAAGRLNGEDLDRYAAYNQAFHDTLVSAAGNG |
| | | AVESALARNGFEPFAAAGALALDLMDLSAEYEHLLAAHRQHQAVLDAVS |
| | | CGDAEGAERIMRDHALAAIRNAKVFEAAASAGAPLGAAWSIRADSGGGGP |
| | | TDALDDFDLDMLPADALDDFDLDMLPADALDDFDLDMLPGPPKKKRKV |
| 35 | TtgR-VP16 | MVRRTKEEAQETRAQIIEAAERAFYKRGVARTTLADIAELAGVTRGAIYW |
| | | HFNNKAELVQALLDSLHETHDHLARASESEDEVDPLGCMRKLLLQVFNEL |
| | | VLDARTRRINEILHHKCEFTDDMCEIRQQHQSAVLDCHKGITLTLANVVRR |
| | | GQLPGELDAERAAVAMFAYVDGLIRRWLLLPDSVDLLGDVEKWVDTGLD |
| | | MLRLSPALRKSGGGGPTDALDDFDLDMLPADALDDFDLDMLPADALDDF |
| | | DLDMLPGPPKKKRKV |
| 36 | PhlF-VP16 | MARTPSRSSIGSLRSPHTHKAILTSTIEILKECGYSGLSIESVARRAGAGKPTI |
| | | YRWWTNKAALIAEVYENEIEQVRKFPDLGSFKADLDFLLHNLWKVWRETI |
| | | CGEAFRCVIAEAQLDPVTLTQLKDQFMERRREIPKKLVEDAISNGELPKDIN |
| | | RELLLDMIFGFCWYRLLTEQLTVEQDIEEFTFLLINGVCPGTQCSGGGGPTD |
| | | ALDDFDLDMLPADALDDFDLDMLPADALDDFDLDMLPGPPKKKRKV |
| 37 | cTA | MSPKRRTQAERAMETQGKLIAAALGVLREKGYAGFRIADVPGAAGVSRGA |
| | | QSHHFPTKLELLLATFEWLYEQITERSRARLAKLKPEDDVIQQMLDDAAEF |
| | | FLDDDFSIGLDLIVAADRDPALREGIQRTVERNRFVVEDMWLGVLVSRGLS |
| | | RDDAEDILWLIFNSVRGLVVRSLWQKDKERFERVRNSTLEIARERYAKFKR |
| | | SGGGGPTDALDDFDLDMLPADALDDFDLDMLPADALDDFDLDMLPGPPK |
| | | KKRKV |
| 38 | Rep WT | MPGFYEIVIKVPSDLDEHLPGISDSFVNWVAEKEWELPPDSDMDLNLIEQAP |
| | | LTVAEKLQRDFLTEWRRVSKAPEALFFVQFEKGESYFHMHVLVETTGVKS |
| | | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYMELVGWLVDKGITSEKQWIQEDQASYISFN |
| | | AASNSRSQIKAALDNAGKIMSLTKTAPDYLVGQQPVEDISSNRIYKILELNG |
| | | YDPQYAASVFLGWATKKFGKRNTIWLFGPATTGKTNIAEAIAHTVPFYGC |
| | | VNWTNENFPFNDCVDKMVIWWEEGKMTAKVVESAKAILGGSKVRVDQK |
| | | CKSSAQIDPTPVIVTSNTNMCAVIDGNSTTFEHQQPLQDRMFKFELTRRLDH |
| | | DFGKVTKQEVKDFFRWAKDHVVEVEHEFYVKKGGAKKRPAPSDADISEP |
| | | KRVRESVAQPSTSDAEASINYADRYQNKCSRHVGMNLMLFPCRQCERMN |
| | | QNSNICFTHGQKDCLECFPVSESQPVSVVKKAYQKLCYIHHIMGKVPDACT |
| | | ACDLVNVDLDDCIFEQ |
| 39 | L4 100K | MESVEKEDSLTAPFEFATTASTDAANAPTTFPVEAPPLEEEEVIIEQDPGFVSE |
| | (wt) | DDEDRSVPTEDKKQDQDDAEANEEQVGRGDQRHGDYLDVGDDVLLKHLQ |
| | | RQCAIICDALQERSDVPLAIADVSLAYERHLFSPRVPPKRQENGTCEPNPRLN |
| | | FYPVFAVPEVLATYHIFFQNCKIPLSCRANRSRADKQLALRQGAVIPDIASLD |
| | | EVPKIFEGLGRDEKRAANALQQENSENESHCGVLVELEGDNARLAVLKRSIE |
| | | VTHFAYPALNLPPKVMSTVMSELIVRRARPLERDANLQEQTEEGLPAVGDEQ |
| | | LARWLETREPADLEERRKLMMAAVLVTVELECMQRFFADPEMQRKLEETL |
| | | HYTFRQGYVRQACKISNVELCNLVSYLGILHENRLGQNVLHSTLKGEARRD |
| | | YVRDCVYLFLCYTWQTAMGVWQQCLEERNLKELQKLLKQNLKDLWTAFN |
| | | ERSVAAHLADIIFPERLLKTLQQGLPDFTSQSMLQNFRNFILERSGILPATCCA |
| | | LPSDFVPIKYRECPPPLWGHCYLLQLANYLAYHSDIMEDVSGDGLLECHCRC |
| | | NLCTPHRSLVCNSQLLSESQIIGTFELQGPSPDEKSAAPGLKLTPGLWTSAYLR |
| | | KFVPEDYHAHEIRFYEDQSRPPNAELTACVITQGHILGQLQAINKARQEFLLR |
| | | KGRGVYLDPQSGEELNPIPPPPQPYQQPRALASQDGTQKEAAAAAAATHGR |
| | | GGILGQSGRGGFGRGGGDDGRLGQPRRSFRGRRGVRRNTVTLGRIPLAGAPE |
| | | IGNRSQHRYNLRSSGAAGTACSPTQP |
| 40 | VP (wt) | ATGGCTGCCGATGGTTATCTTCCAGATTGGCTCGAGGACACTCTCTCTGA |
| | | AGGAATAAGACAGTGGTGGAAGCTCAAACCTGGCCCACCACCACCAAAG |
| | | CCCGCAGAGCGGCATAAGGACGACAGCAGGGGTCTTGTGCTTCCTGGGT |
| | | ACAAGTACCTCGGACCCTTCAACGGACTCGACAAGGGAGAGCCGGTCAA |
| | | CGAGGCAGACGCCGCGGCCCTCGAGCACGACAAAGCCTACGACCGGCAG |
| | | CTCGACAGCGGAGACAACCCGTACCTCAAGTACAACCACGCCGACGCGG |
| | | AGTTTCAGGAGCGCCTTAAAGAAGATACGTCTTTTGGGGGCAACCTCGGA |
| | | CGAGCAGTCTTCCAGGCGAAAAAGAGGGTTCTTGAACCTCTGGGCCTGGT |
| | | TGAGGAACCTGTTAAGACGGCTCCGGGAAAAAAGAGGCCGGTAGAGCAC |
| | | TCTCCTGTGGAGCCAGACTCCTCCTCGGGAACCGGAAAGGCGGGCCAGC |
| | | AGCCTGCAAGAAAAAGATTGAATTTTGGTCAGACTGGAGACGCAGACTC |
| | | AGTACCTGACCCCCAGCCTCTCGGACAGCCACCAGCAGCCCCCTCTGGTC |
| | | TGGGAACTAATACGATGGCTACAGGCAGTGGCGCACCAATGGCAGACAA |
| | | TAACGAGGGCGCCGACGGAGTGGGTAATTCCTCGGGAAATTGGCATTGC |
| | | GATTCCACATGGATGGGCGACAGAGTCATCACCACCAGCACCCGAACCT |
| | | GGGCCCTGCCCACCTACAACAACCACCTCTACAAACAAATTTCCAGCCAA |
| | | TCAGGAGCCTCGAACGACAATCACTACTTTGGCTACAGCACCCCTTGGGG |
| | | GTATTTTGACTTCAACAGATTCCACTGCCACTTTTCACCACGTGACTGGCA |
| | | AAGACTCATCAACAACAACTGGGGATTCCGACCCAAGAGACTCAACTTC |
| | | AAGCTCTTTAACATTCAAGTCAAAGAGGTCACGCAGAATGACGGTACGA |
| | | CGACGATTGCCAATAACCTTACCAGCACGGTTCAGGTGTTTACTGACTCG |
| | | GAGTACCAGCTCCCGTACGTCCTCGGCTCGGCGCATCAAGGATGCCTCCC |
| | | GCCGTTCCCAGCAGACGTCTTCATGGTGCCACAGTATGGATACCTCACCC |
| | | TGAACAACGGGAGTCAGGCAGTAGGACGCTCTTCATTTTACTGCCTGGAG |
| | | TACTTTCCTTCTCAGATGCTGCGTACCGGAAACAACTTTACCTTCAGCTAC |
| | | ACTTTTGAGGACGTTCCTTTCCACAGCAGCTACGCTCACAGCCAGAGTCT |
| | | GGACCGTCTCATGAATCCTCTCATCGACCAGTACCTGTATTACTTGAGCA |
| | | GAACAAACACTCCAAGTGGAACCACCACGCAGTCAAGGCTTCAGTTTTCT |
| | | CAGGCCGGAGCGAGTGACATTCGGGACCAGTCTAGGAACTGGCTTCCTG |
| | | GACCCTGTTACCGCCAGCAGCGAGTATCAAAGACATCTGCGGATAACAA |
| | | CAACAGTGAATACTCGTGGACTGGAGCTACCAAGTACCACCTCAATGGCA |
| | | GAGACTCTCTGGTGAATCCGGGCCCGGCCATGGCAAGCCACAAGGACGA |
| | | TGAAGAAAAGTTTTTTCCTCAGAGCGGGGTTCTCATCTTTGGGAAGCAAG |
| | | GCTCAGAGAAAACAAATGTGGACATTGAAAAGGTCATGATTACAGACGA |
| | | AGAGGAAATCAGGACAACCAATCCCGTGGCTACGGAGCAGTATGGTTCT |
| | | GTATCTACCAACCTCCAGAGAGGCAACAGACAAGCAGCTACCGCAGATG |
| | | TCAACACACAAGGCGTTCTTCCAGGCATGGTCTGGCAGGACAGAGATGTG |
| | | TACCTTCAGGGGCCCATCTGGGCAAAGATTCCACACACGGACGGACATTT |
| | | TCACCCCTCTCCCCTCATGGGTGGATTCGGACTTAAACACCCTCCTCCACA |
| | | GATTCTCATCAAGAACACCCCGGTACCTGCGAATCCTTCGACCACCTTCA |
| | | GTGCGGCAAAGTTTGCTTCCTTCATCACACAGTACTCCACGGGACAGGTC |
| | | AGCGTGGAGATCGAGTGGGAGCTGCAGAAGGAAAACAGCAAACGCTGG |
| | | AATCCCGAAATTCAGTACACTTCCAACTACAACAAGTCTGTTAATGTGGA |
| | | CTTTACTGTGGACACTAATGGCGTGTATTCAGAGCCTCGCCCCATTGGCA |
| | | CCAGATACCTGACTCGTAATCTGTAA |
| 41 | VP (wt) | MAADGYLPDWLEDTLSEGIRQWWKLKPGPPPPKPAERHKDDSRGLVLPGYK |
| | Translated | YLGPFNGLDKGEPVNEADAAALEHDKAYDRQLDSGDNPYLKYNHADAEFQ |
| | (same as | ERLKEDTSFGGNLGRAVFQAKKRVLEPLGLVEEPVKTAPGKKRPVEHSPVE |
| | SEQ | PDSSSGTGKAGQQPARKRLNFGQTGDADSVPDPQPLGQPPAAPSGLGTN |
| | ID NO: 14 | TMATGSGAPMADNNEGADGVGNSSGNWHCDSTWMGDRVITTSTRTWALPTYNN |
| | with | HLYKQISSQSGASNDNHYFGYSTPWGYFDFNRFHCHFSPRDWQRLINNNWGFRP |
| | different | KRLNFKLFNIQVKEVTQNDGTTTIANNLTSTVQVFTDSEYQLPYVLGSAHQGCLPP |
| | VP protein | FPADVFMVPQYGYLTLNNGSQAVGRSSFYCLEYFPSQMLRTGNNFTFSYTFEDVP |
| | identified) | FHSSYAHSQSLDRLMNPLIDQYLYYLSRTNTPSGTTTQSRLQFSQAGASDIRDQSRN |
| | Underline: | WLPGPCYRQQRVSKTSADNNNSEYSWTGATKYHLNGRDSLVNPGPAMASHKDDE |
| | VP1 | EKFFPQSGVLIFGKQGSEKTNVDIEKVMITDEEEIRTTNPVATEQYGSVSTNLQRG |
| | Bold: VP2 | NRQAATADVNTQGVLPGMVWQDRDVYLQGPIWAKIPHTDGHFHPSPLMGGFGL |
| | Italic: VP3 | KHPPPQILIKNTPVPANPSTTFSAAKFASFITQYSTGQVSVEIEWELQKENSKRWNP |
| | | EIQYTSNYNKSVNVDFTVDTNGVYSEPRPIGTRYLTRNL |
| 42 | rcTA | MVIMSPKRRTQAERAMETQGKLIAAALGVLREKGYAGFRIADVPGAAGVSR |
| | (example | GAQSHHFPTKLELLLATFEWLYEQITERSRARLAKLKPEDDVIQQMLDDAAE |
| | sequence | FFLDDDFSIGLDLIVAADRDPVLREGIQRTVERNRFVVGDIWLGVLVSRGLSR |
| | with | DDAEDILWLIFNSVRGLVVRSLWQKDKERFERVRNSTLEIARERYAKFKRSG |
| | reverse | GGGPTDALDDFDLDMLPADALDDFDLDMLPADALDDFDLDMLPGPPKKKR |
| | CymR fused | KV |
| | to 3x | |
| | VP16 and | |
| | a NLS) | |
| 43 | MAAP (wt) | LAHHHQSPQSGIRTTAGVLCFLGTSTSDPSTDSTRESRSTRQTPRPSSTTKPTT |
| | | GSSTAETTRTSSTTTPTRSFRSALKKIRLLGATSDEQSSRRKRGFLNLWAWLR |
| | | NLLRRLREKRGR |
| 44 | AncBE4max | MKRTADGSEFESPKKKRKVSSETGPVAVDPTLRRRIEPHEFEVFFDPRELRKE |
| | | TCLLYEIKWGTSHKIWRHSSKNTTKHVEVNFIEKFTSERHFCPSTSCSITWFLS |
| | | WSPCGECSKAITEFLSQHPNVTLVIYVARLYHHMDQQNRQGLRDLVNSGVTI |
| | | QIMTAPEYDYCWRNFVNYPPGKEAHWPRYPPLWMKLYALELHAGILGLPPC |
| | | LNILRRKQPQLTFFTIALQSCHYQRLPPHILWATGLKSGGSSGGSSGSETPGTS |
| | | ESATPESSGGSSGGSDKKYSIGLAIGTNSVGWAVITDEYKVPSKKFKVLGNTD |
| | | RHSIKKNLIGALLFDSGETAEATRLKRTARRRYTRRKNRICYLQEIFSNEMAK |
| | | VDDSFFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYNQLFEE |
| | | NPINASGVDAKAILSARLSKSRRLENLIAQLPGEKKNGLFGNLIALSLGLTPNF |
| | | KSNFDLAEDAKLQLSKDTYDDDLDNLLAQIGDQYADLFLAAKNLSDAILLSD |
| | | ILRVNTEITKAPLSASMIKRYDEHHQDLTLLKALVRQQLPEKYKEIFFDQSKN |
| | | GYAGYIDGGASQEEFYKFIKPILEKMDGTEELLVKLNREDLLRKQRTFDNGSI |
| | | PHQIHLGELHAILRRQEDFYPFLKDNREKIEKILTFRIPYYVGPLARGNSRFAW |
| | | MTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKED |
| | | YFKKIECFDSVEISGVEDRFNASLGTYHDLLKIIKDKDFLDNEENEDILEDIVL |
| | | TLTLFEDREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSLHEHIAN |
| | | LAGSPAIKKGILQTVKVVDELVKVMGRHKPENIVIEMARENQTTQKGQKNS |
| | | RERMKRIEEGIKELGSQILKEHPVENTQLQNEKLYLYYLQNGRDMYVDQEL |
| | | DINRLSDYDVDHIVPQSFLKDDSIDNKVLTRSDKNRGKSDNVPSEEVVKKMK |
| | | NYWRQLLNAKLITQRKFDNLTKAERGGLSELDKAGFIKRQLVETRQITKHVA |
| | | QILDSRMNTKYDENDKLIREVKVITLKSKLVSDFRKDFQFYKVREINNYHHA |
| | | HDAYLNAVVGTALIKKYPKLESEFVYGDYKVYDVRKMIAKSEQEIGKATAK |
| | | YFFYSNIMNFFKTEITLANGEIRKRPLIETNGETGEIVWDKGRDFATVRKVLS |
| | | MPQVNIVKKTEVQTGGFSKESILPKRNSDKLIARKKDWDPKKYGGFDSPTVA |
| | | YSVLVVAKVEKGKSKKLKSVKELLGITIMERSSFEKNPIDFLEAKGYKEVKK |
| | | DLIIKLPKYSLFELENGRKRMLASAGELQKGNELALPSKYVNFLYLASHYEK |
| | | LKGSPEDNEQKQLFVEQHKHYLDEIIEQISEFSKRVILADANLDKVLSAYNKH |
| | | RDKPIREQAENIIHLFTLTNLGAPAAFKYFDTTIDRKRYTSTKEVLDATLIHQSI |
| | | TGLYETRIDLSQLGGDSGGSGGSGGSTNLSDIIEKETGKQLVIQESILMLPEEV |
| | | EEVIGNKPESDILVHTAYDESTDENVMLLTSDAPEYKPWALVIQDSNGENKI |
| | | KMLSGGSGGSGGSTNLSDIIEKETGKQLVIQESILMLPEEVEEVIGNKPESDIL |
| | | VHTAYDESTDENVMLLTSDAPEYKPWALVIQDSNGENKIKMLSGGSKRTAD |
| | | GSEFEPKKKRKV |
| 45 | hyBE4max | MKRTADGSEFESPKKKRKVSSETGPVAVDPTLRRRIEPHEFEVFFDPRELRKE |
| | | TCLLYEINWGGRHSIWRHTSQNTNKHVEVNFIEKFTTERYFCPNTRCSITWFL |
| | | SWSPCGECSRAITEFLSRYPHVTLFIYIARLYHHADPRNRQGLRDLISSGVTIQI |
| | | MTEQESGYCWRNFVNYSPSNEAHWPRYPHLWVRLYVLELYCIILGLPPCLNI |
| | | LRRKQPQLTFFTIALQSCHYQRLPPHILWATGLKSGGSSGGSSGSETPGTSESA |
| | | TPESSGGSSGGSMAMQMQLEANADTSVEEESFGPQPISRLEQCGINANDVKK |
| | | LEEAGFHTVEAVAYAPKKELINIKGISEAKADKILAEAAKLVPMGFTTATEFH |
| | | QRRSEIIQITTGSKELDKLLQSGGSSGGSSGSETPGTSESATPESSGGSSGGSDK |
| | | KYSIGLAIGTNSVGWAVITDEYKVPSKKFKVLGNTDRHSIKKNLIGALLFDSG |
| | | ETAEATRLKRTARRRYTRRKNRICYLQEIFSNEMAKVDDSFFHRLEESFLVEE |
| | | DKKHERHPIFGNIVDEVAYHEKYPTIYHLRKKLVDSTDKADLRLIYLALAHM |
| | | IKFRGHFLIEGDLNPDNSDVDKLFIQLVQTYNQLFEENPINASGVDAKAILSAR |
| | | LSKSRRLENLIAQLPGEKKNGLFGNLIALSLGLTPNFKSNFDLAEDAKLQLSK |
| | | DTYDDDLDNLLAQIGDQYADLFLAAKNLSDAILLSDILRVNTEITKAPLSASM |
| | | IKRYDEHHQDLTLLKALVRQQLPEKYKEIFFDQSKNGYAGYIDGGASQEEFY |
| | | KFIKPILEKMDGTEELLVKLNREDLLRKQRTFDNGSIPHQIHLGELHAILRRQE |
| | | DFYPFLKDNREKIEKILTFRIPYYVGPLARGNSRFAWMTRKSEETITPWNFEE |
| | | VVDKGASAQSFIERMTNFDKNLPNEKVLPKHSLLYEYFTVYNELTKVKYVT |
| | | EGMRKPAFLSGEQKKAIVDLLFKTNRKVTVKQLKEDYFKKIECFDSVEISGV |
| | | EDRFNASLGTYHDLLKIIKDKDFLDNEENEDILEDIVLTLTLFEDREMIEERLK |
| | | TYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTV |
| | | KVVDELVKVMGRHKPENIVIEMARENQTTQKGQKNSRERMKRIEEGIKELG |
| | | SQILKEHPVENTQLQNEKLYLYYLQNGRDMYVDQELDINRLSDYDVDHIVP |
| | | QSFLKDDSIDNKVLTRSDKNRGKSDNVPSEEVVKKMKNYWRQLLNAKLITQ |
| | | RKFDNLTKAERGGLSELDKAGFIKRQLVETRQITKHVAQILDSRMNTKYDEN |
| | | DKLIREVKVITLKSKLVSDFRKDFQFYKVREINNYHHAHDAYLNAVVGTALI |
| | | KKYPKLESEFVYGDYKVYDVRKMIAKSEQEIGKATAKYFFYSNIMNFFKTEI |
| | | TLANGEIRKRPLIETNGETGEIVWDKGRDFATVRKVLSMPQVNIVKKTEVQT |
| | | GGFSKESILPKRNSDKLIARKKDWDPKKYGGFDSPTVAYSVLVVAKVEKGK |
| | | SKKLKSVKELLGITIMERSSFEKNPIDFLEAKGYKEVKKDLIIKLPKYSLFELE |
| | | NGRKRMLASAGELQKGNELALPSKYVNFLYLASHYEKLKGSPEDNEQKQLF |
| | | VEQHKHYLDEIIEQISEFSKRVILADANLDKVLSAYNKHRDKPIREQAENIIHL |
| | | FTLTNLGAPAAFKYFDTTIDRKRYTSTKEVLDATLIHQSITGLYETRIDLSQLG |
| | | GDSGGSGGSGGSTNLSDIIEKETGKQLVIQESILMLPEEVEEVIGNKPESDILV |
| | | HTAYDESTDENVMLLTSDAPEYKPWALVIQDSNGENKIKMLSGGSGGSGGS |
| | | TNLSDIIEKETGKQLVIQESILMLPEEVEEVIGNKPESDILVHTAYDESTDENV |
| | | MLLTSDAPEYKPWALVIQDSNGENKIKMLSGGSKRTADGSEFEPKKKRKV |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. An engineered cell for AAV production, comprising one or more stably integrated nucleic acid molecules collectively comprising a nucleic acid sequence encoding for each of: Rep52, DA-Rep52, Rep40, or DA-Rep40; Rep78, DA-Rep78, Rep68, or DA-Rep68; E2A or DA-E2A; E4ORF6 or DA-E4ORF6; VARNA or DA-VARNA; VP1 or DA-VP1; VP2 or DA-VP2; VP3 or DA-VP3; AAP or DA-AAP; MAAP or DA-MAAP; L4 100K or DA-L4 100K; and a Base Editor, each nucleic acid molecule being operably linked to a promoter; wherein the cell comprises the nucleic acid sequence of at least one of DA-Rep52, DA-Rep40, DA-Rep78, DA-Rep68, DA-E2A, DA-E4ORF6, DA-VP1, DA-VP2, DA-VP3, DA-AAP, DA-MAAP, and DA-L4 100K; wherein the nucleic acid sequences of DA-Rep52, DA-Rep40, DA-Rep78, DA-Rep68, DA-E2A, DA-E4ORF6, DA-VP1, DA-VP2, DA-VP3, DA-AAP, DA-MAAP, and DA-L4 100K each comprises a mutated start codon.

2. The engineered cell of claim 1, wherein the AAV production system comprises a nucleic acid sequence encoding for DA-Rep52, wherein the DA-Rep52 has at least 80% identity with SEQ ID NO: 6, and wherein the amino acid at position 1 of DA-Rep52 is:
(i) an isoleucine residue encoded by an ATA codon;
(ii) a threonine residue encoded by an ACG codon;
(iii) a valine residue encoded by a GTG codon; or
(iv) an alanine codon encoded by a GCG codon.

3. The engineered cell of claim 2, wherein the nucleic acid sequence encoding for DA-Rep52 further comprises one or more non-synonymous codons.

4. The engineered cell of claim 2 or claim 3, wherein the nucleic acid sequence encoding for DA-Rep52 further comprises one or more premature stop codons.

5. The engineered cell of any one of claims 1-4, wherein the AAV production system comprises a nucleic acid sequence encoding for DA-Rep40, wherein the DA-Rep40 has at least 80% identity with SEQ ID NO: 7, and wherein the amino acid at position 1 of DA-Rep40 is:
(i) an isoleucine residue encoded by an ATA codon;
(ii) a threonine residue encoded by an ACG codon;
(iii) a valine residue encoded by a GTG codon; or
(iv) an alanine codon encoded by a GCG codon.

6. The engineered cell of claim 5, wherein the nucleic acid sequence encoding for DA-Rep40 further comprises one or more non-synonymous codons.

7. The engineered cell of claim 5 or claim 6, wherein the nucleic acid sequence encoding for DA-Rep40 further comprises one or more premature stop codons.

8. The engineered cell of any one of claims 1-7, wherein the AAV production system comprises a nucleic acid sequence encoding for DA-Rep78, wherein the DA-Rep78 has at least 80% identity with SEQ ID NO: 8, and wherein the amino acid at position 1 of DA-Rep78 is:
(i) an isoleucine residue encoded by an ATA codon;
(ii) a threonine residue encoded by an ACG codon;
(iii) a valine residue encoded by a GTG codon; or
(iv) an alanine codon encoded by a GCG codon.

9. The engineered cell of claim 8, wherein the nucleic acid sequence encoding for DA-Rep78 further comprises one or more non-synonymous codons.

10. The engineered cell of claim 8 or claim 9, wherein the nucleic acid sequence encoding for DA-Rep78 further comprises one or more premature stop codons.

11. The engineered cell of any one of claims 1-10, wherein the AAV production system comprises a nucleic acid sequence encoding for DA-Rep68, wherein the DA-Rep68 has at least 80% identity with SEQ ID NO: 9, and wherein the amino acid at position 1 of DA-Rep68 is:
(i) an isoleucine residue encoded by an ATA codon;
(ii) a threonine residue encoded by an ACG codon;
(iii) a valine residue encoded by a GTG codon; or
(iv) an alanine codon encoded by a GCG codon.

12. The engineered cell of claim 11, wherein the nucleic acid sequence encoding for DA-Rep68 further comprises one or more non-synonymous codons.

13. The engineered cell of claim 11 or claim 12, wherein the nucleic acid sequence encoding for DA-Rep68 further comprises one or more premature stop codons.

14. The engineered cell of any one of claims 1-13, wherein the AAV production system comprises a nucleic acid sequence encoding for DA-E2A, wherein the DA-E2A has at least 80% identity with SEQ ID NO: 10, and wherein the amino acid at position 1 of DA-E2A is:
(i) an isoleucine residue encoded by an ATA codon;
(ii) a threonine residue encoded by an ACG codon;
(iii) a valine residue encoded by a GTG codon; or
(iv) an alanine codon encoded by a GCG codon.

15. The engineered cell of claim 14, wherein the nucleic acid sequence encoding for DA-E2A further comprises one or more non-synonymous codons.

16. The engineered cell of claim 14 or claim 15, wherein the nucleic acid sequence encoding for DA-E2A further comprises one or more premature stop codons.

17. The engineered cell of any one of claims 1-16, wherein the AAV production system comprises a nucleic acid sequence encoding for DA-E4ORF6, wherein the DA-E4ORF6 has at least 80% identity with SEQ ID NO: 11 or SEQ ID NO: 12, and wherein the amino acid at position 1 of DA-E4ORF6 is:
(i) an isoleucine residue encoded by an ATA codon;
(ii) a threonine residue encoded by an ACG codon;
(iii) a valine residue encoded by a GTG codon; or
(iv) an alanine codon encoded by a GCG codon.

18. The engineered cell of claim 17, wherein the nucleic acid sequence encoding for DA-E4ORF6 further comprises one or more non-synonymous codons.

19. The engineered cell of claim 17 or claim 18, wherein the nucleic acid sequence encoding for DA-E4ORF6 further comprises one or more premature stop codons.

20. The engineered cell of any one of claims 1-19, wherein the AAV production system comprises a nucleic acid sequence encoding for DA-VP1, wherein the DA-VP1 has at least 80% identity with SEQ ID NO: 14, and wherein the amino acid at position 1 of DA-VP1 is:
(i) an isoleucine residue encoded by an ATA codon;
(ii) a threonine residue encoded by an ACG codon;
(iii) a valine residue encoded by a GTG codon; or
(iv) an alanine codon encoded by a GCG codon.

21. The engineered cell of claim 20, wherein the nucleic acid sequence encoding for DA-VP1 further comprises one or more non-synonymous codons.

22. The engineered cell of claim 20 or claim 21, wherein the nucleic acid sequence encoding for DA-VP1 further comprises one or more premature stop codons.

23. The engineered cell of any one of claims 1-22, wherein the AAV production system comprises a nucleic acid sequence encoding for DA-VP2, wherein the DA-VP2 has at least 80% identity with SEQ ID NO: 15, and wherein the amino acid at position 1 of DA-VP2 is:
(i) a threonine residue encoded by an ACA codon; or
(ii) an alanine codon encoded by a GCG codon.

24. The engineered cell of claim 23, wherein the nucleic acid sequence encoding for DA-VP2 further comprises one or more non-synonymous codons.

25. The engineered cell of claim 23 or claim 24, wherein the nucleic acid sequence encoding for DA-VP2 further comprises one or more premature stop codons.

26. The engineered cell of any one of claims 1-25, wherein the AAV production system comprises a nucleic acid sequence encoding for DA-VP3, wherein the DA-VP3 has at least 80% identity with SEQ ID NO: 16, and wherein the amino acid at position 1 of DA-VP3 is:
(i) an isoleucine residue encoded by an ATA codon;
(ii) a threonine residue encoded by an ACG codon;
(iii) a valine residue encoded by a GTG codon; or
(iv) an alanine codon encoded by a GCG codon.

27. The engineered cell of claim 26, wherein the nucleic acid sequence encoding for DA-VP3 further comprises one or more non-synonymous codons.

28. The engineered cell of claim 26 or claim 27, wherein the nucleic acid sequence encoding for DA-VP3 further comprises one or more premature stop codons.

29. The engineered cell of any one of claims 1-28, wherein the AAV production system comprises a nucleic acid sequence encoding for DA-AAP, wherein the DA-AAP has at least 80% identity with SEQ ID NO: 17, and wherein the amino acid at position 1 of DA-AAP is:
(i) a leucine residue encoded by an CTA codon;
(ii) a leucine residue encoded by an TTG codon; or
(iii) a proline residue encoded by a CCG codon.

30. The engineered cell of claim 29, wherein the nucleic acid sequence encoding for DA-AAP further comprises one or more non-synonymous codons.

31. The engineered cell of claim 29 or claim 30, wherein the nucleic acid sequence encoding for DA-AAP further comprises one or more premature stop codons.

32. The engineered cell of any one of claims 1-31, wherein the AAV production system comprises a nucleic acid sequence encoding for DA-MAAP, wherein the DA-MAAP has at least 80% identity with SEQ ID NO: 43, and wherein the amino acid at position 1 of DA-MAAP is:
(i) a leucine residue encoded by an CTA codon;
(ii) a leucine residue encoded by an TTG codon; or
(iii) a proline residue encoded by a CCG codon.

33. The engineered cell of claim 32, wherein the nucleic acid sequence encoding for DA-MAAP further comprises one or more non-synonymous codons.

34. The engineered cell of claim 32 or claim 33, wherein the nucleic acid sequence encoding for DA-MAAP further comprises one or more premature stop codons.

35. The engineered cell of any one of claims 1-34, wherein the AAV production system comprises a nucleic acid sequence encoding for DA-L4 100K, wherein the DA-L4 100K has at least 80% identity with SEQ ID NO: 39, and wherein the amino acid at position 1 of DA-L4 100K is:
(i) an isoleucine residue encoded by an ATA codon;
(ii) a threonine residue encoded by an ACG codon;
(iii) a valine residue encoded by a GTG codon; or
(iv) an alanine codon encoded by a GCG codon.

36. The engineered cell of claim 35, wherein the nucleic acid sequence encoding for DA-L4 100K further comprises one or more non-synonymous codons.

37. The engineered cell of claim 35 or claim 36, wherein the nucleic acid sequence encoding for DA-L4 100K further comprises one or more premature stop codons.

38. The engineered cell of any one of claims 1-37, wherein the AAV production system comprises a nucleic acid sequence encoding for DA-VARNA, wherein the DA-VARNA comprises one or more non-synonymous codons and has at least 80% identity with SEQ ID NO: 13.

39. The engineered cell of claim 38, wherein the nucleic acid sequence encoding for DA-VARNA further comprises one or more premature terminators.

40. The engineered cell of any one of claims 1-39, wherein an inducible promoter is operably linked to the nucleic acid sequence encoding for one or more of: Rep52, DA-Rep52, Rep40, or DA-Rep40; Rep78, DA-Rep78, Rep68, or DA-Rep68; E2A or DA-E2A; E4ORF6 or DA-E4ORF6; VARNA or DA-VARNA; VP1 or DA-VP1; VP2 or DA-VP2; VP3 or DA-VP3; AAP or DA-AAP; MAAP or DA-MAAP; and L4 100K or DA-L4 100K.

41. The engineered cell of any one of claims 1-40, wherein an inducible promoter is operably linked to the nucleic acid sequence encoding for the Base Editor.

42. The engineered cell of claim 40 or claim 41, further comprising a nucleic acid sequence encoding for a transcriptional activator that is capable of binding to the inducible promoter.

43. The engineered cell of claim 42, wherein the transcriptional activator is selected from the group consisting of TetOn-3G, TetOn-V16, TetOff-Advanced, VanR-VP16, TtgR-VP16, PhlF-VP16, and the cumate cTA and rcTA.

44. The engineered cell of claim 43, wherein the transcriptional activator is TetOn 3G.

45. The engineered cell of any one of claims 1-44, wherein the Base Editor comprises an Adenine Base Editor (ABE).

46. The engineered cell of claim 45, wherein the ABE is Cas9-ABE or Cas13-ABE.

47. The engineered cell of claim 46, wherein the Cas9-ABE is encoded for by an amino acid sequence having at least 80% identity with SEQ ID NO: 23 or 24.

48. The engineered cell of claim 46, wherein the Cas13-ABE is encoded for by an amino acid sequence having at least 80% identity with SEQ ID NO: 25 or 26.

49. The engineered cell of any one of claims 1-48, wherein the Base Editor comprises a Cytosine Base Editor (CBE).

50. The engineered cell of claim 49, wherein the CBE is Cas9-CBE or Cas13-CBE.

51. The engineered cell of claim 50, wherein the Cas9-CBE is encoded for by an amino acid sequence having at least 80% identity with SEQ ID NO: 44 or 45.

52. The engineered cell of any one of claims 1-51, wherein the engineered cell is a HEK293 or HeLa cell.

53. A kit comprising the engineered cell of any one of claims 1-52.

54. The kit of claim 53 further comprising a polynucleotide comprising, from 5′ to 3′: (i) a nucleic acid sequence of a 5′ inverted terminal repeat; (ii) a multiple cloning site; and (iii) a nucleic acid sequence of a 3′ inverted terminal repeat.

55. The kit of claim 54, wherein the polynucleotide is a plasmid or a vector.

56. A method for AAV production, comprising contacting the engineered cell of any one of claims 42-44 with a small molecule inducer that binds to the transcriptional activator.

57. The method of claim 56, wherein the small molecule inducer is selected from the group consisting of doxycycline, vanillate, phloretin, rapamycin, abscisic acid, gibberellic acid acetoxymethyl ester, and cumate.

58. A method for AAV production, comprising expressing in the engineered cell of any one of claims 1-52 one or more single guide RNA(s) capable of targeting the Base Editor to a mutated start codon of DA-Rep52, DA-Rep40, DA-Rep78, DA-Rep68, DA-E2A, DA-E4ORF6, DA-VP1, DA-VP2, DA-VP3, DA-AAP, DA-MAAP, or DA-L4 100K, wherein the engineered cell comprising one or more polynucleotides encoding the one or more single guide RNA(s).

59. The method of claim 58, wherein one or more of DA-Rep52, DA-Rep40, DA-Rep78, DA-Rep68, DA-E2A, DA-E4ORF6, DA-VP1, DA-VP2, DA-VP3, DA-AAP, DA-MAAP, and DA-L4 100K further comprises a premature stop codon, and wherein the method further comprises expressing in the engineered cell one or more single guide RNA(s) capable of targeting the Base Editor to the premature stop codon of DA-Rep52, DA-Rep40, DA-Rep78, DA-Rep68, DA-E2A, DA-E4ORF6, DA-VP1, DA-VP2, DA-VP3, DA-AAP, DA-MAAP, and/or DA-L4 100K.

60. The method of claim 58 or claim 59, wherein one or more of DA-Rep52, DA-Rep40, DA-Rep78, DA-Rep68, DA-E2A, DA-E4ORF6, DA-VP1, DA-VP2, DA-VP3, DA-AAP, DA-MAAP, and DA-L4 100K further comprises a non-synonymous codon, and wherein the method further comprises expressing in the engineered cell one or more single guide RNA(s) capable of targeting the Base Editor to the non-synonymous codon of DA-Rep52, DA-Rep40, DA-Rep78, DA-Rep68, DA-E2A, DA-E4ORF6, DA-VP1, DA-VP2, DA-VP3, DA-AAP, DA-MAAP, and/or DA-L4 100K.

61. The method of any one of claims 58-60, wherein the one or more polynucleotides encoding the one or more single guide RNA(s) are stably integrated.

62. A method for AAV production, comprising contacting the engineered cell of any one of claims 1-52 with one or more single guide RNA(s) capable of targeting the Base Editor to a mutated start codon of DA-Rep52, DA-Rep40, DA-Rep78, DA-Rep68, DA-E2A, DA-E4ORF6, DA-VP1, DA-VP2, DA-VP3, DA-AAP, DA-MAAP, or DA-L4 100K such that that engineered cell internalizes the one or more single guide RNA(s).

63. The method of claim 62, wherein one or more of DA-Rep52, DA-Rep40, DA-Rep78, DA-Rep68, DA-E2A, DA-E4ORF6, DA-VP1, DA-VP2, DA-VP3, DA-AAP, DA-MAAP, and DA-L4 100K further comprises a premature stop codon, and wherein the method further comprises contacting the engineered cell with one or more single guide RNA(s) capable of targeting the Base Editor to the premature stop codon of DA-Rep52, DA-Rep40, DA-Rep78, DA-Rep68, DA-E2A, DA-E4ORF6, DA-VP1, DA-VP2, DA-VP3, DA-AAP, DA-MAAP, and/or DA-L4 100K.

64. The method of claim 62 or claim 63, wherein one or more of DA-Rep52, DA-Rep40, DA-Rep78, DA-Rep68, DA-E2A, DA-E4ORF6, DA-VP1, DA-VP2, DA-VP3, DA-AAP, DA-MAAP, and DA-L4 100K further comprises a non-synonymous codon, and wherein the method further comprises contacting the engineered cell with one or more single guide RNA(s) capable of targeting the Base Editor to the non-synonymous codon of DA-Rep52, DA-Rep40, DA-Rep78, DA-Rep68, DA-E2A, DA-E4ORF6, DA-VP1, DA-VP2, DA-VP3, DA-AAP, DA-MAAP, and/or DA-L4 100K.


SEQUENCE LISTING (45 sequences)

SEQ ID NO: 1 - DNA, 346 bp, synthetic construct
  source 1..346: mol_type = other DNA
ctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaattcgagctcggtacccgggga

SEQ ID NO: 2 - DNA, 423 bp, synthetic construct
  source 1..423: mol_type = other DNA
gtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctatcgatcggccggataacgccacc

SEQ ID NO: 3 - DNA, 496 bp, synthetic construct
  source 1..496: mol_type = other DNA
gaattctccaggcgatctgacggttcactaaacgagctctgcttatataggcctcccaccgtacacgccacctcgacatactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctccgcggggatcc

SEQ ID NO: 4 - DNA, 578 bp, synthetic construct
  source 1..578: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatg

SEQ ID NO: 5 - DNA, 571 bp, synthetic construct
  source 1..571: mol_type = other DNA
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatagttatc

SEQ ID NO: 6 - protein, 397 aa, synthetic construct
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 7 - protein, 312 aa, synthetic construct
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 8 - protein, 621 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 9 - protein, 536 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 10 - protein, 529 aa, synthetic construct
MASREEEQRETTPERGRGAARRPPTMEDVSSPSPSPPPPRAPPKKRLRRRLESEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 11 - protein, 294 aa, synthetic construct
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 12 - DNA, 888 bp, synthetic construct
  source 1..888: mol_type = other DNA
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaatgtttcctacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagtaa

SEQ ID NO: 13 - DNA, 738 bp, synthetic construct
  source 1..738: mol_type = other DNA
cgacgtaatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatcc

SEQ ID NO: 14 - protein, 735 aa, synthetic construct [sequence omitted]

SEQ ID NO: 15 - protein, 598 aa, synthetic construct
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 16 - protein, 533 aa, synthetic construct
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 17 - protein, 204 aa, synthetic construct
LETQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

SEQ ID NO: 18 - protein, 19 aa, synthetic construct
ATNFSLLKQAGDVEENPGP

SEQ ID NO: 19 - protein, 18 aa, synthetic construct
EGRGSLLTCGDVEENPGP

SEQ ID NO: 20 - DNA, 1932 bp, synthetic construct
  source 1..1932: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 21 - DNA, 1866 bp, synthetic construct
  source 1..1866: mol_type = other DNA
ctggcgggcttctacgagatcgtgatcaaggtgcccagcgacctggacgagcatctgcctggcatcagcgacagcttcgtgaattgggtcgccgagaaagagtgggagctgcctcctgacagcgacttggacctgaacctgattgagcaggcccctctgacagtggccgagaagctgcagagggatttcctgacagagtggcggagagtgtctaaggcccctgaggctctgttcttcgtgcagttcgagaagggcgagagctacttccacttacacgtgctggtcgagacaaccggcgtgaagtctttagtgctgggcagattcctgagccagatcagagagaagctgatccagcggatctaccggggcatcgagcccacactgcctaattggttcgccgtgaccaagaccagaaacggcgctggcggcggaaacaaggtggtggacgagtgctacatccccaactacctgctgcctaagacacagcccgaactgcagtgggcctggaccaacttagaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacatggaactcgttggctggctggtggacaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 22 - DNA, 3648 bp, synthetic construct
  source 1..3648: mol_type = other DNA
gccgccaccatggaattagtgggctggttggtcgataaaggcatcacaagcgaaaaacaatggattcaagaagatcaagcgagctatattagttttaacgccgctagtaatagcagaagtcagattaaagccgctctcgataacgccggcaaaatcatgtctttaaccaagacagctcctgattatttagtcgggcaacaacctgtcgaggacatcagttctaacagaatctacaagatcctcgaattgaatggctatgaccctcagtacgccgccagtgtgttcttaggctgggctaccaagaaatttgggaaacgcaatacaatttggttattcggccccgccaccacaggcaaaacaaatattgccgaagctatcgctcataccgtccctttctatggctgtgtgaattggacaaacgaaaatttcccttttaatgattgcgtggataaaatggtcatttggtgggaagaaggcaaaatgacagctaaagtggtcgaaagcgctaaggctatcttgggcggctctaaagtcagagtcgatcaaaagtgtaaaagtagcgctcaaatcgatcccacccctgtcattgtgacaagtaatacaaatatgtgtgctgtcatcgatggcaatagcaccacatttgagcatcaacaacccctccaggatagaatgtttaagttcgagttgacaagaagattagaccacgatttcggcaaagtgacaaaacaagaggtgaaggatttctttagatgggccaaagaccatgtcgtggaagtcgaacacgagttttatgtgaagaaaggcggcgctaaaaagcggcctgctccttccgatgccgacatctccgaacctaagagagtcagagaaagcgtggcccaacccagcaccagcgatgccgaggccagcattaattatgccgatcgctatcagaataagtgcagcagacatgtcgggatgaacttaatgttattcccttgtcggcagtgtgaacggatgaaccaaaacagcaacatttgttttacccacggacaaaaggattgcctggaatgtttccctgtcagcgagagccagcctgtgagcgtggtgaagaaagcctaccaaaagttatgttatatccaccacattatgggcaaagtccccgatgcctgtaccgcttgtgacttagtgaacgtagacctcgacgattgtattttcgagcagtgataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgcctggcttctacgagatcgtgatcaaggtgcccagcgacctggacgagcatctgcctggcatcagcgacagcttcgtgaattgggtcgccgagaaagagtgggagctgcctcctgacagcgacatggacctgaacctgattgagcaggcccctctgacagtggccgagaagctgcagagggatttcctgacagagtggcggagagtgtctaaggcccctgaggctctgttcttcgtgcagttcgagaagggcgagagctacttccacatgcacgtgctggtcgagacaaccggcgtgaagtctatggtgctgggcagattcctgagccagatcagagagaagctgatccagcggatctaccggggcatcgagcccacactgcctaattggttcgccgtgaccaagaccagaaacggtgctggcggcggaaacaaggtggtggacgagtgctacatccccaactacctgctgcctaagacacagcccgaactgcagtgggcctggaccaacatggaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacggagaactcgttggctggctggtggacaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 23 - protein, 1775 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSPKKKRKV

SEQ ID NO: 24 - protein, 1588 aa, synthetic construct
MSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGWRNAKTGAAGSLMDVLHYPGMNHRVEITEGILADECAALLCYFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 25 - protein, 1495 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQLPPLERLTLGSGGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 26 - protein, 1495 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQLPPLERLTLGSGGGGSQLHLPQVLADAVSRLVLGKFGDLTDNFSSPHARRKVLAGVVMTTGTDVKDAKVISVSTGGKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 27 - DNA, 12 bp, synthetic construct
  source 1..12: mol_type = other DNA
attggatccaat

SEQ ID NO: 28 - DNA, 28 bp, synthetic construct
  source 1..28: mol_type = other DNA
tatttacaaacaaccatgaatgtaagta

SEQ ID NO: 29 - DNA, 18 bp, synthetic construct
  source 1..18: mol_type = other DNA
cggagtactgtcctccga

SEQ ID NO: 30 - DNA, 30 bp, synthetic construct
  source 1..30: mol_type = other DNA
atgatacgaaacgtaccgtatcgttaaggt

SEQ ID NO: 31 - DNA, 26 bp, synthetic construct
  source 1..26: mol_type = other DNA
agaaacaaaccaacctgtctgtatta

SEQ ID NO: 32 - DNA, 28 bp, synthetic construct
  source 1..28: mol_type = other DNA
aacaaacagacaatctggtctgtttgta

SEQ ID NO: 33 - protein, 248 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 34 - protein, 298 aa, synthetic construct
MDMPRIKPGQRVMMALRKMIASGEIKSGERIAEIPTAAALGVSRMPVRIALRSLEQEGLVVRLGARGYAARGVSSDQIRDAIEVRGVLEGFAARRLAERGMTAETHARFVVLIAEGEALFAAGRLNGEDLDRYAAYNQAFHDTLVSAAGNGAVESALARNGFEPFAAAGALALDLMDLSAEYEHLLAAHRQHQAVLDAVSCGDAEGAERIMRDHALAAIRNAKVFEAAASAGAPLGAAWSIRADSGGGGPTDALDDFDLDMLPADALDDFDLDMLPADALDDFDLDMLPGPPKKKRKV

SEQ ID NO: 35 - protein, 264 aa, synthetic construct
MVRRTKEEAQETRAQIIEAAERAFYKRGVARTTLADIAELAGVTRGAIYWHFNNKAELVQALLDSLHETHDHLARASESEDEVDPLGCMRKLLLQVFNELVLDARTRRINEILHHKCEFTDDMCEIRQQHQSAVLDCHKGITLTLANVVRRGQLPGELDAERAAVAMFAYVDGLIRRWLLLPDSVDLLGDVEKWVDTGLDMLRLSPALRKSGGGGPTDALDDFDLDMLPADALDDFDLDMLPADALDDFDLDMLPGPPKKKRKV

SEQ ID NO: 36 - protein, 254 aa, synthetic construct
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCSGGGGPTDALDDFDLDMLPADALDDFDLDMLPADALDDFDLDMLPGPPKKKRKV

SEQ ID NO: 37 - protein, 257 aa, synthetic construct
MSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSIGLDLIVAADRDPALREGIQRTVERNRFVVEDMWLGVLVSRGLSRDDAEDILWLIFNSVRGLVVRSLWQKDKERFERVRNSTLEIARERYAKFKRSGGGGPTDALDDFDLDMLPADALDDFDLDMLPADALDDFDLDMLPGPPKKKRKV

SEQ ID NO: 38 - protein, 621 aa, synthetic construct; identical to SEQ ID NO: 8

SEQ ID NO: 39 - protein, 805 aa, synthetic construct
MESVEKEDSLTAPFEFATTASTDAANAPTTFPVEAPPLEEEEVIIEQDPGFVSEDDEDRSVPTEDKKQDQDDAEANEEQVGRGDQRHGDYLDVGDDVLLKHLQRQCAIICDALQERSDVPLAIADVSLAYERHLFSPRVPPKRQENGTCEPNPRLNFYPVFAVPEVLATYHIFFQNCKIPLSCRANRSRADKQLALRQGAVIPDIASLDEVPKIFEGLGRDEKRAANALQQENSENESHCGVLVELEGDNARLAVLKRSIEVTHFAYPALNLPPKVMSTVMSELIVRRARPLERDANLQEQTEEGLPAVGDEQLARWLETREPADLEERRKLMMAAVLVTVELECMQRFFADPEMQRKLEETLHYTFRQGYVRQACKISNVELCNLVSYLGILHENRLGQNVLHSTLKGEARRDYVRDCVYLFLCYTWQTAMGVWQQCLEERNLKELQKLLKQNLKDLWTAFNERSVAAHLADIIFPERLLKTLQQGLPDFTSQSMLQNFRNFILERSGILPATCCALPSDFVPIKYRECPPPLWGHCYLLQLANYLAYHSDIMEDVSGDGLLECHCRCNLCTPHRSLVCNSQLLSESQIIGTFELQGPSPDEKSAAPGLKLTPGLWTSAYLRKFVPEDYHAHEIRFYEDQSRPPNAELTACVITQGHILGQLQAINKARQEFLLRKGRGVYLDPQSGEELNPIPPPPQPYQQPRALASQDGTQKEAAAAAAATHGRGGILGQSGRGGFGRGGGDDGRLGQPRRSFRGRRGVRRNTVTLGRIPLAGAPEIGNRSQHRYNLRSSGAAGTACSPTQP

SEQ ID NO: 40 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 41 - protein, 735 aa, synthetic construct [sequence omitted]

SEQ ID NO: 42 - protein, 260 aa, synthetic construct
MVIMSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSIGLDLIVAADRDPVLREGIQRTVERNRFVVGDIWLGVLVSRGLSRDDAEDILWLIFNSVRGLVVRSLWQKDKERFERVRNSTLEIARERYAKFKRSGGGGPTDALDDFDLDMLPADALDDFDLDMLPADALDDFDLDMLPGPPKKKRKV

SEQ ID NO: 43 - protein, 119 aa, synthetic construct
LAHHHQSPQSGIRTTAGVLCFLGTSTSDPSTDSTRESRSTRQTPRPSSTTKPTTGSSTAETTRTSSTTTPTRSFRSALKKIRLLGATSDEQSSRRKRGFLNLWAWLRNLLRRLREKRGR

SEQ ID NO: 44 - protein, 1853 aa, synthetic construct
MKRTADGSEFESPKKKRKVSSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 45 - protein, 1999 aa, synthetic construct
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSMAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV